(19) United States
(12) Patent Application Publication
(10) Pub. No.: US 2024/0382573 A1
(43) Pub. Date: Nov. 21, 2024

(54) COMPOSITIONS INCORPORATING A GENETICALLY ATTENUATED PLASMODIUM WITH MODIFIED LIVER STAGE NUCLEAR PROTEIN (LINUP) AND RELATED METHODS

(71) Applicant: SEATTLE CHILDREN'S HOSPITAL D/B/A SEATTLE CHILDREN'S RESEARCH INSTITUTE, Seattle, WA (US)

(72) Inventors: Debashree GOSWAMI, Redmond, WA (US); Stefan H.I. KAPPE, Seattle, WA (US); Ashley Michael VAUGHAN, Seattle, WA (US)

(21) Appl. No.: 18/684,609

(22) PCT Filed: Aug. 19, 2022
(86) PCT No.: PCT/US2022/075238

§ 371 Date: Feb. 16, 2024

Related U.S. Application Data
(60) Provisional application No. 63/234,872, filed on Aug. 19, 2021.

(51) Int. Cl. A61K 39/015; A61K 39/00; A61P 37/04; C12N 1/10; C12R 1/90

(52) U.S. Cl. CPC A61K 39/015; A61P 37/04; C12N 1/10; A61K 2039/522; C12R 2001/90

(57) ABSTRACT

The present disclosure is directed to parasites that cause malaria, particularly Plasmodium-species parasites, and more particularly to Plasmodium-species parasites that have been genetically altered to develop normally only to the late liver stage but are completely inhibited in transition to blood stage or infection of erythrocytes. In particular, the inventors have identified genetic alterations that cause these parasites to arrest at the late liver stage of development, resulting from the disruption of the gene function of the liver stage nuclear protein (LINUP) gene. This complete late liver stage arrest allows for expression of an enhanced array of parasitic antigens throughout liver stage development, but prevents entry to blood stage, erythrocytic infection and the associated signs, symptoms and pathology of malaria disease.

### CROSS-REFERENCE(S) TO RELATED APPLICATION(S)

This application claims the benefit of U.S. Provisional Application No. 63/234,872, filed Aug. 19, 2021.

### STATEMENT OF GOVERNMENT LICENSE RIGHTS

This invention was made with Government support under R01 AI125706 awarded by National Institutes of Health. The Government has certain rights in the invention.

### STATEMENT REGARDING SEQUENCE LISTING

The Sequence Listing XML associated with this application is provided in XML format and is hereby incorporated by reference into the specification. The name of the XML file containing the sequence listing is 3399-P33WO.xml. The XML file is 300 KB; was created on Aug. 19, 2022; and is being submitted via Patent Center with the filing of the specification.

### BACKGROUND

This invention relates generally to malaria and the identification, creation and uses of genetically altered Plasmodium parasites. More particularly, the invention relates to genetically attenuated Plasmodium parasites, disrupted at single and/or multiple genetic loci, that can nevertheless proceed to late liver stage of development, but become attenuated at this stage and cannot proceed to blood stage.

Malaria has a tremendous impact on human health, killing hundreds of thousands annually and creating a major impediment for social and economic development of nations in malaria-endemic areas, particularly in sub-Saharan Africa [1]. Parasites of the genus Plasmodium, the causative agents of malaria, are transmitted to the vertebrate host via an infectious bite, in the saliva of an infected Anopheles mosquito. After transmission, Plasmodium parasites, specifically, the sporozoite stage of the life cycle, exit the bite site, enter the bloodstream of the host and migrate quickly through the blood stream to the liver. Sporozoites that infect hepatocytes and then grow and replicate within the infected hepatocyte as live stage parasites and mature from early liver stage through late liver stage, producing tens of thousands of blood stage-infectious merozoites, which are released into the blood stream and infect red blood cells. Here, they undergo further development and replication, after which they cause the rupture of the red blood cells, releasing a new wave of merozoites into the blood. Most of the new merozoites continue to repeat the replicative cycle by invading additional red blood cells. This cycling of infecting and rupturing of the red blood cells manifests in the potentially severe symptoms associated with malaria, such as fever, chills, weakness, malaise, an enlarged spleen, and death. A minority of the cycling merozoites, after invading red blood cells, develop into male or female gametocytes that remain in circulation within the body until being taken up in a blood meal through the bite of another mosquito. Assuming this mosquito is compatible for Plasmodium transmission (i.e., another Anopheles mosquito), the gametocytes proceed to develop into gametes and fuse to form a diploid zygote. Zygotes develop into motile ookinete forms, which penetrate the wall of the mosquito's midgut and form oocysts. Each oocyst undergoes numerous rounds of division to eventually produce infectious sporozoites, which accumulate in the mosquito's salivary glands and can then be injected into the next host, thus continuing the lifecycle.

The hepatic stage of Plasmodium infection is an attractive target for malaria prophylactic intervention as it is asymptomatic for this disease and precedes the symptomatic blood stage infection. Indeed, vaccines that target the liver stage of the life cycle have shown great promise in numerous clinical trials. To achieve this, vaccines have been attenuated at the liver stage of development by three distinct processes outlined below.

RADIATION-ATTENUTED VACCINE: Decades ago, it was found that irradiated Plasmodium-species sporozoites (“radiation attenuated sporozoites” or RAS) can confer sterile, protective immunity in host-specific rodents and humans when used as an experimental vaccine [2,3]. This was surprising, as a natural infection with malaria does not induce sterile protective immunity in endemic areas of the world. This approach has been used to produce radiation-attenuated P. falciparum (Pf) whole parasite vaccines that have proven to be highly efficacious in numerous clinical trials. Sanaria® PfSPZ Vaccine has been assessed to date in 19 clinical trials in 9 countries, is exceptionally safe and well tolerated, with rates of adverse events no different than in normal saline controls [4-12]. PfSPZ Vaccine has induced >90% vaccine efficacy (VE) against homologous challenge by controlled human malaria infection (CHMI) (same strain of Pf in vaccine and challenge) at 3-11 weeks after immunization: 80% VE against heterologous (different strains of Pf in vaccine and challenge) at 9.5 weeks (unpublished) after immunization; 54% heterologous VE against heterologous at 8 months [5, 7, 8]; and sterile VE durable for at least 18 months against intense naturally transmitted Pf malaria in 4 field trials in Africa, where VE against intensely transmitted Pf infection was 52%, 51%, and 47% for 6 months, and 58% in the second year by time to event analysis [9, 13]. PfSPZ attenuated by irradiation induces DNA damage and prevents DNA replication during liver stage development [2, 14, 15]. PfSPZ develop into approximately day 2 to 3 early liver stage parasites, but do not replicate. Plasmodium-specific immunogens produced during these liver stages elicit immunity primarily due to CD8+T cells, however, relatively large doses (9×10⁵ to 1.8×10⁶ PfSPZ) are required for engendering sterilizing immunity [4-9, 16, 17].

CHEMO-ATTENUATED VACCINE: Live, non-attenuated whole parasite-based vaccines administered under antimalarial drug cover (chemo-attenuation) targeting (i) the liver stage of the Plasmodium life cycle, or (ii) the first wave of parasite-infected red blood cells, have shown superior efficacy in clinical trials. In a recent trial [18], 3 doses of 2×10⁵ PfSPZ of Sanaria® PfSPZ-CVac (CQ) gave 100% protection against a heterologous CHMI at 12 weeks after last dose of vaccine. However, if chemo-attenuation relies on the blood stage anti-malarial, chloroquine (CQ), as does Sanaria® PfSPZ CVac Vaccine-CQ, there can be can a brief period of transient parasitemia 7-8 days after the initial injection of PfSPZ non-attenuated whole parasites, resulting in up to Grade 3 malaria symptoms unless ibuprofen is taken. In addition, if (for whatever reason) the partner drug is not taken, there is a risk of developing severe malaria.

GENETICALLY-ATTENUATED VACCINE: Genetically attenuated parasites (GAPs), are created by the deletion of specific genes that prevent complete liver stage development. GAPs have been extensively studied in rodent malaria models. The first GAPs were produced by deleting genes that are upregulated in infective sporozoites (UIS) as compared to oocyst sporozoites [19-22]. Such deletions did not affect viability of GAP when in the sporozoite stage, but resulted in growth arrest early in the liver stage of development before active liver stage replication takes place. Such GAPs are termed “early-arresting, replication deficient” (EARD) These GAPs exhibited favorable immunogenic properties, but have also, on occasion, exhibited incomplete attenuation, allowing for liver stage-to blood stage lifecycle progression (also called “breakthrough”), leading to an active infection. One early arresting GAPs, Sanaria® PfSPZ-GA1, was manufactured in compliance with GMP, and assessed in a clinical trial. It was shown to be well tolerated, safe, and completely attenuated as it did not show any breakthough blood stage infection, but conferred low efficacy against CHIM [23].

Next generation GAPs have been more recently developed in an attempt to create a parasite vaccine strain, which undergoes growth, development, and replication within the liver, only to arrest in development and die just before progressing to the blood stage [See, e.g. 24-27). These GAP are termed LARC (late-arresting, replication-competent) and these GAP are more powerful immunogens than the EARD GAP or RAS because they generate a significant vaccine biomass expansion in the liver and transit further through the liver life cycle, and thus present a larger and broader range of parasitic antigens to the immune system rendering the ensuing immune response more effective. Indeed, in the rodent malaria model, Plasmodium yoelii, LARC GAPs have been shown to provide superior protection from sporozoite challenge as compared to EARD GAPs and RAS. [28] Moreover, P. yoelii LARC GAPs have also been shown to provide stage-transcending protection from a direct blood stage challenge, indicating the presence of antigens in the late liver stage GAPs that are also characteristic of the blood stage forms [29]. However, published P. yoelii GAPs have either not successfully informed gene deletion in the human parasitic homologue P. falciparum and/or have shown in complete attenuation [26, 30]. Thus, there is a need to identify further targets for gene deletion to ensure complete attenuation. Ultimately, the aim is the creation of a human malaria LARC GAP that is completely attenuated in malaria clinical trials and can be used as a safe and highly efficacious vaccine to prevent malaria. However, the identification of additional target genes for deletion in human malaria-causing parasites, such as P. falciparum, which results in complete attenuation has been a continuing challenge.

Despite the advances in the art of creating attenuated Plasmodium parasites useful for stimulating the vertebrate immune system against a later infection, a need remains to identify novel specific genetic-based modifications, or novel combinations of multiple genetic-based modification, that provide complete attenuation, i.e., complete cessation of lifecycle progression in late liver stage development, without breakthrough to blood stage infection. This in turn will permit safe and prolonged development of the liver stage parasite vaccine immunogen to provide greater quantity and variety of antigens for a more complete immunity against subsequent parasitic infection. The present disclosure addresses these and related needs.

### SUMMARY

The present disclosure is directed to parasites that cause malaria, particularly Plasmodium-species parasites, and more particularly to Plasmodium-species parasites that have been genetically altered to develop normally to the late liver stage of development, but are blocked in the transition from liver stage to blood stage and subsequent infection of erythrocytes. Specifically, the inventors have identified genetic alternations that cause these parasites to be attenuated at the late liver stage of development by the deletion of the liver stage nuclear protein (LINUP) gene that result in the disruption of the LIMUP gene product, and this late liver-stage arrest. The arrested LINUP mutant allows for expression of an extensive parasite-specific antigenic array during liver stage development, but is severely attenuated for entry to blood stage, subsequent erythrocytic infection and the associated signs, symptoms and pathology of malaria disease. The family of genetically attenuated Plasmodium-species parasites comprising the LINUP disruption, including the double knockout, LARC2 (with deletion of both LINUP and PlasMei2 gene function), are ideal as live immunogens in malaria vaccines and related therapeutics.

### DESCRIPTION OF THE DRAWINGS

FIGS. 1A through IC. P. yoelii Liver stage Nuclear Protein (LINUP) (PY17X_1465200) localizes to the nucleus of liver stage parasites and will be referred to as Liver stage Nuclear Protein (LINUP).

A. Amino acid alignment between P. falciparum LINUP (PF3D7_1249700) (SEQ ID NO: 1) and its syntenic orthologs P. yoelii LINUP (PY17X_1465200) (SEQ ID NO:2) and P. vivax LINUP (PVP01_1466800) (SEQ ID NO:3) shows 40% amino acid sequence identity and 60% sequence similarity. See consensus sequence (SEQ ID NO: 4) Sequence identity/similarity is shown in gray. The predicted and conserved nuclear localization sequence (NLS) is underlined and spans amino acids 132-156.

B. To visualize the localization of P. yoelii LINUP, an mCherry epitope-tagged parasite strain, P. yoelii LINUP^{mCherry} was generated by fusing an mCherry tag to the LINUP C-terminus. The tagged transgenic parasite replaces the endogenous LINUP with the tagged copy. Immunofluorescence assays (IFAs) on P. yoelii LINUP^{mCherry} infected mouse livers using an mCherry antibody, an antibody to the endoplasmic reticulum marker BiP and the DNA strain DAPI, indicate that the protein is expressed after 24 hours of liver stage infection and at both 36 and 48 hours localizes to the nucleus of late liver stages (schizonts). Scale bar size is 5 μm.

C. At 48 hours, LINUP shows partial co-localization with the histone H3 marker acetylated lysine 9 and DAPI, scale bar is 5 μm, which is further indicated in the magnified image to the right where the scale bar size is 2 μm.

FIGS. 2A through 2E. P. yoelii LINNUP gene deletion parasite (linup) creation and analysis of mosquito stage development.

A. The schematic depicts the generation of the P. yoelii linup parasite using CRISPR/Cas9-mediated gene editing.

B. Blood stage growth was compared in groups of five Swiss Webster mice for P. yoelii XNL wildtype and P. yoelii linup clones c3 and c5. One million infected red blood cells were injected intravenously into each mouse and parasitemia measured every other day for ten days. Growth rates were comparable suggesting LINUP has no essential role in blood stage replication. Data is represented as mean+/−SEM, n=5 biological replicates. Statistical analysis was carried out using two-way ANOVA using Tukey's multiple comparison test. P>0.05 is taken as not significant. P. yoelii linup clones c3 and c5 did not have defects in mosquito infection as counts for:

C. oocysts/midgut:

D. oocyst prevalence; and E: salivary gland sporozoites/mosquito were comparable between P. yoelii linup c3 (dark gray) and c5 (light gray) to P. yoelii wildtype (black). Data is represented as mean+/−SD, n=3 biological replicates. Statistical analysis was carried out using two-way ANOVA using Tukey's multiple comparison test. P>0.05 is taken as not significant.

E. salivary gland sporozoites/mosquito were comparable between P. yoelii linup⁻ c3 (dark gray) and c5 (light gray) to P. yoelii wildtype (black).

Data is represented as mean+/−SD, n=3 biological replicates. Statistical analysis was carried out using two-way ANOVA using Tukey's multiple comparison test. P>0.05 is taken as not significant.

FIGS. 3A through 3D. Analysis of P. yoelii linup⁻ liver stage development. Tissue sections were prepared from BALB/cJ mice infected with 250,000 sporozoites of either P. yoelii wildtype or P. yoelii linup⁻ at 24, 36 and 48 hours post infection and analyzed by IFA.

(A). Comparison of the size of liver stage parasites (based on area at the parasite's largest circumference) between P. yoelii wildtype and P. yoelii linup⁻ at 24, 36 and 48 hours indicate that P. yoelii wildtype liver stage schizonts are significantly larger than P. yoelii linup⁻ at 36 and 48 hours. Data is represented as mean±SD. Each datapoint refers to the mean size of at least 20 parasites for each timepoint. Statistical analysis was carried out using two-way ANOVA using Tukey's multiple comparison test. *=P.05, ***P<0.001, P>0.05 is taken as ns. Liver stage development was compared at 48 hours using antibodies against:

(B). the parasite mitochondria (mHSP70) and apicoplast (ACP):

(C). the parasite plasma membrane and mature exo-erythrocytic merozoite marker, MSP1; and,

(D). the inner membrane complex protein and mature exo-erythrocytic merozoite marker, mTIP. DNA was stained with DAPI. Scale bar: 10 μm. P. yoelii linup⁻ liver stage parasites are smaller compared to P. yoelii wildtype and display less branching of the mitochondria and apicoplast organelles. P. yoelii linup⁻ expresses the late liver stage proteins MSP1 and mTIP, however there is aberrant segregation of cytomeres and incomplete formation of mature exo-erythrocytic merozoites.

FIGS. 4A through 4E. P. falciparum linup creation and analysis of mosquito stage development.

A. The schematic depicts the generation of P. falciparum linup knockout parasites using CRISPR/Cas9-mediated gene editing. Primers used to verify the gene deletion are indicated and the sizes of the PCR amplicons are shown in kilobases:

B. Agarose gel electrophoresis shows the PCR products corresponding to the gene deletion of P. falciparum LINUP in clones B1 and B4. P. falciparum linup did not have defects in mosquito infectivity as counts for:

C. oocysts/midgut:

D. oocyst prevalence; and,

E. salivary gland sporozoites/mosquito were comparable between P. falciparum linup⁻ clones B1 (dark gray) and B4 (light gray) and P. falciparum NF54 (black). Data is represented as mean+/−SD, n=2 biological replicates. Statistical analysis was carried out using two-way ANOVA using Tukey's multiple comparison test. P>0.05 is taken as not significant.

FIGS. 5A through 5B. P. falciparum linup⁻ liver stages displays abnormal growth and aberrant late liver stage development. Tissue sections were prepared from FRG NOD huHep mice that were infected with one million sporozoites of P. falciparum NF54 and P. falciparum linup⁻ on days 5 and 7 post-sporozoite infection and analyzed for size and protein expression.

A. Comparison of the size of liver stage parasites (based on area at the parasite's largest circumference) between P. falciparum NF54 and P. falciparum linup⁻ on days 5 and 7 post-sporozoite infection. While no growth defect was seen between P. falciparum NF54 and P. falciparum linup⁻ liver stage schizonts on day 5, there was a statistically significant difference in the size of late liver stage schizonts on day 7. Data is represented as mean±SD. Each datapoint refers to the mean size of at least 30-50 parasites for each timepoint. Statistical analysis was carried out using two-way ANOVA using Tukey's multiple comparison test. ***P<0.001, P>0.05 is taken as ns.

B. IFA shows that at seven days of infection the wildtype NF54 parasitophorous vacuole membrane protein EXPI has a circumferential staining pattern in the wildtype, as expected. In comparison EXPI expression in mature P. falciparum linup⁻ at day seven of the liver stage is patchy and aberrant.

FIGS. 6A through 6C. P. falciparum linup⁻ display severe attenuation of liver stage development and transition to blood stage in infected FRG NOD huHep humanized mice.

A. Schematic shows the experimental design of liver stage-to-blood stage transition experiments in FRG NOD huHep mice. One million sporozoites from P. falciparum NF54 and P. falciparum linup⁻ were injected into one and three FRG NOD huHep mice, respectively. Mice were repopulated with human red blood cells as depicted. All mice were euthanized on day seven and 50 μl blood samples were collected from all mice for parasite load based on 18S rRNA qRT-PCR sampling. The remaining blood was transferred to in vitro culture and grown in culture for seven days. Further 50 μl blood samples were collected from in vitro culture samples for analysis of parasite 18S rRNA qRT-PCR samples seven days after transition to in vitro culture (14 days after sporozoite infection).

B. Analysis of parasite load by 18S rRNA qRT-PCR was carried out on extracted RNA from the blood of mice infected with P. falciparum NF54 and P. falciparum linup⁻ P. falciparum linup⁻ sporozoites on day 7, after expected completion of liver stage development and liver stage-to-blood stage transition (Day 7, black bars). There was a severe defect in the P. falciparum linup⁻ transition to blood stage infection as one out of three mice infected with P. falciparum linup⁻ sporozoites did not shown blood stage in the blood, while the other two infected mice had a 10,000-fold reduction in parasite RNA load as compared to wildtype. Blood was transitioned to in vitro culture and parasite load was measured after a further seven days (day 14, gray bars). Wildtype NF54 blood stages replicated during the seven days in culture, whilst no significant replication was seen for P. falciparum linup⁻ blood stages. This shows that the liver stage-to-blood stage transition of P. falciparum linup⁻ did not release viable blood stage parasites that can initiate blood stage replication.

C. Analysis of parasite liver load by 18S rRNA qRT-PCR was carried out on extracted RNA from P. falciparum NF54 and P. falciparum linup⁻ infected livers on day 7 post infection, depicted as copies of P. falciparum 18S rRNA/μg of extracted liver RNA. Parasite liver burden is comparable between P. falciparum NF54 and P. falciparum linup⁻.

FIGS. 7A through 7D. Generation of P. falciparum plasmei2/linup (P. falciparum LARC2).

A. The cartoon depicts the generation of the P. falciparum plasmei2 parasite using CRISPR/Cas9-mediated gene editing. Recombinant parasites were cloned by limiting dilution. The cartoon depicts the generation of the P. falciparum linup parasite using CRISPR/Cas9-mediated gene editing. Marker free P. falciparum PlasMei2 clone F3 from (A) was used for transfection of the P. falciparum LINUP KO plasmid. Recombinant parasites were cloned by limiting dilution to generate P. falciparum plasmei2/linup double knockout (LARC2) clones. Primers used to verify the gene deletion are indicated. The combination of primers used and the sizes of the PCR products is indicated in Table 2. Agarose gel electrophoresis for lack of P. falciparum PlasMei2

B. lack of P. falciparum LINUP

C. and lack of plasmid DNA

D. in four P. falciparum LARC2 clones-C12, F7, B7, and B5.

FIGS. 8A and 8B. Lack of P. falciparum PlasMei2 and P. falciparum LINUP in P. falciparum LARC2 clones. Eight P. falciparum LARC2 clones were analyzed by whole genome sequencing to confirm:

A. deletion of P. falciparum PlasMei2 on chromosome 6; and.

B. deletion of P. falciparum LINUP on chromosome 12. P. falciparum LARC2 clones B7, C12, D5 and F7 will be used for further phenotypic analysis.

FIGS. 9A through 9D.

A. Exflagellation centers were counted in mature gametocyte cultures of P. falciparum LARC2 on day 15 post set up for four P. falciparum LARC2KO clones B7, C12, D5 and F7 and found to be comparable to P. falciparum NF54. These gametocyte cultures were fed to female Anopheles stephensi mosquitoes in standard membrane feeding assays and on day 8 post feed midguts were dissected:

B. Oocyst prevalence:

C. counts for oocyst/mosquito were comparable to wildtype for P. falciparum LARC2KO clones F7 and B7. These two clones will be used for further phenotypic analysis; and

D. counts for sporozoites/mosquito were comparable to wildtype for P. falciparum LARC2 clones F7 and B7. Clone F7 was used for further phenotypic analysis.

FIGS. 10A through 10C. P. falciparum LARC2 parasites are severely attenuated in liver stage development and fail to generate infectious exo-erythrocytic merozoites.

A. The schematic depicts the experimental design. P. falciparum NF54 and P. falciparum LARC2 sporozoites were isolated from salivary glands of infected Anopheles stephensi mosquitoes. To evaluate for blood stage transition of P. falciparum LARC2 in FRG NOD huHep mice, 1×10⁶ aseptic cryopreserved P. falciparum NF54 (PFSPZ) and aseptic cryopreserved P. falciparum LARC2 (PFSPZ LARC2) sporozoites were injected intravenously into four and six FRG NOD huHep mice per group respectively. On day 6 and 7, 400 μl of 70% RBCs were injected intravenously to enable transition of liver stage parasites to blood. Four hours after human RBC repopulation on day 7, mice were euthanized, blood was collected by cardiac puncture and 50 μl of blood from each mouse was used for qRT-PCR analysis to detect parasite 18S RNA. Blood was washed three times in asexual media, a volume of human RBCs equal to the packed RBC volume was added and blood was transferred to in vitro culture. Fresh media was replaced daily, and cultures were analyzed every 2-3 days by thick smear for presence of parasites for up to 6 weeks.

B. On day 7, parasite densities for mice infected with PfSPZ ranged between 10³-10⁵ parasite equivalents/ml by P. falciparum 18S qRT-PCR, which further increased to 10⁹ parasite equivalents/mml after seven days of in vitro culture. In contrast, in mice infected with PFSPZ LARC2, 0-10³ parasite equivalents/ml were detected by P. falciparum 18S qRT-PCR and after seven days in culture there was a drastic decline in P. falciparum 18S rRNA signal, indicating that no viable exo-erythrocytic merozoites were released from PFSPZ LARC2 liver stage schizonts. This was further confirmed by thin blood smears. Blood stage parasites were detected by Giemsa-stained thin blood smears in all four mice infected with PfSPZ within 1-3 days of transition to in vitro culture (see Table 5). In contrast, no blood stage parasites were detected by Giemsa-stained thin blood smears in all six mice infected with PfSPZ LARC2.

(C) Livers of infected mice were also harvested on day 7 for extracting RNA and analysis of parasite liver burden by P. falciparum 18S qRT-PCR. Parasite liver load is depicted as log 10 copies of P. falciparum 18S rRNA per μg of total liver RNA. Parasite liver burden was comparable between PfSPZ and PfSPZ LARC2, indicating that LARC2 persists in the liver until day 7 and undergoes significant biomass expansion.

### DETAILED DESCRIPTION

### Definitions

“Additive” as used herein as a noun is a compound or composition added to a sporozoite preparation. Additives include diluents, carriers, excipients cryoprotectants and the like.

“Aseptic” as used herein means absent the introduction of detectable contamination of other microorganisms such as bacteria, fungi, pathologic viruses and the like. Aseptic sporozoite preparations results in a sterile preparation of sporozoites-free of any other type of microorganism or infectious agent. Microbiological assays used to monitor an aseptic methodology assess the presence or absence of contamination. They include, but are not limited to, the Microbial Limits Test, current USP <61>, incorporated herein by reference.

“Attenuate” as used herein means to render a live organism unable to complete its life cycle without killing it. The organism may have a limited capacity to replicate, express proteins, and to develop through some life cycle stages, but arrests development at a particular life cycle stage and is unable to developmentally progress beyond that stage. With regard to the attenuated Plasmodium parasites disclosed herein, they retain the ability to infect host hepatocytes and express stage specific proteins, but are unable to develop beyond liver stage, are unable to transition to the blood stage infections in the blood stream of infected hosts after liver-stage development and are unable to cause the disease pathology of malaria.

“Challenge” as used herein refers to the presentation of an infectious pathogen to a subject that has been previously been provided with a vaccine intended to confer a protective immunity against infection/disease caused by the challenging pathogen. With regard to malaria, the challenge can be by (CHMI, [31, 32] inoculation of infectious Plasmodium sporozoites: by the exposure to Anopheles mosquitoes carrying infectious Plasmodium sporozoites: or field trials in which vaccinated subjects are monitored in a region where malaria parasites are naturally transmitted by infected mosquito bite.

“Conferring protective immunity” as used herein refers to providing to a population or a host (i.e., an individual) the ability to generate an immune response to protect against an infection/disease (e.g., malaria) caused by a pathogen (e.g., Plasmodium) such that the clinical manifestations, pathology, or symptoms of disease in a host are reduced as compared to a non-treated host, or such that the rate at which infection, or clinical manifestations, pathology, or symptoms of disease appear within a population are reduced, as compared to a non-treated population.

As used herein, the term “disrupt” with regard to gene function, means interfering with the gene function such as to inhibit, inactivate, attenuate, or block the gene function or the function of the encoded gene product. The interference or disruption can be accomplished, for example, by altering the gene sequence (e.g., substitution, modification, deletion, addition, knockdown, knock out or knock in) in a manner and/or to degree such that the translated protein, if any, no longer performs its wildtype function. Alternatively, the gene sequence can be deleted.

As used herein, the term “genetically modified” refers to a modification to the genome of the wildtype Plasmodium organism that results in a defined difference from the wildtype genome sequence. The genetic modification is imposed by human manipulation, e.g., by genetic engineering. With regard to the invention disclosed herein, the genetic modification may be one or more insertions or deletions in a gene, knockout of the gene, or other modifications. In these inventions, genetic modification results in functional disruption of the LINUP gene and in certain embodiments, the LINUP gene and the PlasMei2 gene.

“Immune response” as used herein means a response in the recipient to the introduction of attenuated sporozoites generally characterized by, but not limited to, production of antibodies and/or T cells. Generally, an immune response may be a cellular response such as induction or activation of CD4+ T cells or CD8+ T cells specific for Plasmodium species epitopes, a humoral response of increased production of Plasmodium-specific antibodies, or both cellular and humoral responses. With regard to a malaria vaccine, the immune response established by a vaccine comprising live sporozoites includes but is not limited to responses to proteins expressed by extracellular sporozoites, the intracellular liver stages or other stages of the parasite. Mononuclear cells such as dendritic cells will take components of said parasites and present these antigens to relevant immune cells. In the instant invention, upon subsequent challenge by infectious organisms, the immune response prevents development of pathogenic parasites to the asexual erythrocytic stage that causes disease.

As used herein, the term “live” refers to continued metabolic activity in the Plasmodium organism. In some embodiments “live” indicates that the Plasmodium organism is capable of eventually establishing at least a transient infection, for example within hepatocytes (cultured or in vivo). The Plasmodium organism can be in any relevant developmental stage as is practical considering the genetic attenuation. Thus, for example, the Plasmodium organism can be in the intra-mosquito developmental stages, infective sporozoite stage, or intra-hepatocytic (liver) stage.

Sporozoites produced “in vitro”, or “iSPZ” as used herein means sporozoites that have developed external to mosquitoes, i.e, wherein sporogony from gametocyte stage to mature, infectious sporozoite stage is external to mosquitoes [33].

“Metabolically active” as used herein means alive, and capable of performing sustentative functions and some life-cycle processes. With regard to attenuated sporozoites this includes but is not limited to sporozoites capable of invading hepatocytes in culture and in vivo, potentially having a substantial capacity to divide and progress through some developmental stages, and de novo expressing stage-specific proteins.

As used herein, the terms “Plasmodium organism” or “Plasmodium-species” refer to any parasite that belongs to the genus Plasmodium. In some embodiments, the Plasmodium organism has a human host range, for example, P. falciparum, P. vivax, P. ovale, P. malariae, and P. knowlesi. In some embodiments, the Plasmodium organism is P. falciparum.

“Promoter” as used herein is a region of DNA upstream of a gene where relevant proteins (such as RNA polymerase and transcription factors) bind, to initiate and regulate transcription of that gene.

“Transgene” as used herein is a gene that is not normally occurring in the organism to which the transgene has been introduced. In some embodiments of the current invention, the transgene, or expression of the encoded polypeptide, serves to disrupt one or more gene function of the organism. In other embodiments, the transgene encodes a foreign antigen, the expression of which elicits an immune response.

“Vaccine” as used herein is a preparation comprising an immunogenic agent and a pharmaceutically acceptable additive, e.g. excipient, adjuvant and/or additive or protectant. The immunogen may be comprised of a whole infectious agent or a molecular subset of the infectious agent (produced by the infectious agent, synthetically or recombinantly). When the vaccine is administered to a subject, the immunogen stimulates an immune response that will, upon subsequent challenge with infectious agent, protect the subject from illness or mitigate the pathology, symptoms or clinical manifestations caused by that agent. A therapeutic (treatment) vaccine is given after infection and is intended to reduce or arrest disease progression. A preventive (prophylactic) vaccine is intended to prevent initial infection or reduce the rate or burden of the infection.

The present disclosure is directed to genetically attenuated malaria parasites that develop to the late liver stage, but are blocked in the transition to blood stage of infection of erythrocytes. In particular, the attenuated malaria parasites comprise genetic alternations that disrupt the functionality of the liver stage nuclear protein (LINUP) gene. In some embodiments, the malaria parasites comprise Plasmodium-species of human host range, e.g. P. falciparum. P. vivax. P. malariae. P. ovale. P. knowlesi. In some embodiments the malaria parasites contain additional genetic modifications wherein other genes critical for the transition from liver to blood stage are interrupted. As a non-limiting example, the malaria parasite of an embodiment additionally comprises genetic modifications wherein both the LINUP and the PlasMei2 gene functionalities are disrupted (this double knockout is variously referred to as “linup/plasmei2 double knockout (KO)”, “LARC2”, “LARC2 KO” and these terms are equivalent). The disclosed attenuated malaria parasites are useful for compositions comprising additives, vaccines for the prevention and amelioration of malaria, methods for inducing and/or stimulating human immune systems against Plasmodium-specific antigens, methods for inducing an immune response in a human subject against one or more Plasmodium antigens, or methods for conferring protective immunity in a human subject against malaria caused by a Plasmodium-species parasite, which are also encompassed by the present disclosure. In some embodiments, the disclosed attenuated malaria parasites are useful for compositions and methods for ameliorating, reducing, preventing, treating, and/or protecting against malaria infection.

As described in more detail below, the inventors discovered a novel protein (LINUP), described as a liver stage nuclear protein, or as a liver-stage-specific protein that localizes to the parasite nucleus. Knockout of the gene or gene function encoding this protein results in life-cycle arrest late in liver stage development. Additionally, the inventors developed a double knockout, referred to as LARC2 (late arresting replication competent double KO), which contains a functional deletion of both the PlasMei2 and LINUP genes. This double knockout is a superior immunogen compared to the previous generations of replication deficient whole parasite based vaccines, because it undergoes near complete development in the liver, to late liver stage, resulting in expansion of parasite antigen biomass and diversity. In addition, LARC2 is more securely attenuated due to genetic manipulation of two genes required for transition to blood stage, and also ensures batch-to-batch homogeneity, and overcomes the need for antimalarial drug cover, which is a major limitation following immunizations with live non-attenuated sporozoite vaccines under drug cover, i.e., chemo-attenuated vaccination (another late liver stage approach described above). Genetically attenuated Plasmodium-species (e.g., P. falciparum) with a genetic knockout of LINUP, and in another embodiment, another liver stage specific gene such as PlasMei2, or in still other embodiments, other genes encoding proteins necessary for transit from liver stage to blood stage, can be used to induce an immune response in a subject, particularly a human subject, against one or more Plasmodium-species antigens, for conferring protective immunity in a subject, particularly a human subject, against malaria caused by a Plasmodium-species parasite, for immunization of a subject, particularly a human subject, as a pre-erythrocytic stage vaccine that provides or boosts immunity against re-infection and/or to treat, prevent, or ameliorate malaria infection. The complete interruption of the Plasmodium-species parasite life cycle at the liver stage also results in the prevention of parasite transmission to other individuals. This is vital in elimination campaigns to eradicate malaria.

In an embodiment, a live Plasmodium-species organism is genetically modified to disrupt a LINUP gene that encodes a Liver Stage Nuclear Protein, thereby preventing the biological function of a protein encoded by the wildtype LINUP gene.

In an embodiment, the live Plasmodium-species linup⁻ parasite arrests life cycle development in late liver stage within a mammalian intermediate host.

In other embodiments, the Plasmodium-species linup⁻ or LARC2 parasite comprises at least one transgene, and in still other embodiments, the transgene is under control of a promoter that results in transcription of the transgene during the sporozoite or liver stage of development. In certain embodiments, one or more transgene encodes a blood stage- or gametocyte-associated antigen. By providing for the expression of one or more blood stage- or gametocyte-associated antigens, the GAP will possess additional immunogenicity and provide protection against blood stage parasites (asexual and sexual). Thus, even if the GAP fully arrests prior to development into a blood stage, it will still be able to stimulate the immune cells against antigens characteristic of blood stage parasites. This provides further protection against blood stage parasites and reduces the risk of clinical symptoms as well as transmission of infection in the gametocyte antigens. In other embodiment, one or more transgenes encode antigens from other pathogens, and thus provide an immune response to these additional pathogens.

In an embodiment, the Plasmodium-species organism is P. falciparum. P. vivax. P. malariae. P. ovale, or P. knowlesi.

In an embodiment, the Plasmodium-species organism is at the sporozoite stage of development.

In an embodiment, the functional LINUP gene of the live Plasmodium-species linup⁻, prior to genetic modification has at least about 40%, 50%, 60% 70%, 80%, 90%, or 95% nucleotide sequence identity to SEQ ID NO: 35.

In an embodiment, in the live Plasmodium-species linup⁻ parasite, the functional LINUP gene prior to genetic modification encodes a LINUP polypeptide with at least about 40%, 50%, 60% 70%, 80%, 90%, or 95% sequence identity to the amino acid sequence set forth in PF3D7_1249700, SEQ ID NO: 1.

In an embodiment a vaccine composition comprises Plasmodium-species linup⁻ parasite at the sporozoite stage, and an additive, where the vaccine composition is prepared aseptically.

In an embodiment, the Plasmodium-species linup⁻ parasite is genetically modified to disrupt one or more additional gene functions, each additional gene function necessary for the liver stage to blood stage transition, and in another embodiment, the additional gene function is PlasMei2. In certain embodiments the functional PlasMei2 gene prior to genetic modification comprises a nucleic acid sequence that is at least at least about 70%, 80%, 90%, or 95% identity to the nucleic acid sequence set forth in SEQ ID NO:34. In certain embodiments, the amino acid sequence of a functional PlasMei2 polypeptide encoded by the PlasMei2 gene comprises is at least at least about 70%, 80%, 90%, or 95% identity to the sequence set forth in SEQ ID NO: 36.

In an embodiment a vaccine composition comprises the Plasmodium-species LARC2 (linup−/plasmei2−) and an additive. In certain embodiments, this vaccine composition is prepared aseptically.

Other embodiments relate to methods for inducing an immune response in a human subject against one or more Plasmodium antigens, comprising administration of one or more doses of the Plasmodium-species linup⁻ vaccine composition to the subject, and still other embodiments relate to methods for inducing an immune response in a human subject against one or more Plasmodium antigens, comprising administration of one or more doses of the Plasmodium-species LARC2″ vaccine composition to the subject. Still other embodiments relate to methods wherein the immune responses generated by the administration of these vaccines ameliorates or protects against infection from a subsequent wildtype Plasmodium challenge. Still other embodiments relate to methods of conferring protective immunity in a human subject against malaria caused by a Plasmodium-species parasite by administration of these vaccines.

In an embodiment, Plasmodium-species LINUP″ sporozoites are produced in vitro, and in an embodiment, Plasmodium-species LARC2″ sporozoites are produced in vitro. Plasmodium-species sporozoites produced in vitro have been disclosed [34], and particularly, genetically attenuated Plasmodium-species sporozoites produced in vitro have been disclosed [35], and both of these references are incorporated herein in their entirety.

### Purification Methods

Methods of purifying live organisms about 10 microns in size, such as Plasmodium-species sporozoites, e.g. P. falciparum SPZ, are known in the art as exemplified by [36], and particularly genetically attenuated Plasmodium-species SPZ [37]. These methods utilize a series of size exclusion filters of different types and with different pore sizes, assembled in a non-intuitive fashion. The methodology eliminates attendant material from preparations of live, motile parasites. A unique aspect of this method is that the pore size of a size exclusion filter in sequence is not always smaller than the pore size of the size exclusion filter which precedes it. Another inventive aspect is that some filters provide a matrix with a nominal pore size and at least one filter provides a track-etched filter with a precise pore diameter. At least one filter has a pore size close to or slightly smaller than the diameter of the parasite.

Typically in a preparation for purification, the salivary glands from 150 to 400 mosquitoes are dissected. The sporozoites are released from the salivary glands by passage back and forth in a needle and syringe (trituration), and sporozoites from these glands are collectively purified. However, several fold more mosquitoes may be dissected in scaled up preparations, in an embodiment up to 1,000 mosquitoes, in another embodiment up to 5,000 mosquitoes, in another embodiment up to 10,000 mosquitoes. Sporozoites are released from salivary glands by trituration and the triturated salivary gland preparations (pre-purification preparations) are purified by the size exclusion filtration process disclosed herein. Sporozoites are maintained throughout the purification process in an excipient, typically one percent human serum albumin (HSA) in Medium 199 with Earle's salts (E-199).

### A—Preparation of Material for Purification

The triturated dissection product (pre-purification preparation) is received altogether in a single tube at a time. This is the mosquito salivary gland material (SGM) pre-purification preparation. It represents about 100,000 to 1 billion sporozoites, preferably at least 1 million sporozoites and more preferably at least 25 million sporozoites. The measured amount of SGM in the pre-purification preparation is usually between 300 ng and 12,000 ng per 25,000 sporozoites, more typically, between 400 ng and 1,100 ng per 25,000 sporozoites. The pre-purification preparation is then diluted to 10 ml with excipient. Solutions and samples are kept between 15-30° C. for the duration of the purification.

### B—Purification Procedure

Using a peristaltic pump the diluted pre-purification preparation is pumped across a series of size exclusion filters at a flow rate of at least 1 ml/min but no more than 1000 ml/min, preferably at least 2 ml/min, but no more than 500 ml/min, and more preferably with a flow rate of at least 3 ml/min and no more than 200 ml/min. The corresponding flux across each filter is at least 1 L/hr/m² but no more than 2000 L/hr/m², preferably 3 L/hr/m² to 1500 L/hr/m², and most preferably at least 125 L/hr/m² but no more than 250 L/hr/m². Filters are connected in series, usually with medical grade silicone tubing. Preferably, the initial filter (Filter #1) or the initial two filters (Filters #1 and #2) are matrix filters and are made of polypropylene, however, nylon, mixed cellulose ester and borosilicate glass or other material known to those in the art may be used. Preferably, the penultimate filter (Filter #3) is a membrane filter, most preferably a track-etched polycarbonate filter, although other filters with similar properties known to those in the art may be used. For aseptic procedures, the filters are sterile. In an embodiment, three filters (Filter #1, Filter #2 and Filter #3) are connected in series and sporozoites are captured by dead end filtration on Filter #4. Additional filters may be used. In an embodiment, filter #1 is a membrane matrix with a nominal pore size of at least about 2.5 microns, but no more than about 30 microns, preferably at least about 5 microns, but no more than 20 microns. In one embodiment the filter used has a nominal pore size of about 10 microns with a filtration area of 17.5 cm². (Polygard®-CN Optiscale-Millipore Cat. No. SNIHA47HH3). In a scaled up embodiment the filtration area is 1800 cm². The nominal pore size of Filter #2 (also a membrane matrix) is at least about 0.3 microns but not larger than about 1.2 microns. In one embodiment, the nominal pore size is about 0.6 micron with a filtration area of 17.5 cm² (PolygardR-CN Optiscale filter-Millipore Cat. No. SN06A47HH3)—smaller than the diameter of the Plasmodium sporozoite. In a scaled up embodiment the filtration area is 1800 cm². In one embodiment, Filter #3 is a track-etched membrane filter with precise pore diameter and consistent pore size, and has a pore size of at least 1.2 microns but not larger than 3 microns-larger than the nominal pore size of the preceding filter. In one embodiment the filter used has a pore size of 1.2 microns with a filtration area of 11.3 cm². (Isopore membrane, 47 mm in diameter-Millipore Cat. No. RTTP04700) held in a Swin-Loc filter holder (Whatman Cat. No. 420400). In a scaled-up embodiment the filtration area is 127 cm². Filtered material is captured on Filter #4 in a stirred ultrafiltration cell (Millipore, model 8200) fitted with an Isopore membrane, 90 mm in diameter with a filtration area of 28.7 cm², and a track-etched pore size of no more than 0.8 microns, preferably no more than 0.6 microns, and preferably no more than 0.2 microns. In one embodiment the pore size is 0.4 microns (Millipore Cat. No. HTTP09030). In a scaled-up embodiment the filtration area is 162 cm². In another scaled-up embodiment the filtration area is 63 cm². The system is washed several times with media. When the retentate volume reaches about 40 ml in the stirred cell, the stirred cell container outlet is opened and drained by gravity leaving about 5-10 ml of residual retentate although the retentate volume can be reduced by other methods such as applying pressure from compressed gas such as nitrogen or a mechanical device such as a piston, gravity is the preferred method. This residual retentate is collected and transferred, together with three washes using purification media to a total of about 35 ml, typically in a sterile 35 ml Oak Ridge or similar centrifuge tube (the size of the tube will vary depending on the volume of the preparation). Purified sporozoites in media in the 35 ml Oak Ridge tube are centrifuged at 5,000 g to 25,000 g, preferably at 16,300 g, for 2 minutes to 12 minutes, preferably five minutes, to pellet the sporozoites. The supernatant media is decanted. This step additionally purifies the sporozoite preparation by removing smaller more buoyant materials and soluble materials that remain in the supernatant.

Using the 3 size-exclusion filter methodology, this procedure provides greater than a substantial reduction of attendant material in the purified sporozoite preparation relative to the attendant material in the pre-purification preparation (reduction factor) of from 200 to 10,000-fold. The amount of residual SGM in purified preparations of sterile purified sporozoites routinely is less than 25 ng of attendant material per 25,000 sporozoites (greater than 97% reduction relative to the initial amount of SGM), preferably less than 15 ng per 25,000 (98% reduction) sporozoites, and more preferably less than 1 ng per 25,000 sporozoites (99.9%). The contaminating SGM in each purified preparation described herein is usually reduced several thousand-fold relative to SGM in the initial triturated pre-purified salivary gland material from which each purified preparation is derived. Preferably the purification reduction factor is at least 15-fold, more preferably, the purification factor is at least 1,500-fold and most preferably at least 3,500-fold. The geometric mean of the reduction factor in the 10 campaigns described in Example 1 is 1625, a 99.93% reduction in SGM during the purification process.

This method is effective at reducing contamination and attendant mosquito salivary gland material in a crude sporozoite preparation. The sporozoites may then be cryopreserved as is also known in the art.

### Vaccine Compositions and Methods of Use

In some embodiments, Plasmodium-species sporozoites, and particularly purified Plasmodium-species sporozoites, are prepared aseptically. This methodology is known in the art as exemplified by and further exemplified in the description of the GMP manufacture of aseptic, purified Pf-LARC2 in Example 7, below.

Genetically attenuated Plasmodium-species sporozoites, including such vaccines in which the immunogen is Plasmodium-species P. falciparum linup⁻ and Plasmodium-species P. falciparum-LARC2, are generally administered in a regimen of one to three doses, and usually are administered parenterally, including subcutaneous, intradermal and intravenous administration, preferably by intravenous direct venous inoculation (DVI) methodology, known in the art. A suitable dose of genetically attenuated Plasmodium sporozoites, such as P. falciparum genetically attenuated sporozoites, per inoculation may be between about 10,000 to about 10 million sporozoites, preferably between 100,000 and 1,000,000 sporozoites. In some embodiments of the invention, the vaccine is administered with an adjuvant, for example 7DW8-5 as described in [39], and such administration in conjunction with adjuvant would likely reduce and number of sporozoites required per dose.

Such vaccines are useful for prevention or reduction of severity of malaria, its manifestations, symptoms or its pathology. In an embodiment, compositions and vaccines comprising aseptically prepared genetically attenuated purified sporozoites provide partial, enhanced, or full protection in human and other mammalian subjects not previously exposed to a malaria-causing pathogen, or exposed, but not fully protected. These compositions and vaccines are similarly useful to reduce the chance of developing a disease-producing infection from parasites that causes malaria, including species of Plasmodium. e.g. P. falciparum or P. vivax, and the like, and reduce the chance of becoming ill when one is infected, reduce the severity of the illness, such as fever, when one becomes infected, reduce the concentration of parasites in the infected person, or reduce mortality rates from malaria in populations exposed to malaria parasites. In many cases even partial protection or delay in the time it takes an immunized individual as compared to a non-immunized individual to become infected with the parasites or ill from infection is beneficial. Similarly, a vaccine treatment strategy that results in any of these benefits in about 30% of a population may have a significant impact on the health of a community and of the individuals residing in the community. It is generally contemplated that inoculating a subject according to the methods of the invention with genetically attenuated Plasmodium sporozoites of one Plasmodium species will induce protective immunity against challenge with wildtype Plasmodium parasites of the same species. However, it has been shown that immunization with sporozoites of one Plasmodium species can protect against challenge with sporozoites of another Plasmodium species, thus, eliciting cross-species protection in this manner is also within the scope of the invention.

Also provided are methods for prevention of malaria in a subject. The methods comprise administering to the subject a vaccine comprising a genetically attenuated Plasmodium-species vaccine lacking LINUP gene function, which has been prepared aseptically and comprises substantially purified live genetically attenuated Plasmodium sporozoites in an amount effective to prevent malaria.

The prevention and/or treatment of malaria may be readily ascertained by the skilled practitioner by evaluation of clinical or pathological manifestations associated with malarial infection, for example elevated temperature, headache, fatigue, coma, or percent of erythrocytes parasitized. Thus, according to the methods of the present invention, the subject shows improved or absent clinical signs, symptoms or pathological manifestations of malaria following administration of a vaccine comprising purified live attenuated Plasmodium sporozoites.

In some embodiments, the administering step results in infection of a hepatocyte of the subject, and development of the genetically attenuated Plasmodium parasite through liver stage, providing an array of Plasmodium-specific antigens, and the generated immune response ameliorates or protects against infection from a subsequent wildtype Plasmodium challenge. Accordingly, in some embodiments, the disclosed methods confer protective immunity sufficient to reduce or prevent the symptoms of malaria in at least 60% of subjects, at least 70%, at least 80%, at least 90%, at least 95%, or at least 99% of subjects, following exposure to wildtype Plasmodium.

Effective and optimal dosage ranges for vaccines and immunogens can be determined using methods known in the art. Guidance as to appropriate dosages to achieve an anti-malarial effect is provided from the exemplified assays disclosed herein. More specifically, results from the immunization pattern described herein and in cited references can be extrapolated by persons having skill in the requisite art to provide a test vaccination schedule. Volunteer subjects are inoculated with varying dosages at scheduled intervals and test blood samples are evaluated for levels of protection against malaria upon subsequent challenge with infective parasites. Such results can be used to refine an optimized immunization dose and dosage regimen (schedule) for effective immunization of mammalian, specifically human, subjects.

An immune response in a subject can be measured by standard tests including, but not limited to the assessment of humoral and cellular immune responses, including, but not limited to: measurement of antigen specific or parasite stage specific antibody responses: direct measurement of peripheral blood lymphocytes by means known to the art: natural killer cell cytotoxicity assays cell proliferation assays [41] immunoassay's of immune cells and subsets [42, 43] and skin tests for cell mediated immunity [44]. Various methods and analyses for measuring the strength of the immune system have been described, for example, [45].

The vaccines provided comprise aseptic and non-aseptic compositions (preferably aseptic) of purified live attenuated Plasmodium sporozoite substantially free of attendant material, and compositions with a pharmaceutically acceptable diluent, excipient, or carrier. These vaccines are effective in preventing or mitigating malaria upon subsequent challenge with infectious parasites. Methods of formulating pharmaceutical compositions and vaccines are well known to those of ordinary skill in the art [see. e.g., 46].

Comprehended by the invention are vaccine compositions, aseptically prepared or otherwise, comprising purified, live attenuated or non-attenuated Plasmodium sporozoites along with appropriate diluent and buffer. Diluents, commonly Phosphate Buffered Saline (PBS), or Normal Saline (NS), are of various buffer content pH and ionic strength. Such compositions may also include an excipient such as serum albumin, particularly human serum albumin. Serum albumin may be purified from naturally occurring sources such as human blood, or be produced by recombinant DNA or synthesis technologies. Such compositions may also include additives such as anti-oxidants e.g., ascorbic acid, sodium metabisulfite, and/or preservatives or cryopreservatives. Incorporation of the material into particulate preparations of polymeric compounds such as polylactic acid, polyglycolic acid, etc. or into liposomes may also be used. (See. e.g., [46] pages 1435-1712 which are herein incorporated by reference).

In order to determine the effective amount of the vaccines, the ordinary skilled practitioner, considering the therapeutic context, age, and general health of the recipient, will be able to ascertain proper dosing. The selected dosage depends upon the desired therapeutic effect, on the route of administration, and on the duration of the treatment desired. Experiments to determine levels for dosages can be ascertained by one of ordinary skill in the art by appropriate human clinical trials in which various dosage regimens are evaluated for their capacity to elicit protection against malaria.

Disclosed vaccines and disclosed methods of using these vaccines may be useful as one component in a vaccine regimen, each component in turn comprising a discrete vaccine to be administered separately to a subject. Regimens may include sequential immunization with attenuated Plasmodium species sporozoites and other types of Plasmodium vaccines, so-called, prime-boost strategies. This may include attenuated sporozoites as a prime, and Plasmodium-related recombinant protein or proteins in adjuvant as a boost or vice versa. This may also include Plasmodium-related DNA vaccines or a recombinant virus, such as adenovirus, that express Plasmodium-related proteins, as a prime and purified, attenuated sporozoites vaccine as a boost, or vice versa. It may also include sequential or mixed immunization with attenuated Plasmodium species sporozoites and some form of erythrocytic stage parasites, including, killed and live attenuated. A vaccine complex comprising separate components may be referred to as a vaccine regimen, a prime/boost regimen, component vaccine, a component vaccine kit or a component vaccine package, comprising separate vaccine components. For example, a vaccine complex may comprise as a component, a vaccine comprising purified, aseptic, live attenuated sporozoites. The complex may additionally comprise one or more recombinant or synthetic subunit vaccine components, including but not limited to recombinant protein, synthetic polypeptide, DNA encoding these elements per se or functionally incorporated in recombinant virus, recombinant bacteria, or recombinant parasite. A vaccine component may also include aseptic attenuated axenic sporozoites that are allowed to develop to the early liver stage extracellularly.

The following are experimental disclosures of the discovery and analysis of LINUP as a viable target for genetic attenuation, as well as the development of double knockout attenuated Plasmodium.

It is generally noted that the use of the term “or” in the claims is used to mean “and/or” unless explicitly indicated to refer to alternatives only or the alternatives are mutually exclusive, although the disclosure supports a definition that refers to only alternatives and “and/or.”

Following long-standing patent law, the words “a” and “an,” when used in conjunction with the word “comprising” in the claims or specification, denotes one or more, unless specifically noted.

Unless the context clearly requires otherwise, throughout the description and the claims, the words “comprise,” “comprising,” and the like are to be construed in an inclusive sense as opposed to an exclusive or exhaustive sense, such as in the sense of “including, but not limited to.” Words using the singular or plural number also include the plural and singular number, respectively. Additionally, the words “herein,” “above,” and “below,” and words of similar import, when used in this application, shall refer to this application as a whole and not to any particular portions of the application. Words such as “about” and “approximately” imply minor variation around the stated value, usually within a standard margin of error, such as within 10% or 5% of the stated value.

Disclosed are materials, compositions, and components that can be used for, in conjunction with, and in preparation for the disclosed methods and compositions. It is understood that when combinations, subsets, interactions, groups, etc., of these materials are disclosed each of various individual and collective combinations is specifically contemplated, even though specific reference to each and every single combination and permutation of these compounds may not be explicitly disclosed. This concept applies to all aspects of this disclosure including, but not limited to, steps in the described methods. Thus, specific elements of any foregoing embodiments can be combined or substituted for elements in other embodiments. For example, if there are a variety of additional steps that can be performed, it is understood that each of these additional steps can be performed with any specific method steps or combination of method steps of the disclosed methods, and that each such combination or subset of combinations is specifically contemplated and should be considered disclosed. Additionally, it is understood that the embodiments described herein can be implemented using any suitable material such as those described elsewhere herein or as known in the art.

Publications cited herein and the subject matter for which they are cited are hereby specifically incorporated by reference in their entireties.

### EXAMPLES

### Example 1-P. yoelii LINUP is Expressed Exclusively During Liver Stage Development

To identify novel genes giving rise to attenuation only during liver stage development, the late liver stage P. vivax transcriptome that was isolated from the infected livers of human-liver chimeric FRG NOD huHep mice was utilized. Mice were infected with one million P. vivax Thai field strain sporozoites isolated from Anopheles dirus mosquitoes. The mice were euthanized at day 8 after infection, late in liver stage development and the livers were processed for RNA extraction. Probes specific to the P. vivax exome were hybridized to purified RNA, converted to cDNA and bulk sequenced. Reads were normalized and expressed as reads per kilobase of transcript, per one million mapped reads (RPKM). This resulted in the capture of 4397 unique gene transcripts with RPKM values greater than two. To enrich for liver stage specific reads, the P. vivax RPKM reads were expressed as a fold change over the maximal RPKM reads for the orthologous P. falciparum genes expressed during the blood stage of the life cycle. This enabled comparison and contrast of asexual P. vivax liver stage development with asexual P. falciparum blood stage development and specifically isolate genes that were highly expressed only during liver stage development. The top ten genes from this analysis (Table 1) included genes or metabolic pathways already known to be expressed during liver stage development and not during blood stage development.

TABLE 1
Top ten Plasmodium vivax genes expressed during day eight of liver stage development
based on a comparison with Plasmodium falciparum asexual blood stage

| | | | RPKM | | |
|---|---|---|---|---|---|
| | | | Plasmodium vivax | | |
| | | | fold change | | |
| | | | compared to mean | | |
| | | Plasmodium | RPKM for | | |
| Plasmodium | | vivax day 8 | Plasmodium | Plasmodium | |
| vivax gene | | liver stage | falciparum asexual | falciparum | Plasmodium |
| identifier | Gene product | RPKM | blood stage | ortholog | yoelii ortholog |
| PVX_122435 | malonyl CoA-acyl | 857 | 143 | PF3D7_1312000 | PY17X_1412300 |
| | carrier protein | | | | |
| | transacylase | | | | |
| PVX_001010 | conserved hypothetical | 1489 | 68 | PF3D7_0404600 | PY17X_1003700 |
| | protein | | | | |
| PVX_000975 | liver specific protein 2 | 653 | 61 | PF3D7_0405300 | PY17X_1004400 |
| | (LISP2) | | | | |
| PVX_085550 | liver specific protein 1 | 717 | 54 | PF3D7_1418100 | PY17X_1027000 |
| | (LISP1) | | | | |
| PVX_101370 | conserved hypothetical | 375 | 53 | PF3D7_1249700 | PY17X_1465200 |
| | protein | | | | |
| PVX_083460 | conserved hypothetical | 86 | 42 | PF3D7_1351300 | PY17X_1369800 |
| | protein | | | | |
| PVX_088860 | sporozoite invasion- | 259 | 37 | PF3D7_0830300 | no ortholog |
| | associated protein 2 | | | | |
| | (SIAP2) | | | | |
| PVX_111315 | protoporphyrinogen | 110 | 29 | PF3D7_1028100 | PY17X_0513300 |
| | oxidase | | | | |
| PVX_086345 | conserved hypothetical | 71 | 28 | PF3D7_1401700 | no ortholog |
| | protein | | | | |
| PVX_113280 | liver merozoite | 103 | 25 | PF3D7_0602300 | PY17X_0102700 |
| | formation protein | | | | |
| | (PALM) | | | | |

Known genes included LISP1 (30), LISP2 (31-33), PALM (34) and SIAP2 (35). Malonyl CoA-acyl carrier protein transacylase was also detected, a precursor of type II fatty acid biosynthesis, a pathway known to be expressed during P. falciparum liver stage development and critical for P. yoelii and P. berghei liver stage development (36-39). This result gave us assurance that our methodology for the discovery of liver stage specific transcripts was sound. We also had a requirement that orthologs were present in the P. yoelii genome so we could use this rodent malaria model to efficiently analyze spatial and temporal expression as well as essentiality. Of the remaining five genes in the top ten, four had orthologs in the P. yoelii genome and included a putative protoporphyrinogen oxidase, PY17X_0513300, and three hypothetical genes, PY17X_1003700, PY17X_1465200 and PY17X_1369800. Analysis using PlasmoDB of the P. yoelii genes and their P. falciparum orthologs for both protein expression (P. yoelii and P. falciparum) and essentiality in the blood stage (P. falciparum) suggested that although PY17X_051300/PF3D7_102800 was dispensable in P. falciparum asexual blood stages, the protein was expressed in the P. falciparum blood stage gametocyte and the P. yoelii oocyst sporozoite and thus the gene was not studied further. Analysis of PY17X 1369800/PF3D7_1351300 suggested the gene was essential for P. falciparum asexual blood stage replication and thus not studied further. Analysis of

PY17X_1003700/PF3D7_0404600 suggested the gene was dispensable in P. falciparum asexual blood stages, but the protein was expressed in both the P. falciparum blood stage merozoite and sporozoite and thus the gene was not studied further. Of note, analysis of PY17X 1465200/PF3D7_1249700 suggested the gene was dispensable for P. falciparum asexual blood stage replication. Further, there was no evidence of protein expression in P. falciparum and expression in the liver stage of P. yoelii at 40 hours of development. Therefore, PY17X_1465200/PF3D7_1249700 underwent further study.

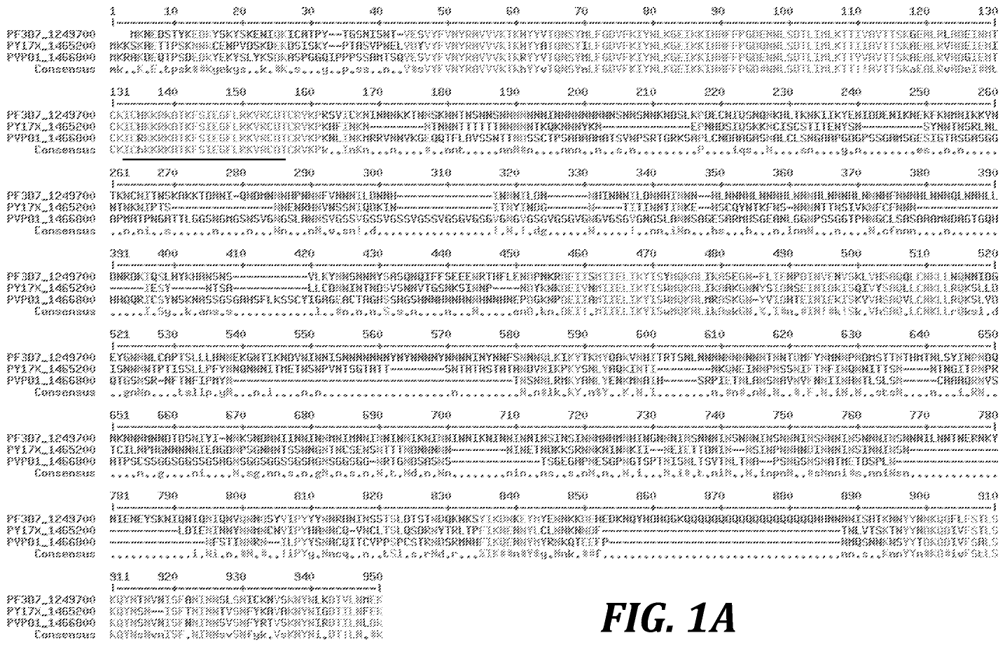

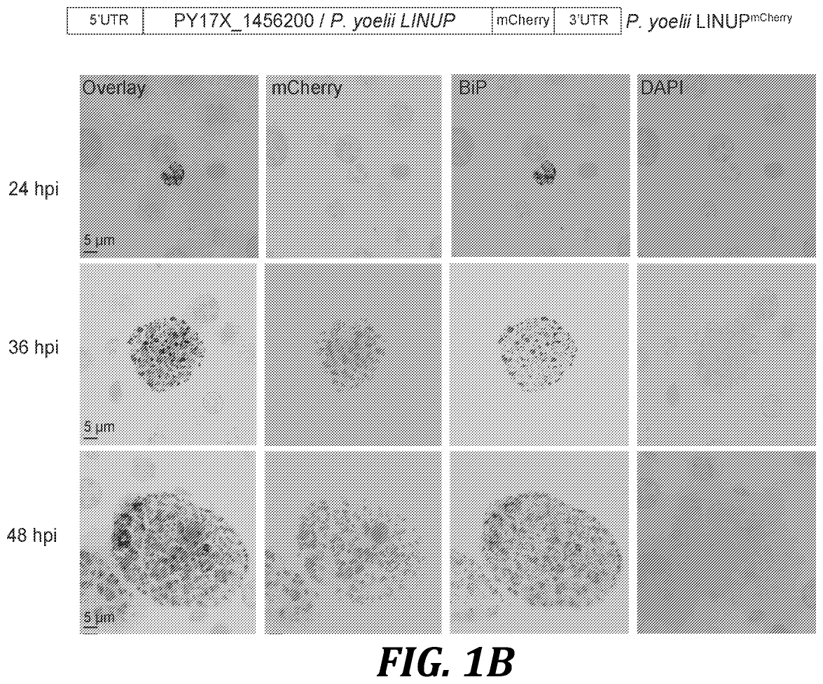

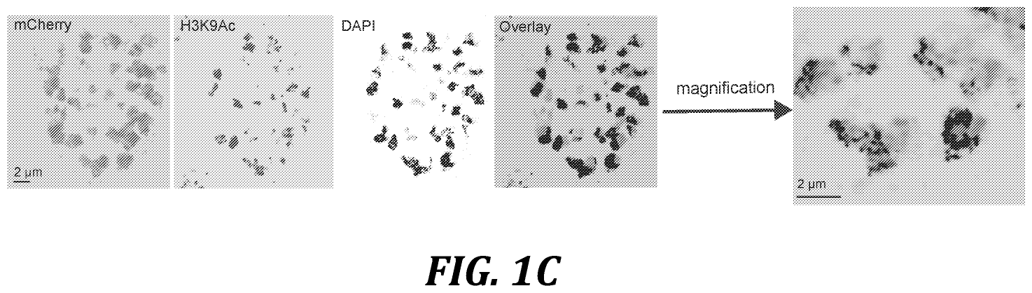

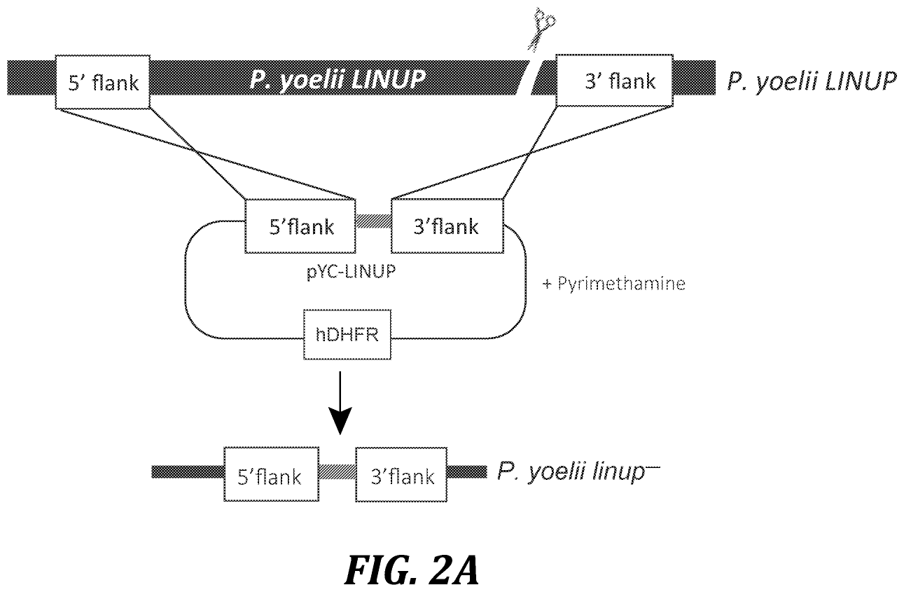

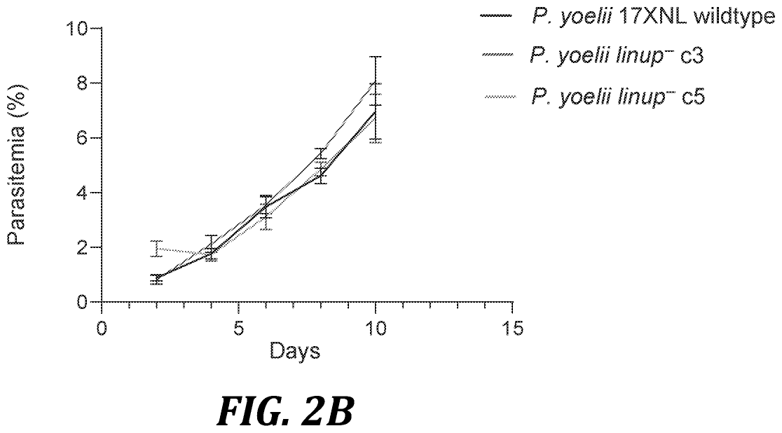

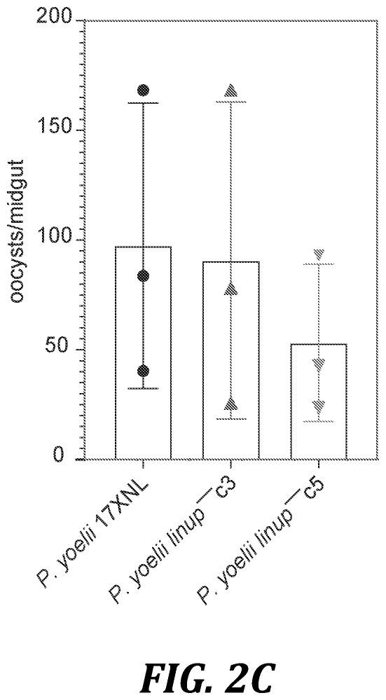

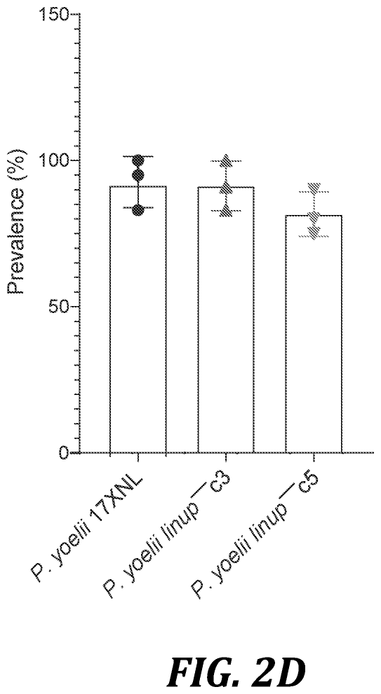

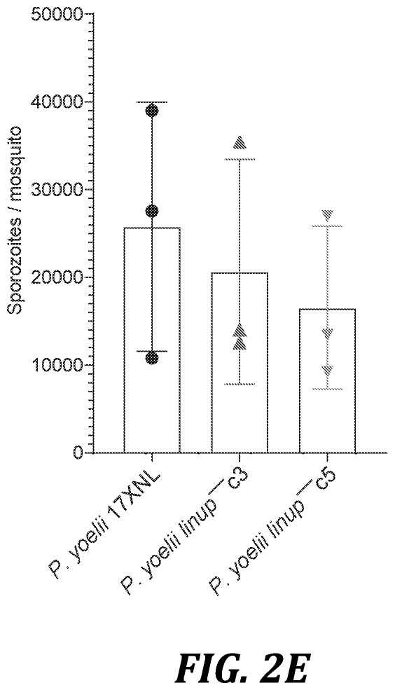

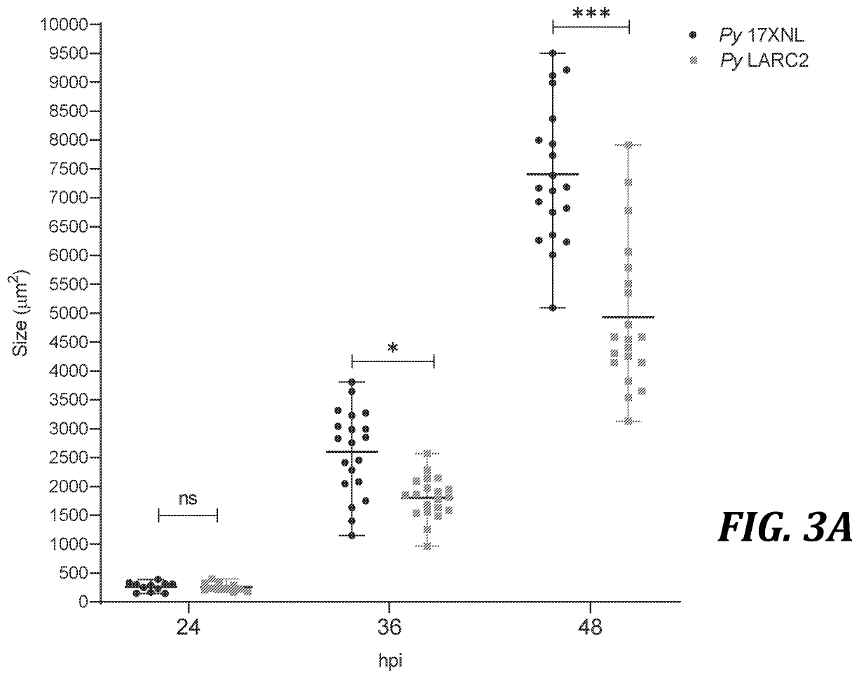

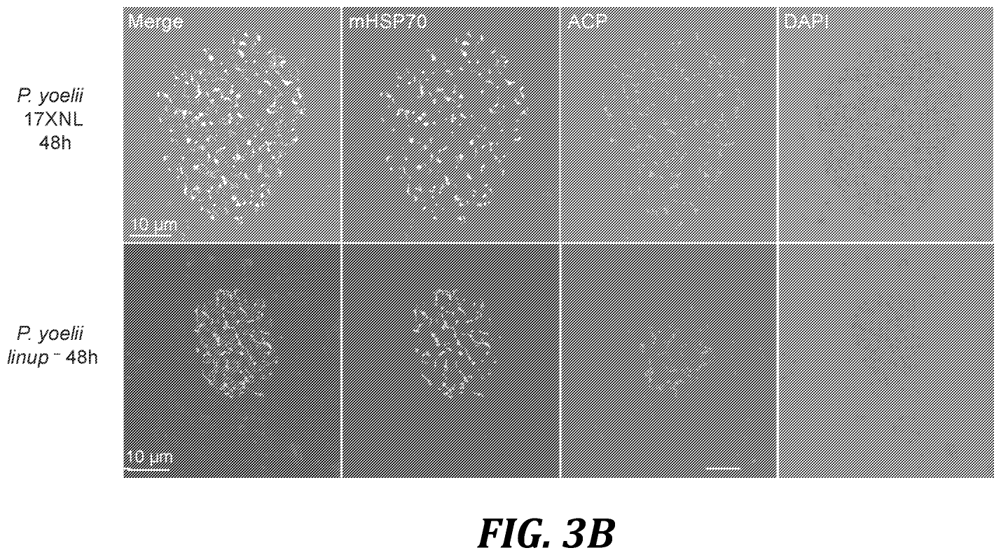

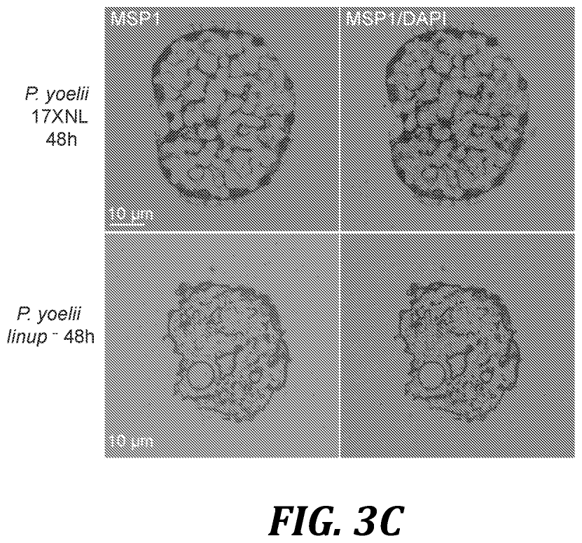

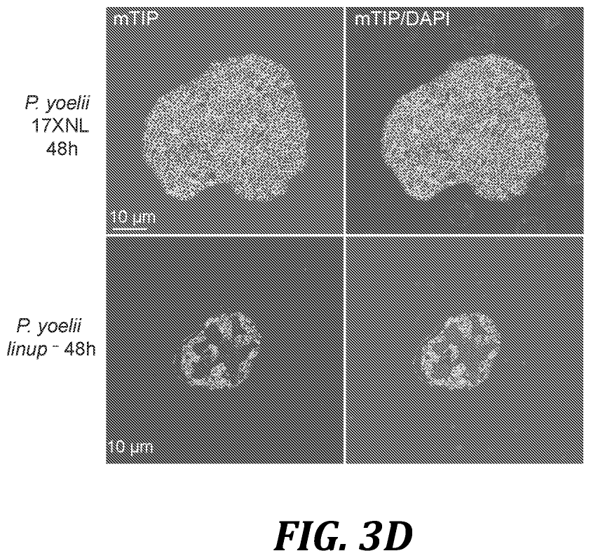

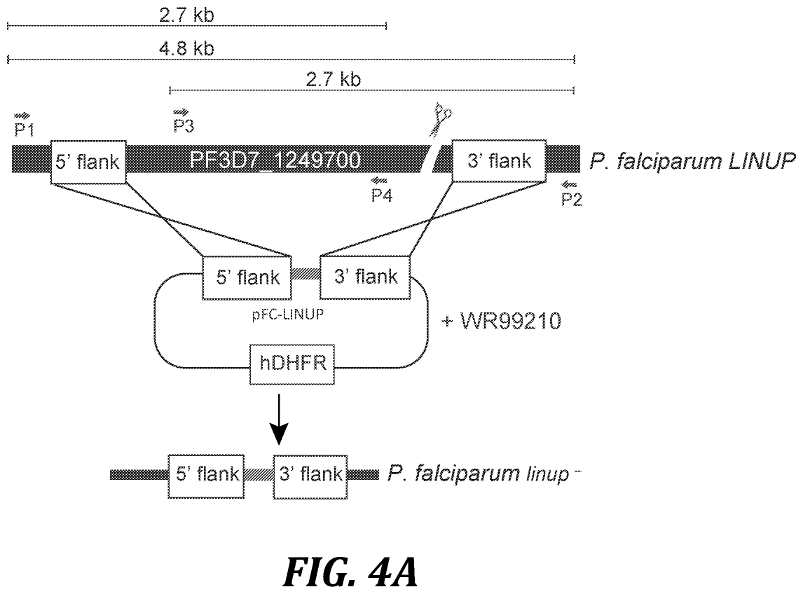

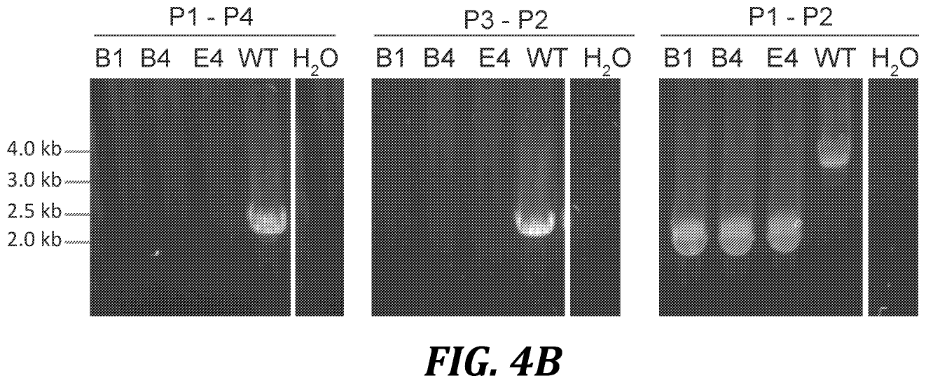

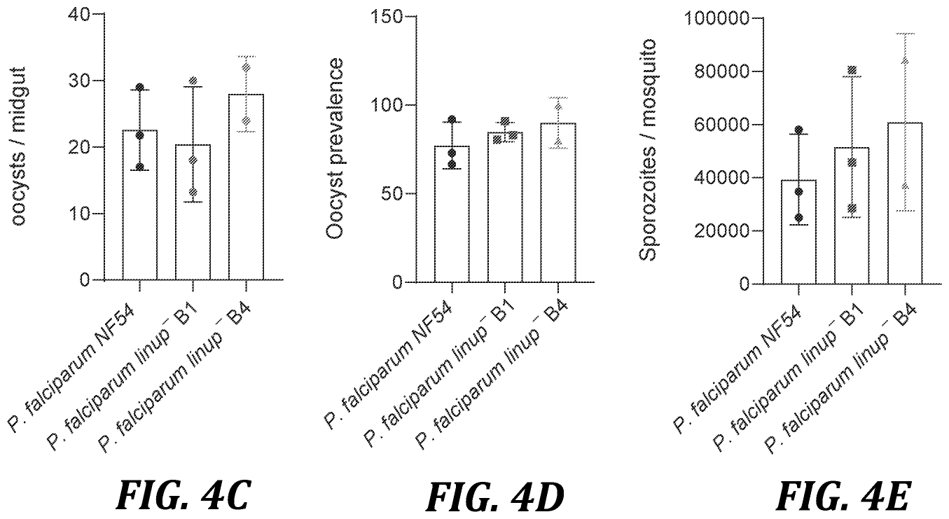

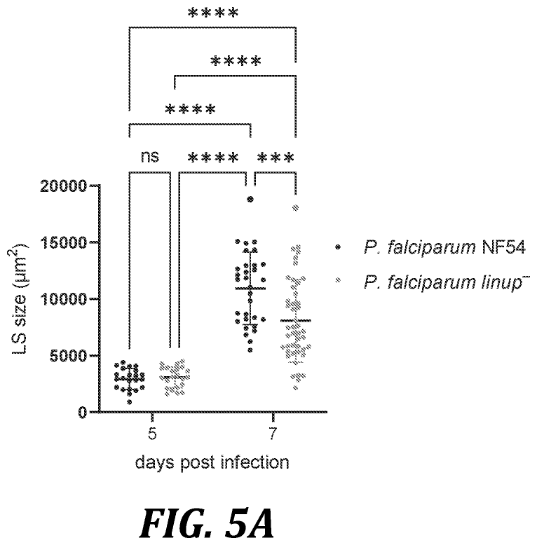

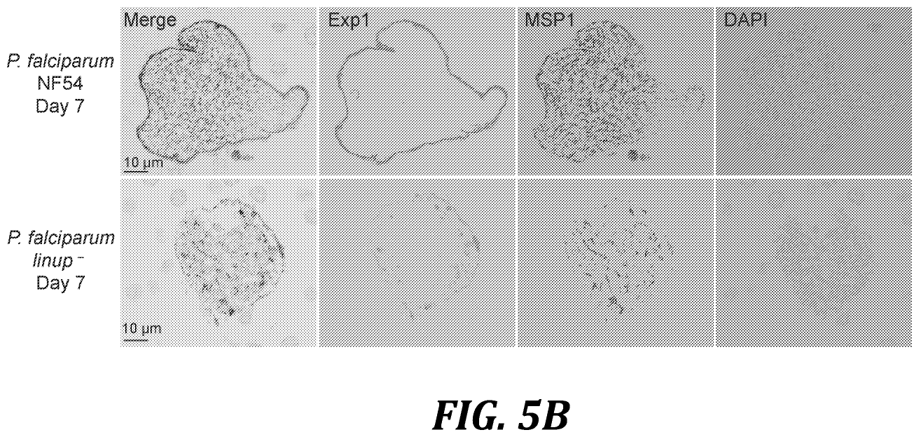

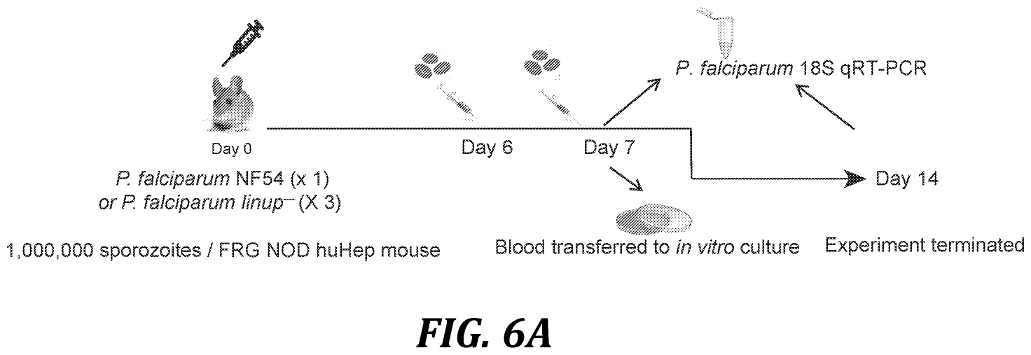

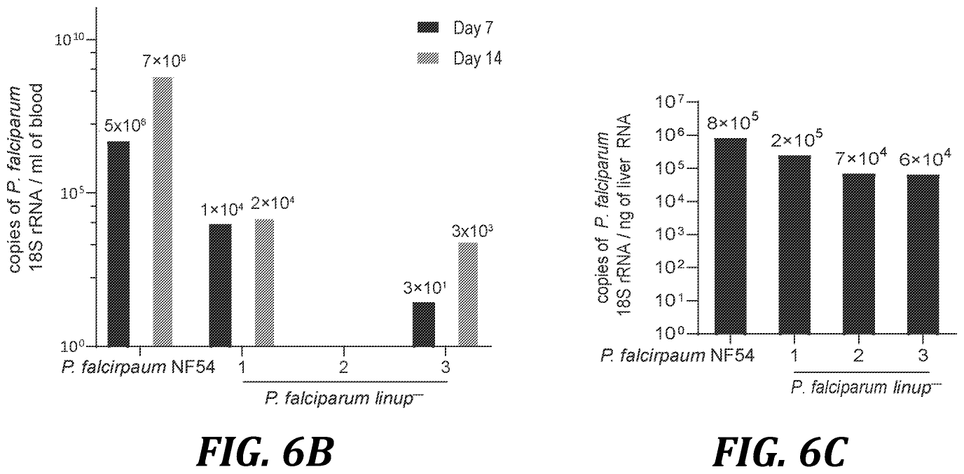

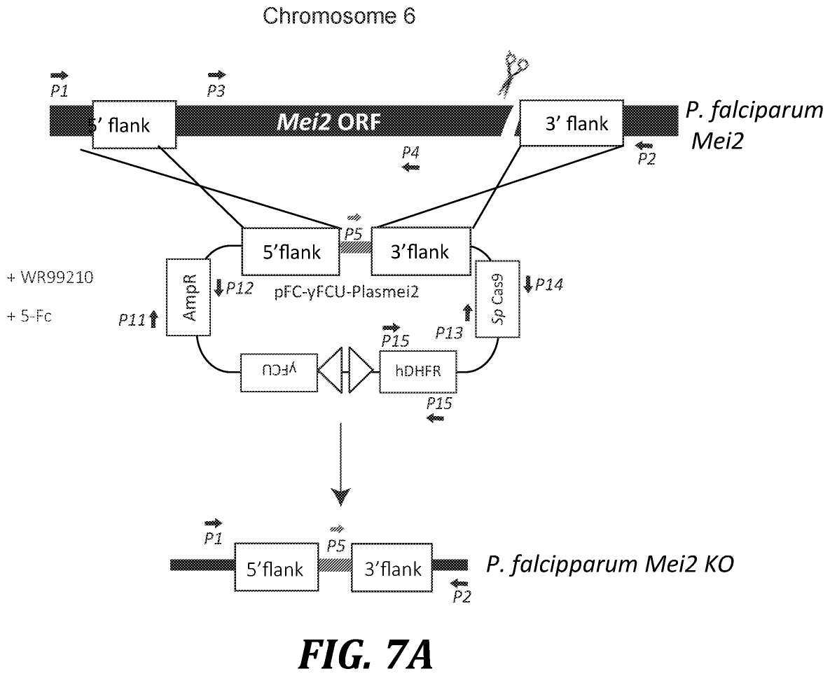

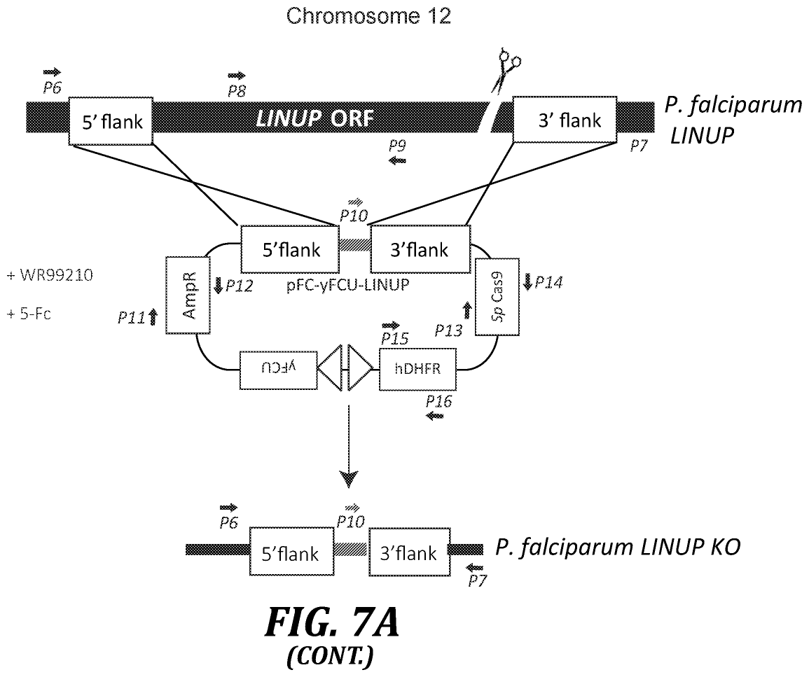

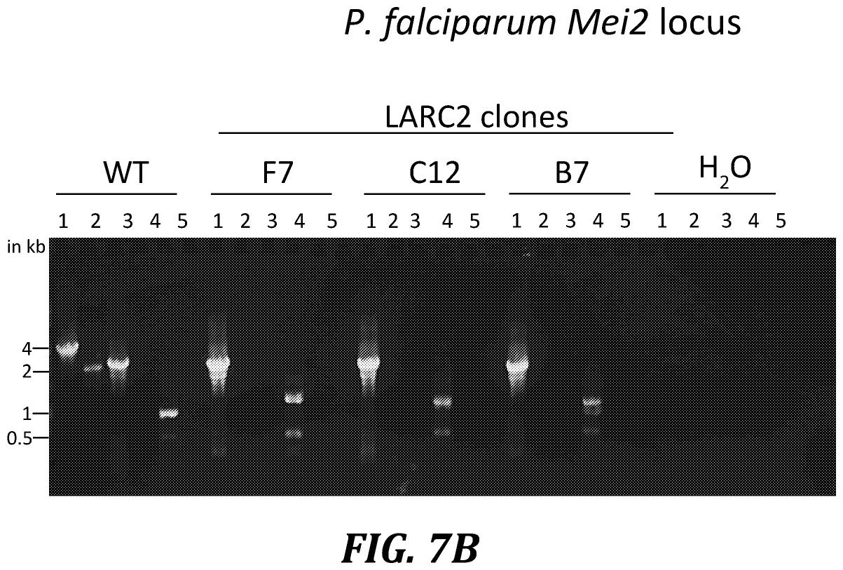

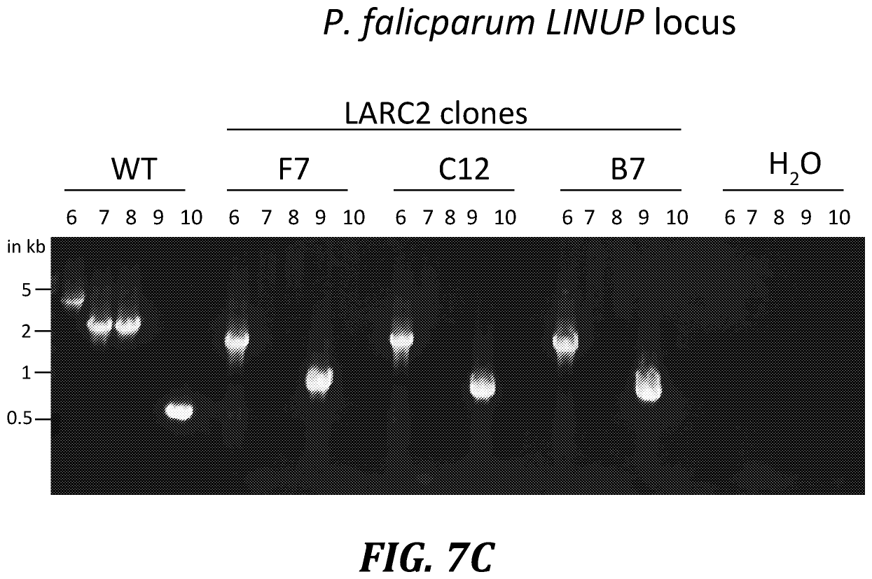

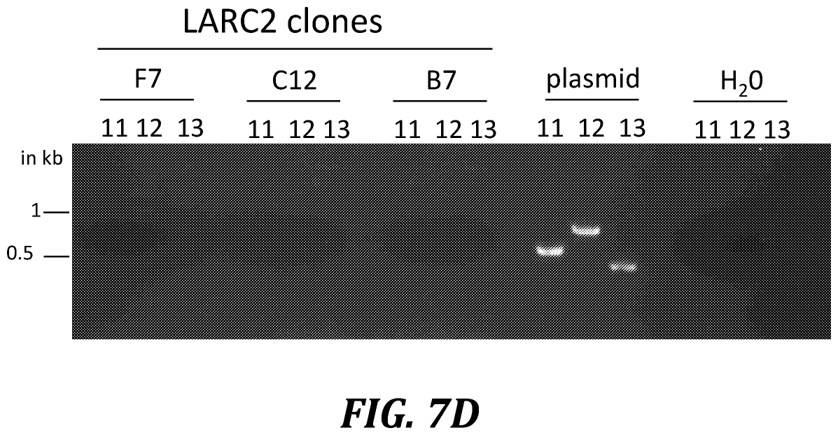

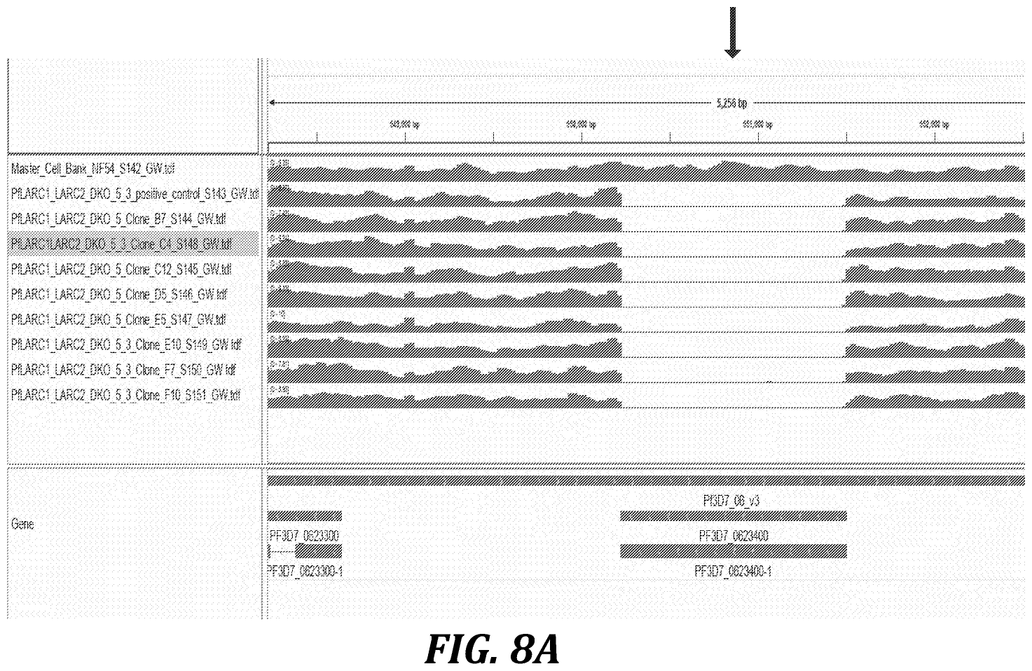

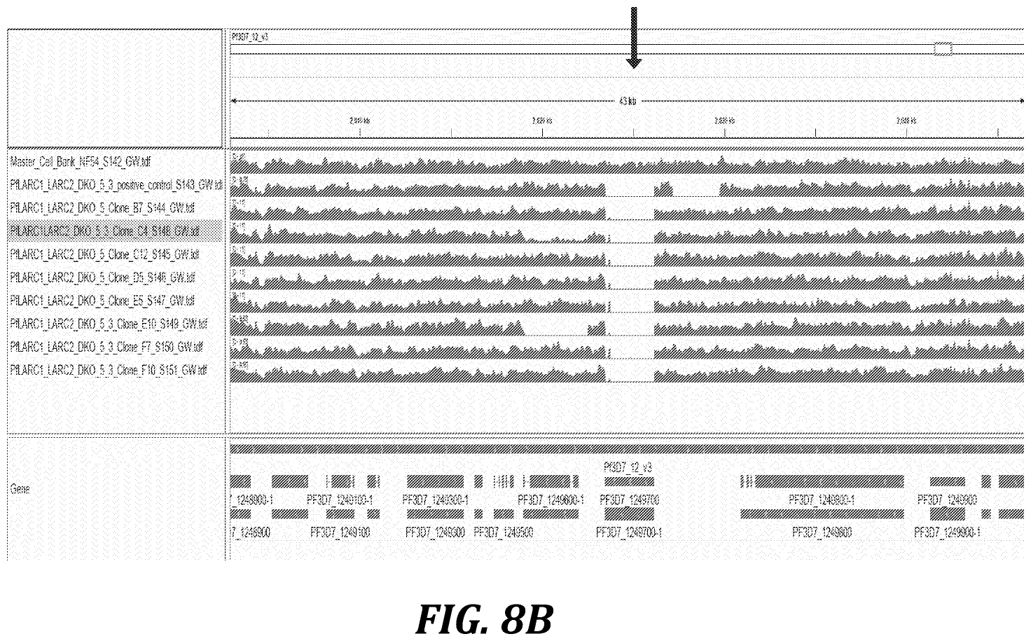

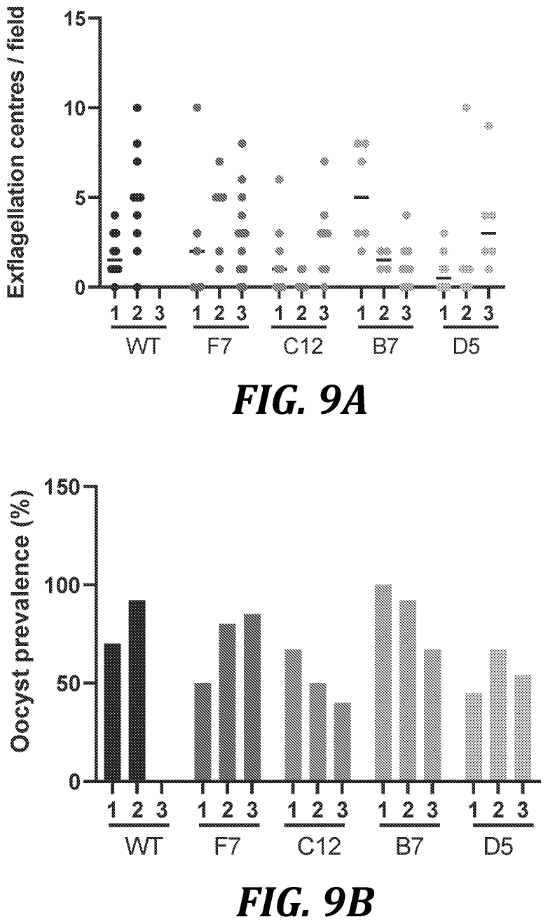

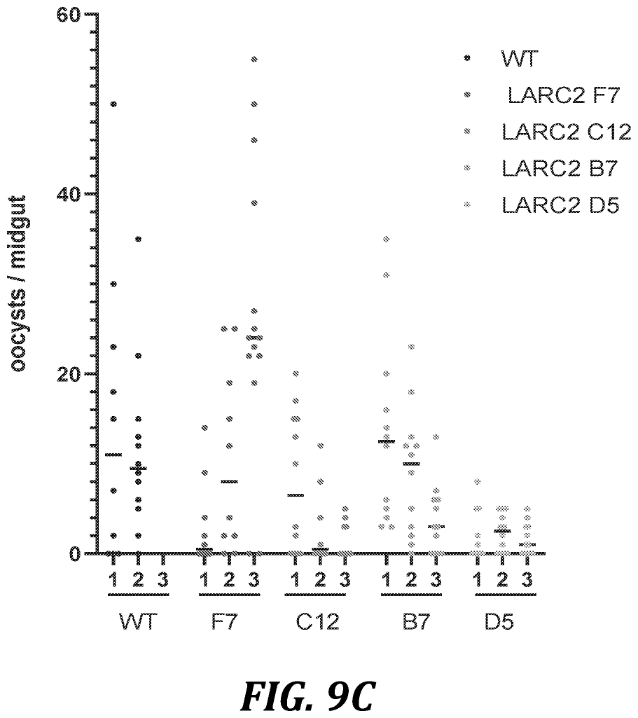

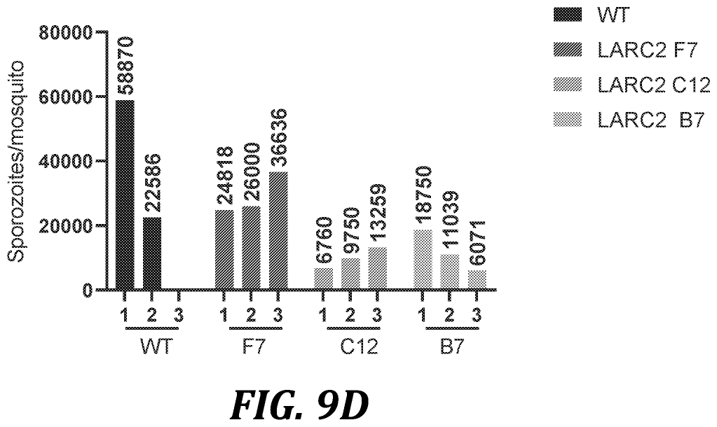

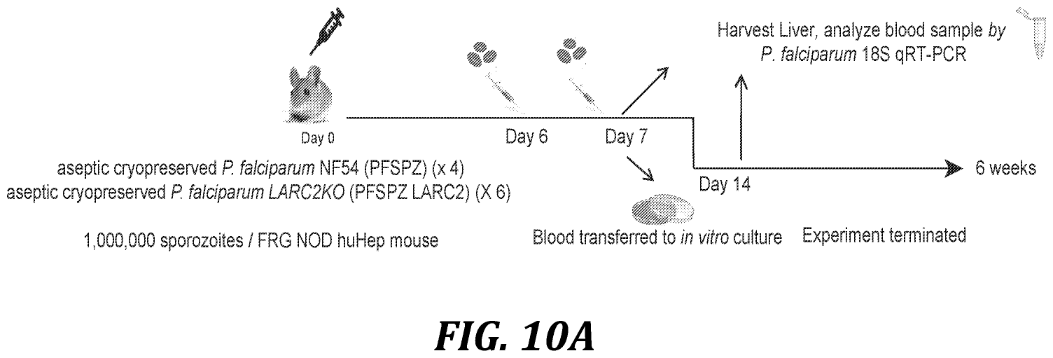

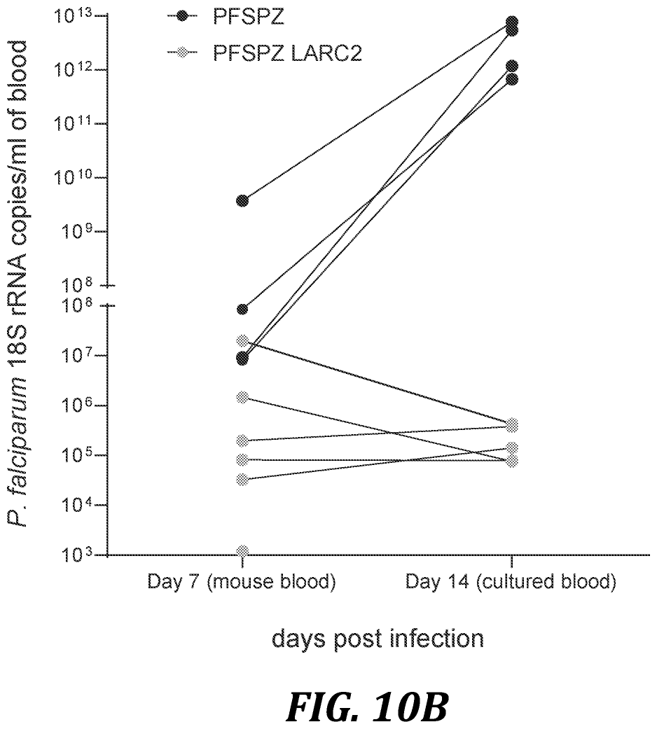

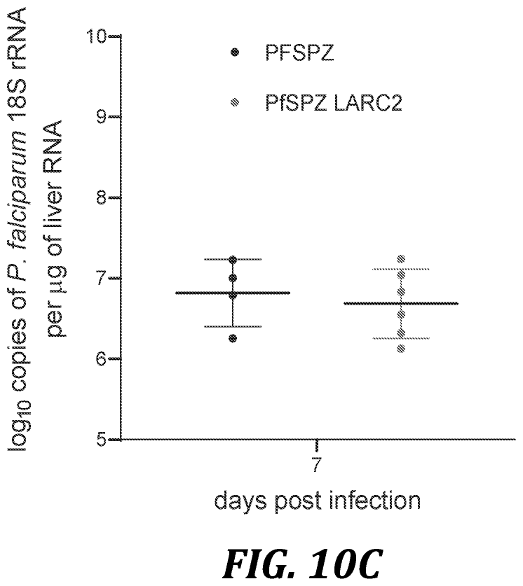

PY17X_1465200 is a single exon gene coding for a 746 amino acid protein with no known protein features and is conserved across Plasmodium species (FIG. 1A). The overall amino acid identity between the P. yoelii and P. falciparum and P. vivax syntenic orthologs is 40%, whilst amino acid similarity was 60%. Identity in a 122 amino acid stretch near to the N-terminus (amino acids 44-161) was 89%. In addition, the gene has no orthologs in other Apicomplexa or any other eukaryote and is thus unique to Plasmodium. To determine the spatial-temporal expression pattern of PY17X_1465200, a transgenic P. yoelii parasite was created with an mCherry tag attached to the C-terminus of the endogenous PY17X_1465200 (FIG. 1B, top panel cartoon). Standard transfection procedures resulted successful double crossover homologous recombination and the transgenic parasite was cloned and integration of the mCherry tag was confirmed by transgene specific PCR. Comparison of PY17X_1465200 mCherry tagged parasite clones with wildtype parasites did not reveal statistically significant differences in life cycle progression. Specifically, the mosquito stage and liver stage of the life cycle were comparable since the intravenous injection of 10,000 salivary gland sporozoites from both wildtype and tagged parasites into groups of Swiss Webster mice, all mice demonstrated blood stage infection on the third day after sporozoite injection, based on Giemsa-stained thin blood smears. With the knowledge that the tagged parasite completed the life cycle normally, expression of the mCherry tag was monitored across the complete life cycle by immunofluorescent assay (IFA) of infected tissue. Interestingly, mCherry expression was only seen during liver stage development which in P. yoelii has an approximate 52 hour duration. Expression was not detected at 24 hours (FIG. 1B) but was detected at both 36 and 48 hours (FIG. 1B), a timepoint where exo-erythrocytic merozoites are forming. Expression was punctate and did not overlap with the punctate expression of the endoplasmic reticulum marker BiP (FIG. 1B). However, expression did partially overlap with the nuclear stain DAPI as well as with the active histone marker, histone 3, acetylated lysine 9 (FIG. 1C). Thus, we concluded that PY17X_1465200 has a pattern of nuclear expression only during mid-to late-liver stage development. We therefore named the gene and its protein product LINUP, liver stage nuclear protein, and the tagged parasite P. yoelii LINUP^{mCherry}. Further analysis of the LINUP amino acid sequence revealed a conserved N-terminal 25 amino acid nuclear localization sequence (NLS) in P. yoelii, P. falciparum and P. vivax that was almost identical between across the three species (FIG. 1A) The presence of the NLS explains the nuclear localization of P. yoelii LINUP. To further study the importance of LINUP in liver stage development, a gene knockout approach was used.

### Example 2-P. yoelii LINUP is Critical for Liver Stage Development

To study the essentiality of LINUP expression on P. yoelii liver stage development, we used CRISPR/Cas9 technology to delete the LINUP gene from the P. yoelii genome (FIG. 2A). After schizont transfection, in vivo positive drug selection with pyrimethamine in mice, and isolation of drug resistant parasites, gene knockout was confirmed by PCR on isolated genomic DNA, using specific primers.

TABLE 2
The combination of primers used to verify the gene deletion
of P. falciparum Mei2 and P. falciparum LINUP in P. falciparum
LARC2KO clones are indicated. The sizes of the PCR products
are indicated in kilobases (kb). See also FIG. 7.

| | Primer | WT | Knockout | Plasmid |
|---|---|---|---|---|
| 1 | P1-P2 | 3.5 kb | 2.3 kb | — |
| 2 | P1-P4 | 2.1 kb | — | — |
| 3 | P3-P2 | 2.4 kb | — | — |
| 4 | P5-P2 | — | 1.3 kb | — |
| 5 | P3-P4 | 1.0 kb | — | — |
| 6 | P6-P7 | 4.7 kb | 2.0 kb | — |
| 7 | P6-P9 | 2.7 kb | — | — |
| 8 | P8-P7 | 2.6 kb | — | — |
| 9 | P10-P7 | — | 1.0 kb | — |
| 10 | P8-P9 | 0.6 kb | — | — |
| 11 | P11-P12 | — | — | 0.5 kb |
| 12 | P13-P14 | — | — | 0.7 kb |
| 13 | P15-P16 | — | — | 0.5 kb |

P. yoelii linup⁻ parasites were cloned, and two clones, c3 and c5, were initially used for downstream phenotypic analysis. The two clones were initially compared to the wildtype parent during blood stage growth (FIG. 2B) and the mosquito stages of development and there was no statistical difference in overall oocyst numbers (FIG. 2C) as well as oocyst prevalence (FIG. 2D) and salivary gland sporozoites per mosquito (FIG. 2E) in biological replicate experiments, suggesting that deletion of LINUP did not affect the mosquito stage of the life cycle. This was expected since expression of LINUP was not seen until the mid-to-late liver stage.

To determine if liver stage development was affected by the deletion of LINUP, P. yoelii wildtype and linup⁻ sporozoites were isolated from infected Anopheles stephensi mosquito salivary glands and injected intravenously into groups of laboratory mice including outbred Swiss Webster mice as well as the susceptible inbred strains BALB/cJ and BALB/cByJ (Table 3).

TABLE 3
Attenuation of P. yoelii linup⁻ liver stage development in Swiss Webster and BALB/c mice.

| | | | | Number of mice | |
|---|---|---|---|---|---|
| Parasite | | Mouse | Number of | patent/number of mice | |
| genotype | Clone^{a} | strain^{b} | sporozoites^{c} | infected^{d} | Days to patency (number of mice)^{e} |
| P. yoelii | N/A | BALB/cJ | 1,000 | 5/5 | 4 |
| 17XNL | | | 10,000 | 5/5 | 3(2) |
| | | | | | 4(3) |
| | | | 50,000 | 3/3 | 3 |
| | | BALB/cByJ | 50,000 | 3/3 | 3 |
| | | SW | 50,000 | 5/5 | 3 |
| P. yoelii | c3 | BALB/cJ | 1,000 | 1/10 | 12 |
| linup⁻ | | | 10,000 | 7/20 | 7(1) |
| | | | | | 9(2) |
| | | | | | 10(1) |
| | | | | | 12(3) |
| | | | 50,000 | 5/10 | 7(2) |
| | | | | | 8(1) |
| | | | | | 9(2) |
| | | BALB/cByJ | 50,000 | 9/20 | 8(3) |
| | | | | | 9(2) |
| | | | | | 10(4) |
| | | SW | 50,000 | 9/10 | 8(2) |
| | | | | | 10(7) |
| | c5 | SW | 50,000 | 5/5 | 7(2) |
| | | | | | 9(2) |
| | | | | | 10(1) |

^{a}Two independently generated clones of Py linup⁻ (c3 and c5) were used for analysis.
^{b}The strain of mice used for the analysis is indicated. SW: Swiss Webster.
^{c}Salivary gland sporozoites were isolated from infected Anopheles stephensi mosquitoes, and mice were challenged intravenously with the listed number of sporozoites.
^{d}The number of patent mice per number of mice challenged is indicated. Detection of blood stage patent parasitemia was carrier out by Giemsa-stained thin blood smear. Attenuation was considered if mice remained blood stage negative for 21 days.
^{e}If mice became blood stage patent, the day to patency is listed, with the number of mice that became patent in parentheses.

The time to blood stage infection, in days, was then determined from Giemsa-stained thin blood smears. All Swiss Webster mice infected with 50,000 wildtype sporozoites all demonstrated blood stage infection on days three whereas nine of ten mice demonstrated blood stage infection when infected with P. yoelii linup⁻ sporozoites and there was a severe delay to patency with mice becoming infected on days eight and ten after infection, suggesting that P. yoelii linup⁻ liver stage are severely attenuated (Table 3). Similarly, BALB/cByJ mice infected with 50,000 wildtype sporozoites all demonstrated blood stage infection on day three whereas only nine of twenty mice infected with P. yoelii linup⁻ sporozoites demonstrated blood stage infection and the day to infection ranged from days eight through ten (Table 3). A dose escalation study in BALB/cJ mice revealed further evidence of liver stage attenuation. Mice were infected with 1,000, 10,000 and 50,000 sporozoites. Mice infected with wildtype sporozoites all demonstrated blood stage infection on day four (1,000), days three-four (10,000) and day three (50,000) whereas for P. yoelii linup⁻ sporozoite infection only one of ten mice demonstrated blood stage infection (1,000) on day twelve, seven of twenty demonstrated blood stage infection (10,000) on days seven through twelve, and five of ten became patent (50,000) on days seven through nine (Table 3). Thus, P. yoelii linup⁻ parasites are severely attenuated during liver stage development.

### Example 3-P. yoelii Linup⁻ Liver Stage Growth is Reduced and Exo-Erythrocytic Merozoites Formation is Attenuated

To further study the attenuation of the P. yoelii linup⁻ liver stage, BALB/cByJ mice were infected intravenously with 250,000 P. yoelii linup⁻ sporozoites. Mice were euthanized at time points after infection (24, 36 and 48 hours), livers were removed, perfused, fixed, sliced and subjected to IFA to determine both size and protein/DNA expression patters in comparison to a wildtype infection (FIG. 3). At 24 hours, P. yoelii linup⁻ liver stages appeared to be comparable in size to wildtype (FIG. 3A). However, at 36 hours, P. yoelii linup⁻ liver stages were statistically smaller than wildtype (FIG. 3A). The decreased size was more pronounced at 48 hours (FIG. 3A) and at this time point, clear differences in protein/DNA expression were also seen. At 48 hours, the branching of the mitochondria and apicoplast was reduced in the P. yoelii linup⁻ liver stage as compared to the wildtype (FIG. 3B). In addition, the expression pattern of the parasite plasma membrane marker MSP1, which delineates exo-erythrocytic merozoite was also aberrant (FIG. 3C), showing incomplete cytomere formation. Furthermore, expression of MTIP, part of the inner membrane complex of fully mature merozoites was seen in 17% of wildtype liver stages but only 7% of P. yoelii linup⁻ liver stages, and its expression was aberrant in those P. yoelii linup⁻ liver stages that showed MTIP expression (FIG. 3D). There was also no evidence of the massive nuclear division that takes place during the formation of exo-erythrocytic merozoites in the P. yoelii linup⁻ liver stages. Nevertheless, merozoites can be released from P. yoelii linup⁻ liver stages since some mice showed liver stage infection after P. yoelii linup⁻ sporozoite injection. Thus, in a minority of cases, viable exo-erythrocytic merozoites can be formed.

### Example 4-Immunization of Mice with P. yoelii Linup⁻ Sporozoites Protects from a Wildtype Challenge

Rodent malaria GAP that arrest during liver stage development are powerful immunogens that can protect from a wildtype sporozoite challenge [26-28]. To determine if immunization of susceptible BALC/cJ mice with P. yoelii linup⁻ sporozoites could protect from challenge groups of mice were intravenously immunized with either 1,000 or 10,000 P. yoelii linup⁻ sporozoites or mock immunized with an equivalent volume of salivary gland extract from uninfected mosquitoes (Table 4). Only mice that did not demonstrate liver stage infection after P. yoelii linup⁻ sporozoite infection were ultimately challenged with wildtype sporozoites. Mice were immunized twice, approximately 33 day's apart and then challenged after a further 34 days with an intravenous injection of 10,000 wildtype sporozoites. Mice immunized with salivary gland extract all became blood stage infected three-four days after challenge (Table 4).

TABLE 4
P. yoelii linup⁻ immunization protects from a wildtype sporozoite challenge

| | Number of sporozoites used for immunization/challenge | | | | Number of mice | |
|---|---|---|---|---|---|---|
| | | Boost (days after | | Challenge (days after | protected (days to | |
| Parasite genotype | Prime^{a} | prime)^{a} | | boost)^{b} | patency)^{c} | |
| N/A | Salivary gland | Salivary gland | | 10,000 | 0/5 | (3, 4) |
| | extract | extract | | | | |
| P. yoelii linup⁻ | 1,000 | 1,000 | (33) | 10,000 (34) | 6/9 | (8, 10) |

| | | | | | |
|---|---|---|---|---|---|
| P. yoelii linup⁻ | 10,000 | 10,000 | (33) | 10,000 (34) | 13/13 |

^{a}P. yoelii linup⁻ salivary gland sporozoites were isolated from infected Anopheles stephensi mosquitoes, and mice were immunized intravenously with the listed number of sporozoites. The days after the prime that the boosts took place is indicated in parentheses.
^{b}Mice were challenged intravenously with wildtype salivary gland sporozoites. The days after the boost the challenge took place are indicated in parentheses.
^{c}The number of protected mice per number of mice challenged is indicated and the days to patency are indicated in parentheses. Protection was considered complete if mice remained blood stage negative for 21 days after challenge, based on Giemsa-stained thin blood smear.

Six of nine mice immunized twice with 1,000 P. yoelii linup⁻ sporozoites were protected from challenge and the remaining three mice showed a significant delay to blood stage infection, becoming infected on days eight to ten after challenge (Table 4). All mice (thirteen) immunized twice with 10,000 P. yoelii linup⁻ sporozoites were protected after challenge. This result demonstrates that immunization with P. yoelii linup⁻ GAP engenders a highly effective immune response that protects mice from a significant wildtype sporozoite challenge.

### Example 5-Phenotypic Breakthrough Analysis of P. falciparum Linup⁻ Parasites in FRG NOD huHep Mice

P. falciparum NF54 and P. falciparum linup⁻ sporozoites were isolated from salivary glands of infected Anopheles stephensi mosquitoes. For analysis of P. falciparum linup⁻ liver stage, 1×10⁶ P. falciparum NF54 and 1×10⁶ P. falciparum linup⁻ sporozoites were injected intravenously (retro-orbital) into four FRG NOD huHep mice per group. Livers were harvested on days 5 and 7 and used for IFA (FIG. 6A). To evaluate for blood stage transition of P. falciparum linup⁻ in FRG NOD huHep mice, 1×10⁶ P. falciparum NF54 and P. falciparum linup⁻ sporozoites from clone were injected intravenously into one and three FRG NOD huHep mice per group respectively. On day 6 and 7, 400 μl of 70% RBCs were injected intravenously to enable transition of liver stage parasites to blood. Four hours after human RBC repopulation on day 7, mice were euthanized, blood was collected by cardiac puncture and 50 μl of blood from each mouse was used for qRT-PCR analysis to detect parasite 18S RNA. Blood was washed three times in asexual media, a volume of human RBCs equal to the packed RBC volume was added and blood was transferred to in vitro culture. Fresh media was replaced daily, and cultures were analyzed every 2-3 days by thick smear for presence of parasites for up to 10 days. Samples from in vitro culture were analyzed for presence of 18S rRNA by qRT-PCR after 10 days.

The values for the three mice injected with P. falciparum linup⁻ sporozoites ranged from 0 to 1×10⁴, demonstrating a severe attenuation in the transition (FIG. 6B). Blood from the exsanguinated animals was placed into in vitro culture and sampled seven days later, day 14 after sporozoite injection. P. falciparum NF54 wildtype parasite replication during this time was significant giving rise to 7×10⁸ copies of 18S rRNA per ml of blood, a greater then 100-fold increase (FIG. 6B) and parasites were present by assessment of Giemsa-stained thin blood smears. In contrast, culturing the blood from mice injected with P. falciparum linup⁻ sporozoites showed no significant increase in parasite biomass, 0 to 2×10⁴ copies of 18S rRNA per ml of blood (FIG. 6B). This result suggests that the initial quantification of parasite biomass in blood from mice injected with P. falciparum linup⁻ sporozoites was due to the release of parasite rRNA from attenuated and non-viable liver stage parasites into the blood stream of the mice and not viable exo-erythrocytic merozoites. To further assess P. falciparum linup⁻ liver stage attenuation, parasite biomass, measured as copies of 18S rRNA per ng of liver RNA was determined in the mice used for the transition experiment (FIG. 6C). This suggested that with P. falciparum linup⁻ liver stage biomass was reduced as compared to the wildtype and is likely due to the aberrant maturation of exo-erythrocytic merozoites seen by IFA (FIG. 5B).

### Example 6-Phenotypic Breakthrough Analysis of P. falciparum LARC2 Parasites in FRG NOD huHep Mice

P. falciparum NF54 and P. falciparum LARC2 sporozoites were isolated from salivary glands of infected Anopheles stephensi mosquitoes. To evaluate for blood stage transition of P. falciparum LARC2 in FRG NOD huHep mice, 1×10⁶ aseptic cryopreserved P. falciparum NF54 (PFSPZ) and aseptic cryopreserved P. falciparum LARC2 (PFSPZ LARC2) sporozoites were injected intravenously into four and six FRG NOD huHep mice per group respectively (FIG. 10A). On day 6 and 7, 400 μl of 70% RBCs were injected intravenously to enable transition of liver stage parasites to blood. Four hours after human RBC repopulation on day 7, mice were euthanized, blood was collected by cardiac puncture and 50 μl of blood from each mouse was used for qRT-PCR analysis to detect parasite 18S RNA. Blood was washed three times in asexual media, a volume of human RBCs equal to the packed RBC volume was added and blood was transferred to in vitro culture. Fresh media was replaced daily, and cultures were analyzed every 2-3 days by thick smear for presence of parasites for up to 6 weeks.

On day 7, parasite densities for mice infected with PfSPZ ranged between 10³-10⁶ parasite equivalents/ml by P. falciparum 18S qRT-PCR (FIG. 10B and Table 5).

TABLE 5
P. falciparum LARC liver stages do not transmit
to productive blood stage infection after sporozoite
infection in FRG NOD huHep mice repopulated with human
RBCs and do not grown out in in vitro culture.

| | | Blood stage | | |
|---|---|---|---|---|
| | | parasites by | | |
| | | Giemsa smear | Copies of Pf | Parasite |
| | | (days post | 18S rRNA/ml | equivalents/ml |
| Mouse¹ | Strain | transition) | on day 7 | on day 7 |
| PFSPZ_1 | Pf SPZ | Positive (day 2 | 85,231,282 | 11,518 |
| | | after transition) | | |
| PFSPZ_2 | Pf SPZ | Positive (day 3 | 8,147,908 | 1,101 |
| | | after transition) | | |
| PFSPZ_3 | Pf SPZ | Positive (day 2 | 3,751,204,288 | 506,919 |
| | | after transition) | | |
| PFSPZ_4 | Pf SPZ | Positive (day 1 | 9,236,016 | 1,248 |
| | | after transition) | | |
| PFSPZ | LARC2 | Negative after | 1,199 | 0 |
| LARC2_1 | | 6 weeks | | |
| PFSPZ | LARC2 | Negative after | 1,455,700 | 197 |
| LARC2_2 | | 6 weeks | | |
| PFSPZ | LARC2 | Negative after | 198,041 | 27 |
| LARC2_3 | | 6 weeks | | |
| PFSPZ | LARC2 | Negative after | 32,655 | 4 |
| LARC2_4 | | 6 weeks | | |
| PFSPZ | LARC2 | Negative after | 81,921 | 11 |
| LARC2_5 | | 6 weeks | | |
| PFSPZ | LARC2 | Negative after | 19,900,587 | 2,689 |
| LARC2_6 | | 6 weeks | | |

¹Each mouse was injected with either wild type (PFSPZ) or LARC2 (PFSPZ LARC2) sporozoites

Blood stage parasites were detected by Giemsa-stained smears in all four mice infected with PFSPZ within 1-3 days of transition to in vitro culture (Table 5). In contrast, no parasites were detected by Giemsa-stained thin smears in all six mice infected with PfSPZ LARC2. On day 7, parasite densities for mice infected with PfSPZ LARC2 ranged between 0-10³ parasite equivalents/ml by P. falciparum 18S qRT-PCR, which suggests that parasite 18S rRNA is released from non-viable and attenuated liver stage parasites of P. falciparum LARC2 into the blood stream. However, viable exo-erythrocytic merozoites are not formed in the P. falciparum LARC2. Livers of infected mice were also harvested on day 7 for extracting RNA and analysis of parasite liver burden by P. falciparum 18S qRT-PCR. Parasite liver load is depicted as log 10 copies of P. falciparum 18S rRNA per μg of total liver RNA (FIG. 10).

### Example 7-Optimize Growth Parameters to be Compatible with GMP Manufacture, and Demonstrate the Production, Purification, and Cryopreservation of Aseptic PfSPZ-LARC2 Vaccine

The production of live aseptic purified and cryopreserved GMP quality PfSPZ-LARC2 Vaccine has been accomplished, and follows the life cycle steps of the genetically attenuated Pf parasite, wherein sporozoites are produced in a biological system—the salivary glands of intact aseptic mosquitoes. This requires simultaneous production of Pf gametocytes from a Master Cell Bank and aseptic mosquitoes from a colony of A. stephensi. Pf were cultured in vitro to produce gametocytes. The P. falciparum infected RBC were mixed with uninfected RBC and Supplemented RPMI. They were then placed into wells of a (6-well) culture plate containing Supplemented RPMI and the culture was expanded. To initiate the production of gametocytes in cultures, complete growth medium (RPMI1640 supplemented with 10% human O+ serum and 367 μM hypoxanthine) was added and changed daily. Giemsa-stained thin blood smears from induced culture samples were checked for monitoring the gametocyte growth. The numbers of stage V gametocytes were assessed on days 14 to 19 after initiation of gametocyte production. Cultures were selected for preparation of an infectious blood meal to feed mosquitoes. The gametocytes in an infectious blood meal were fed to aseptic adult mosquitoes that were cultured from aseptic eggs, larvae and pupae.

Aseptic mosquito production involves all stages of the mosquito life cycle, beginning with disinfection of eggs and preparation of solutions and ending when mosquitoes were ready for feeding (U.S. Pat. No. 8,802,919). An artificial membrane was placed across the mouth of a container and autoclaved. The container was kept around 35-37° C. and adult mosquitoes were introduced. Pf gametocyte-infected blood was added to the membrane and the mosquitoes were allowed to feed for 30 minutes on the gametocyte-infected blood meal. After feeding, the mosquitoes were returned to the incubator and maintained at 26° C., 77% relative humidity, 12-hour light: dark cycles.

The infected aseptic adult mosquitoes were then dissected, the salivary glands harvested, the sporozoites are extracted, purified as described. The aseptic purified PfSPF, vaccine formulated, was then cryopreserved. Cryopreservation begins with the addition of 2×CPA to 1:1 ratio. The preparation was dispensed in aliquots of 20 μL into the cryovials. The vials were prepared for cryopreservation by rate freezing, which is a multistep process that cools the vials to cryogenic temperatures below −150° C. The cryovials were eventually transferred to storage in liquid nitrogen vapor phase (LNVP), which is between −150° C. to −196° C.

The assessment of two separate preparations of PfSPZ-LARC2 Vaccine are shown in Table 6.

TABLE 6
Assessment of clone F7 in mosquito infections with blood meals,
and productivity of parasites in aseptic mosquitoes.

| | 25 Oct. 2021 | | 11 Feb. 2022 (Aseptic) | |
|---|---|---|---|---|
| PfLARC2 clone F7 or NF54 | PfLARC2 | NF54 | PfLARC2 | NF54 |
| Culture Stage V gametocytemia | 1.30 | 1.54 | 1.7 | 2.77 |
| (%)* | | | | |
| Maturation day post induction | 19 | 18 | 19 | 19 |
| Culture hematocrit (%) | 0.76 | 0.96 | 0.84 | 1.09 |
| Final concentration of stage V | 0.08 | 0.19 | 0.10 | 0.12 |
| gametocytes in the blood meal (%) | | | | |
| Exflagellation centers in blood meal | 4.6 | 4.0 | 3.6 | 5.4 |
| (per field) | | | | |
| Pupae Height (cm) | N/A | N/A | 14.4 | 13.3 |
| Volume of infectious bloodmeal | N/A | N/A | 5, 6 | 5, 6 |
| consumed (mL) | | | | |
| Volume of infectious bloodmeal | N/A | N/A | 23.9 | 20.3 |
| consumed (mL)/pupae height (cm) | | | | |
| # Mosq assessed | N/A | N/A | 25 | 25 |
| Prevalence | N/A | N/A | 84% | 64% |
| Mean | N/A | N/A | 109 | 96 |
| Mean of Positives | N/A | N/A | 130 | 150 |
| GeoMean | N/A | N/A | 54 | 22 |
| SPZ/mosquito | 97,811 (80 | 71,502 (40 | 75,497 (~5,000 | 106,189 (~5,000 |
| | mosquitoes | mosquitoes | mosq. Assessed) | mosq. Assessed) |
| | assessed) | assessed) | | |

200 million aseptic, purified PfSPZ-LARC2 were vialed at 10⁶ PfSPZ-LARC2 per vial.

TABLE 7
Oligonucleotide primers

| | | |
|---|---|---|
| | | SEQ ID |
| Primers | Sequence | NO: |

Plasmodium yoelii LINUP knockout

| | | |
|---|---|---|
| PY17X 1465200 | ATAAGCTTGTTTGTATCTTGGATTTTGAAATGGTTG | 5 |
| 5UTR F | | |
| PY17X_1465200 | AATAGCATTGACCAAGCTAGCTATAGGCGCGCCTTTTGGATCAAACGTGGGG | 6 |
| 5UTR R | TATATATG | |
| PY17X_1465200 | TCCAAAAGGCGCGCCTATAGCTAGCTTGGTCAATGCTATTATATATTTTTATT | 7 |
| 3UTR F | ACTTCCC | |
| PY17X_1465200 | TAGCGGCCGCTAACCATTACATATTCGATAATGTAAACAG | 8 |
| 3UTR R | | |
| PY17X_1465200 | TATTCGATGGAGTTGTCTCTGCTT | 9 |
| guide F | | |
| PY17X_1465200 | AAACAAGCAGAGACAACTCCATCG | 10 |
| guide R | | |
| PY17X_1465200 | TTTATAGCTCATTATTCTGGCATACAC | 11 |
| TF | | |
| PY17X_1465200 | GCAATAAAATAAGCAGAATGCGTATGC | 12 |
| TR | | |

Plasmodium yoelii LINUP mCherry

| | | |
|---|---|---|
| PY17X_1465200 | GAATATGTAATGGTTAGGGCCCACAATAATACTCCAACCATCTCTTC | 13 |
| ORF tag F | | |
| PY17X_1465200 | ATACTAGTTTTCTCAAAGTTTAAAATAGTATCTCC | 14 |
| ORF tag R | | |
| PY17X_1465200 | ATCCGCGGTCAATGCTATTATATATTTTTATTACTTCCC | 15 |
| 3UTR tag F | | |
| PY17X_1465200 | GTTGGAGTATTATTGTGGGCCCTAACCATTACATATTCGATAATGTAAACAG | 16 |
| 3UTR tag R | | |
| PY17X_1465200 | AAAAGCTTTAATAAAAGCAGCCAAAGG | 17 |
| Test F | | |
| mCherry Test R | TTCTCCTTCTATTTCAAATTCATGTCC | 18 |
| mCherry Test F | ATGAGAAGAATGATTAAGGATCCCG | 19 |
| PY17X_1465200 | GCAATAAAATAAGCAGAATGCGTATGC | 20 |
| Test R | | |

Plasmodium falciparum LINUP knockout

| | | |
|---|---|---|
| PF3D7_1249700_ | TATTGTTGTTGTTGTTTGCCTTGG | 21 |
| Guide 3F | | |
| PF3D7_1249700_ | AAACCCAAGGCAAACAACAACAAC | 22 |
| Guide 3R | | |
| PF3D7_1249700_ | TATTATATGTGCTACTCCATATAC | 23 |
| Guide 5F | | |
| PF3D7_1249700_ | AAACGTATATGGAGTAGCACATAT | 24 |
| Guide 5R | | |
| PF3D7_1249700_5 | ATACTAGTTTTTATTTTTTATATGAACCATTTAAGAC | 25 |
| UTR F | | |
| PF3D7_1249700_5 | TACTTTTCCCCAACCCGGGTATAGGCGCGCCTCTTTCAACAAAAAATAATAT | 26 |
| UTR R | ATGTGTAC | |
| PF3D7_1249700_3 | TTGTTGAAAGAGGCGCGCCTATACCCGGGTTGGGGAAAAGTAAAATTCGCG | 27 |
| UTR F | ATAAAAATC | |
| PF3D7_1249700_3 | TAGCGGCCGCAATATATATGTGATTTTATTAAGTACGG | 28 |
| UTR R | | |
| PF3D7_1249700_ | TAAATGAGGGATAAAACCTCAAAAAG | 29 |
| Test F | | |
| PF3D7_1249700_ | AATATGTTTCAAACGGTTAAATGTAAG | 30 |
| Test R | | |
| PF3D7_1249700_ | CTTAACAACCATCTTCTTGACAATAGGG | 31 |
| ORF F | | |
| PF3D7_1249700_ | GTTATGTGATTAACTTTCGCTTGATAC | 32 |
| ORF R | | |

TABLE 8
Plasmodium yoelii gene
Plasmodium yoelii gene SEQ ID NO: 33

atgaatatga atttgaaaaa tataaactta gaagacgaaa
aaataaataa aagagtgagt aataaaagaa tagtgcatat
aaaaaataca tatatatctc catatgttct ttataataaa
aaggggaata gtaataatca attggataaa ttaaataaaa
tatttactag tgaaaataat tgtagatttt caaaagaata
ttataattat gttaaagatg gtgatatatg taatagtgga
gaaaaatatc ctgaatgttg ttccccaaat gttaagcaaa
acaatagccc aaattatgac caatgtaata atgaaaaaaa
aatattaaaa tatggcttgt tgaatgaaca aaatatgaaa
tttttaaaaa aaaaaaaaga agaagatttt gaattaaata
caatatctac tatgatacat atttattctg attatgaaag
tgataaagac aatgaaacga taacaaatga atataatgat
ttaatagatt gtataaaaaa aataaatatt tctaataata
aatgtgttat taataatgat aaaaaaatag aggatacttt
attaagtcct tatgaacagt atagaaaatg tagttatcag
acaattgggc ataaagaaaa ttatagtaac aaaaataatg
aaaaaaatag tgacaaaaat aatgaaaaaa atagtgacaa
aaataatgaa aaaaatagtg acaacaatag cgataataat
aatgataaca atagcgacaa taataataac agaaatagcg
ataataataa tagtgatgat aatggagaaa aatgtgtgca
aagattaaat aattttatat ctattccttc agtatcaaac
aaaagttgca tagaacatgt acatgttcag aatgatcata
tggatactgt gtattatgat aattttattt gtaataaaga
aaatcgtgat aataatgaaa atgatactat atataaatgt
gaagaatcta taccacttgg aacaatatta aatattcata
atttagatag taataataat aatggtctaa caacagttat
gttaagaaat ataccaaata aatatacaca aaatatgtta
atggatgtta tgaatgagca ttttaaaggt ttatatgatt
ttttttattt accaattgat tttcgtaaca aatgtaatgt
tggttatgca tttattaatt ttatacatcc tcattatgct
gaactattta ttaaattttt taataattat aaattaaatg
catttaaaag taataaaata tgcactgtta catggggaag
ggtacaagga ttaaaagcta atattgaaca ttacagaaat
tctgcaatta tgactatatc tgtgcctcaa tataagccta
tactttttca aaatggaata tctgtttcat ggcctgaatc
ggatggtccc ttaccagcta taaaacttcg atcacataaa
tattag

TABLE 9
Plasmodium falciparum PlasMei2 gene
Plasmodium falciparum PlasMei2 gene SEQ ID NO: 34
atgcataaga tagatgaaaa gataaatatg aataatgtaa
aaggcactag aattattcatgtaaaaaata catatatatc
tccatacata ctacacaata taaacaataa tgaaggccca
ttaaaaaata tagacaaaat attaaaagaa gaaaaagata
atataaattt aaagatcaataatgttaata ataaggaaac
taataatgat aagcacacat taaaatattc aacattatgt
aatgatctaa atataatgca tacacaaaat aaagaagaag
aaattgaatt aaatacaatttcaacggtta tacacatgaa
ttctgatgaa gatagtgata aagataatga taccataata
aatgaaaaca acgatttgtt aattgataat ataaagaaat
atcatgccgt taaagataatcataataata ttatttataa
tgataaatat aatacaatta ataataattc agtcattaat
gatgtatgta attctattca ttttaataat aattcatata
taactaactt taatttaaatcataattttt ctgtgtgtaa
tcgtacttta aataacacct gcacaagtca ggtaaatttg
agaaataata tgaacagcaa taaaaaaact aatgataaca
ataaaaatct gaacaatgaaattaataaga aaatcaataa
tgataatatt ataaacgaat ttgataatat taataataag
aaaaataatt atattgattg ttccgtttac aaatgtgagg
atgaaatacc ccttggtaccatattaaata tccaagattt
ggatatacat aatagaaata atatgaacaa ttgtaataat
aatattaata ataagagtaa gattcttaca acagttatgc
ttagaaatat tccaaacaaatatacacaaa atatgttgat
ggatgttatg aatgaacatt ttaaaggttt atatgatttt
ttttatttac caattgattt tagaaataaa tgtaatgttg
gatatgcttt tattaatttcatacatccat attatgctga
attgtttatc aaatttttta ataattataa actcaatgca
tttaaaagta acaaagtctg ttctgttact tggggaagag
tacaaggatt aaaggcaaatattgagcatt atagaaattc
agcgattatg accataccaa taccacaata taaacctata
ctttttcaaa atggtataac tgtttcgtgg cctgaatcag
atgggccttt accatccatcaaattaagat ctcaaaaatt
ttaa

TABLE 10
Plasmodium falciparum LINUP gene
Plasmodium falciparum LINUP gene SEQ ID NO: 35

ATGAAAAATGAAGATTCTACATATAAAGAAGATAAGTATT
CAAAATATTCAAAAGAGAATATCCAAAAAATATGTGCTAC
TCCATATACCGGATCTAATATAAGTAACACAGTAGAATCT
GTTTATTTTGTTAATTATAGAGCTGTCGTAGTAAAAACAA
AACATTATTATGTTACTCAAAACTCTTATATGCTTTTTGG
TGATGTCTTTAAAATATATAATTTAAAAGGAGAGATAAAA
AAAATACATGCATTTTTTCCAGGAGATGAAAATAATTTAT
CGGATACATTAATAATGTTAAAAACAACAATAGTTGCTGT
TACTACATCTAAAGGAGAAGCTTTGCGTTTGGCTGATGAA
ATTAATATGACATGTAAAATATGTCATAAGAAAAGAAAAG
CAACCAAATTTTCTATAGAAGGATTTCTAAGAAAAGTTAG
ATGTGACACCTGTAGGGTTAAACCACGAAGTGTAATTTGT
AAAAATATAAATAACAATAAAAAAACAAACAATTCAAAAA
ATAACACAAATAGTAATAATAGTAATAATAATAATAATAA
TAATATAAATAATAATAATAATAATAATAATAATAGTAAT
AATAGTAATAATAAAAATGATTCATTAAAACCAGATGAAT
GTAATATACAGAGTAATCAAAATAAACATCTTACAAAAAA
TAAAATTATTAAATATGAAAATATAGATGACGAAAATATA
AAAAATGAAAAATTTAAAAATATGAATATCAAAAAAGTTA
ATACAAAAAATTGTAATATCACAAATAGTAAGGCAAAAAA
AACTGATAATAATATACAAAATGATATGAATAATAATCAT
CCTAACAACAATTTTGTTAATAATAACATTCTTGACAATA
ATCATATTAATAATAACATTCTTGACAATAATCATATTAA
TAATAACATTCTTGACAATAATCATATTAATAATAATCAT
CTTAATAATAATCATCTTAATAATAATCATCTTAATAATA
ATCATCTTAATAATAATCATCTTAATAATAACCATTTTAA
TAATAATCATCTTAATAATAATCAACTTAACAACCATCTT
CTTGACAATAGGGATAAAATACAATCTCTTCATTACAAAC
ATGCAAATTCTAACTCAGTCCTTAAATATAATAATAGTAA
TAATAATTACTCAGCTAGCCAAAATCAAATTTTTTTTTCA
GAGGAAGAAAATAGAACTCATTTTCTAGAGAATGATCCCA
ATAAGAGGGATGAAATAATTTCGATGATAATTGAACTTAT
AAAATATATCTCATACATGCAAAAGGCATTAATAAAAGCA
TCTGAAGGAAATTTTTTGATTGAAAATCCAGACATCAATG
TAGAGAATGTATCAAAATTGGTTCATTCAGCTCAACAATT
ATGTAACAAATTACTTAACCAAAATAATATAGACGGAGAA
TATGGGAATAATAATTTGTGTGCACCTACTTCGTTGCTTC
TTCACAATAATGAAAAGGGAAATACAATAAAAAATGATGT
TAATATAAATAATATAAGTAATAATAATAATAATAATAAT
TATAATTATAATAATAATAATTATAATAATAATAATATTA
ATTATAATAACTTTAGTAATAATAATCAACTAAAAATTAA
ATACACAAAAATGTATCAAGCGAAAGTTAATCACATAACC
AGAACAAGCAATTTAAATAATAATAATAATAATAATAATA
ATAATACAAATAATACGGATATGTTTTATAATATGAACAA
TCCAAATGATATGAGCACTACAAATACTCACATGACTAAC
CTTTCTTATATTAATAGAAATGACCAAAATAAAAATAATA
ATATGAATAATGATACGGATAGCAATATATATATAAATAA
TAAAAGCAATGATAATAATATTATTAATAATATAAATAAT
ATGAACATAATGAATAATATAAACAATATAAATAATATAA
AAAATATAAATAATATAAATAATATAAAAAATATAAATAA
TATAAATAATATTAATAGTATTAATAGTATTAATAATATG
AATCATATGAATCATATTAATGGTAACAATAATATTAATA
GTAACAATAATATTAATAGTAACAATAATATTAATAGTAA
CAATAATATTAATAGTAACAATAATATTAATAGTAACAAT
AATATTAATAGTAACAATAATATACTTAATAATACAAATG
AAAGAAATAAATATAATATTGAGAACGAATATTCAAAAAA
TATTCAAAATATTCAAAATATACAAAATGTACAAAATAAT
CAATCTTATGTAATACCATATTATTATAATAATCGTCATA
ATATTAACTCTTCGACATCTTTGGATACTTCAACAAATGA
TCAAAAAAATAAATCATATATAAAGGATAATAAAGAATAT
ATGTATGAAAATAATAAGAAGGATGAACACGAAGATAAAA
ACCAATATCATGACCACCAAGGCAAACAACAACAACAACA
ACAACAACAACAACAACAACAACAACAACAACAACAACAA
CAACATCATAATAATAATAATATAAGTCATACAAAAAATA
ATTATTACAATAATAAACAGCAGTTCTTATTTTCTACTTT
ATCAAAACAATATAATACTAATGTCAATATTTCATTTGCT
AATATAAACAATTCTTTATCAAATATTTGTAAGAATGTCT
CTAAAAATTATAATCTCAAAGATACAGTTTTAAATATGGA
AAAGTAA

TABLE 11
Plasmodium falciparum PlasMei2 amino acid
Plasmodium falciparum PlasMei2 amino acid
SEQ ID NO: 36

MHKIDEKINMNNVKGTRIIHVKNTYISPYILHNINNNEGP
LKNIDKILKEEKDNINLKINNVNNKETNNDKHTLKYSTLC
NDLNIMHTQNKEEEIELNTISTVIHMNSDEDSDKDNDTII
NENNDLLIDNIKKYHAVKDNHNNIIYNDKYNTINNNSVIN
DVCNSIHFNNNSYITNFNLNHNFSVCNRTLNNTCTSQVNL
RNNMNSNKKINDNNKNLNNEINKKINNDNIINEFDNINNK
KNNYIDCSVYKCEDEIPLGTILNIQDLDIHNRNNMNNCNN
NINNKSKILTTVMLRNIPNKYTQNMLMDVMNEHFKGLYDF
FYLPIDFRNKCNVGYAFINFIHPYYAELFIKFFNNYKLNA
FKSNKVCSVTWGRVQGLKANIEHYRNSAIMTIPIPQYKPI
LFQNGITVSWPESDGPLPSIKLRSQKF

While illustrative embodiments have been illustrated and described, it will be appreciated that various changes can be made therein without departing from the spirit and scope of the invention.

### REFERENCES

1. World Health O. World malaria report 2020:20 years of global progress and challenges. Geneva: World Health Organization2020.
2 Nussenzweig R S, Vanderberg J, Most H, and Orton C. Protective immunity produced by the injection of x-irradiated sporozoites of Plasmodium berghei. Nature 216:160-162, 1967.
3. Clyde D F. Immunity to falciparum and vivax malaria induced by irradiated sporozoites: a review of the University of Maryland studies, 1971-75. Bull World Health Organ 68 (Suppl): 9-12, 1990.
4. Epstein J E, Tewari K, Lyke K E, Sim B K, Billingsley P F, Laurens M B, Gunasekera A, Chakravarty S, James E R, Sedegah M, Richman A, Velmurugan S, Reyes S, Li M, Tucker K, Ahumada A, Ruben A J, Li T, Stafford R, Eappen A G, Tamminga C, Bennett J W, Ockenhouse C F, Murphy J R, Komisar J, Thomas N, Loyevsky M, Birkett A, Plowe C V, Loucq C, Edelman R, Richie T L, Seder R A, and Hoffman S L. Live attenuated malaria vaccine designed to protect through hepatic CD8+ T cell immunity. Science 334:475-480, 2011.
5. Seder R A, Chang L J, Enama M E, Zephir K L, Sarwar U N, Gordon I J, Holman L A, James E R, Billingsley P F, Gunasekera A, Richman A, Chakravarty S, Manoj A. Velmurugan S. Li M. Ruben A J. Li T. Eappen A G. Stafford R E. Plummer S H. Hendel C S. Novik L. Costner P J. Mendoza F H. Saunders J G. Nason M C. Richardson J H. Murphy J. Davidson S A. Richie T L. Sedegah M. Sutamihardja A. Fahle G A. Lyke K E. Laurens M B. Roederer M. Tewari K. Epstein J E. Sim B K. Ledgerwood J E. Graham B S. Hoffman S L. and for the VRC 312 Study Team. Protection against malaria by intravenous immunization with a nonreplicating sporozoite vaccine. Science 341:1359-1365, 2013.
6. Ishizuka A S. Lyke K E. DeZure A. Berry A A. Richie T L. Mendoza F H. Enama M E. Gordon I J. Chang L J. Sarwar U N. Zephir K L. Holman L A. James E R. Billingsley P F. Gunasekera A. Chakravarty S. Manoj A. Li M. Ruben A J. Li T. Eappen A G. Stafford R E. K C N. Murshedkar T. DeCederfelt H. Plummer S H. Hendel C S. Novik L. Costner P J. Saunders J G. Laurens M B. Plowe C V. Flynn B. Whalen W R. Todd J P. Noor J. Rao S. Sierra-Davidson K. Lynn G M. Epstein J E. Kemp M A. Fahle G A. Mikolajczak S A. Fishbaugher M. Sack B K. Kappe S H. Davidson S A. Garver L S. Bjorkstrom N K. Nason M C. Graham B S. Roederer M. Sim B K. Hoffman S L. Ledgerwood J E, and Seder R A. Protection against malaria at 1 year and immune correlates following PfSPZ vaccination. Nat Med 22:614-623, 2016.
7. Epstein J E. Paolino K M. Richie T L. Sedegah M. Singer A. Ruben A J. Chakravarty S. Stafford A. Ruck R C. Eappen A G. Li T. Billingsley P F. Manoj A. Silva J C. Moser K. Nielsen R. Tosh D. Cicatelli S. Ganeshan H. Case J. Padilla D. Davidson S. Garver L. Saverino E. Murshedkar T. Gunasekera A. Twomey P S. Reyes S. Moon J E. James E R. Kc N. Li M. Abot E. Belmonte A. Hauns K. Belmonte M. Huang J. Vasquez. C. Remich S. Carrington M. Abebe Y. Tillman A. Hickey B. Regules J. Villasante E. Sim B K. and Hoffman S L. Protection against Plasmodium falciparum malaria by PfSPZ Vaccine. JCI Insight 2: e89154, 2017.
8. Lyke K E. Ishizuka A S. Berry A A. Chakravarty S. DeZure A. Enama M E. James E R. Billingsley P F. Gunasekera A. Manoj A. Li M. Ruben A J. Li T. Eappen A G. Stafford R E. Kc N. Murshedkar T. Mendoza F H. Gordon I J. Zephir K L. Holman L A. Plummer S H. Hendel C S. Novik L. Costner P J. Saunders J G. Berkowitz. N M. Flynn B J. Nason M C. Garver L S. Laurens M B. Plowe C V. Richie T L. Graham B S. Roederer M. Sim B K. Ledgerwood J E. Hoffman S L. and Seder R A. Attenuated PfSPZ Vaccine induces strain-transcending T cells and durable protection against heterologous controlled human malaria infection. Proc Natl Acad Sci USA 114:2711-2716, 2017.
9. Sissoko M S. Healy S A. Katile A. Omaswa F. Zaidi I. Gabriel E E. Kamate B. Samake Y. Guindo M A. Dolo A. Niangaly A. Niare K. Zeguime A. Sissoko K. Diallo H. Thera I. Ding K. Fay M P. O'Connell E M. Nutman T B. Wong-Madden S. Murshedkar T. Ruben A J. Li M. Abebe Y. Manoj A. Gunasekera A. Chakravarty S. Sim B K. Billingsley P F. James E R. Walther M. Richie T L. Hoffman S L. Doumbo O, and Duffy P E. Safety and efficacy of PfSPZ Vaccine against Plasmodium falciparum via direct venous inoculation in healthy malaria-exposed adults in Mali: a randomised, double-blind phase 1 trial. Lancet Infect Dis 17:498-509, 2017.
10. Jongo S A. Shekalage S A. Church L W P. Ruben A J. Schindler T. Zenklusen I. Rutishauser T. Rothen J. Tumbo A. Mkindi C. Mpina M. Mtoro A T. Ishizuka A S. Ramadhani K K. Milando F A. Qassim M. Juma O A. Mwakasungula S. Simon B. James E R. Abebe Y. K C N. Chakravarty S. Saverino E. Bakari B M. Billingsley P F. Seder R A. Daubenberger C. Sim B K L. Richie T L. Tanner M. Abdulla S. and Hoffman S L. Safety. Immunogenicity, and Protective Efficacy against Controlled Human Malaria Infection of Plasmodium falciparum Sporozoite Vaccine in Tanzanian Adults. Am J Trop Med Hyg 99:338-349, 2018.
11. Olotu A. Urbano V. Hamad A. Eka M. Chemba M. Nyakarungu E. Raso J. Eburi E. Mandumbi D O. Hergott D. Maas C D. Ayekaba M O. Milang D N. Rivas M R. Schindler T. Embon O M. Ruben A J. Saverino E. Abebe Y. Kc N. James E R. Murshedkar T. Manoj A. Chakravarty S. Li M. Adams M. Schwabe C. Segura L. Daubenberger C. Tanner M. Richie T L. Billingsley P F. Sim B K L. Abdulla S. and Hoffman S L. Advancing Global Health through Development and Clinical Trials Partnerships: A Randomized. Placebo-Controlled. Double-Blind Assessment of Safety. Tolerability, and Immunogenicity of Plasmodium falciparum Sporozoites Vaccine for Malaria in Healthy Equatoguinean Men. Am J Trop Med Hyg 98:308-318, 2018.
12. Jongo S A. Church L W P. Mtoro A T. Chakravarty S. Ruben A J. Swanson P A. Kassim K R. Mpina M. Tumbo A M. Milando F A. Qassim M. Juma O A. Bakari B M. Simon B. James E R. Abebe Y. Kc N. Saverino E. Gondwe L. Studer F. Fink M. Cosi G. El-Khorazaty J. Styers D. Seder R A. Schindler T. Billingsley P F. Daubenberger C. Sim B K L. Tanner M. Richie T L. Abdulla S. and Hoffman S L. Safety and Differential Antibody and T-Cell Responses to the Plasmodium falciparum Sporozoite Malaria Vaccine. PfSPZ Vaccine, by Age in Tanzanian Adults. Adolescents. Children, and Infants. Am J Trop Med Hyg 100:1433-1444, 2019.
13. Sissoko M S. Healy S A. Katile A. Zaidi I. Hu Z. Kamate B. Samake Y. Sissoko K. Mwakingwe-Omari A. Lane J. Imeru A. Mohan R. Thera I. Guindo C O. Dolo A. Niare K. Koïta F. Niangaly A. Rausch K M. Zeguime A. Guindo M A. Bah A. Abebe Y. James E R. Manoj A. Murshedkar T. Kc N. Sim B K L. Billingsley P F. Richie T L. Hoffman S L. Doumbo O. and Duffy P E. Safety and efficacy of a three-dose regimen of Plasmodium falciparum sporozoite vaccine in adults during an intense malaria transmission season in Mali: a randomised, controlled phase 1 trial. Lancet Infect Dis 22:377-389, 2022.
14. Chattopadhyay R. Conteh S. Li M. James E R. Epstein J E. and Hoffman S L. The Effects of radiation on the safety and protective efficacy of an attenuated Plasmodium yoelii sporozoite malaria vaccine. Vaccine 27:3675-3680, 2009.
15. Hoffman S L. Billingsley P. James E. Richman A. Loyersky M. Li T. 15. Charkravarty S. Gunasekera A. Chattopadhyay R. Li M. Stafford R. Ahumada A. Epstein J E. Sedegah M. Reyes S. Richie T L. Lyke K E. Edelman R. Laurens M B. Plowe C V. and Sim B K L. Development of a metabolically active, non-replicating sporozoite vaccine to prevent Plasmodium falciparum malaria. Human Vaccines 6:97-106, 2010.
16. Hoffman S L. Goh L M. Luke T C. Schneider I. Le T P. Doolan D L. Sacci J. de la Vega P. Dowler M. Paul C. Gordon D M. Stoute J A. Church L W. Sedegah M. Heppner D G. Ballou W R, and Richie T L. Protection of humans against malaria by immunization with radiation-attenuated Plasmodium falciparum sporozoites. J Infect Dis 185:1155-1164, 2002.
17. Richie T L. Billingsley P F. Sim B K. Epstein J E. Lyke K E, Mordmuller B. Alonso P. Duffy P E. Doumbo O K. Sauerwein R W. Tanner M. Abdulla S. Kremsner P G. Seder R A. and Hoffman S L. Progress with Plasmodium falciparum sporozoite (PfSPZ)-based malaria vaccines. Vaccine 33:7452-7461, 2015.
18. Mwakingwe-Omari A. Healy S A. Lane J. Cook D M. Kalhori S. Wyatt C. Kolluri A. Marte-Salcedo O. Imeru A. Nason M. Ding L K. Decederfelt H. Duan J. Neal J. Raiten J. Lee G. Hume J C C. Jeon J E. Ikpeama I. Kc N. Chakravarty S. Murshedkar T. Church L W P. Manoj A. Gunasekera A. Anderson C. Murphy S C. March S. Bhatia S N. James E R. Billingsley P F. Sim B K L. Richie T L. Zaidi I. Hoffman S L. and Duffy P E. Two chemoattenuated PfSPZ malaria vaccines induce sterile hepatic immunity. Nature 595:289-294, 2021.
19. Aly A S. Mikolajczak S A. Rivera H S. Camargo N. Jacobs-Lorena V. Labaied M. Coppens I. and Kappe S H. Targeted deletion of SAPI abolishes the expression of infectivity factors necessary for successful malaria parasite liver infection. Mol Microbiol 69:152-163, 2008.
20. Labaied M. Harupa A. Dumpit R F. Coppens I. Mikolajczak S A. and Kappe S H. Plasmodium yoelii sporozoites with simultaneous deletion of P52 and P36 are completely attenuated and confer sterile immunity against infection. Infect Immun 75:3758-3768, 2007.
21. Mueller A K. Camargo N. Kaiser K. Andorfer C. Frevert U. Matuschewski K. and Kappe S H. Plasmodium liver stage developmental arrest by depletion of a protein at the parasite-host interface. Proc Natl Acad Sci USA 102:3022-3027, 2005.
22. Mueller A K. Labaied M. Kappe S H. and Matuschewski K. Genetically modified Plasmodium parasites as a protective experimental malaria vaccine. Nature 433:164-167, 2005.
23. Roestenberg M. Walk J. van der Boor S C. Langenberg M C C. Hoogerwerf M A. Janse J J. Manurung M. Yap X Z. Garcia A F. Koopman J P R. Meij P. Wessels E. Teelen K. van Waardenburg Y M. van de Vegte-Bolmer M. van Gemert G J. Visser L G. van der Ven A. de Mast Q. Natasha K C. Abebe Y. Murshedkar T. Billingsley P F. Richie T L. Sim B K L. Janse C J. Hoffman S L. Khan S M. and Sauerwein R W. A double-blind, placebo-controlled phase 1/2a trial of the genetically attenuated malaria vaccine PfSPZ-GA1. Sci Transl Med 12: eaaz5629, 2020.
24. Dankwa D A. Davis M J. Kappe S H I, and Vaughan A M. A Plasmodium yoelii Mei2-Like RNA Binding Protein Is Essential for Completion of Liver Stage Schizogony. Infect Immun 84:1336-1345, 2016.
25. Goswami D. Betz. W. Locham N K. Parthiban C. Brager C. Schäfer C. Camargo N. Nguyen T. Kennedy S Y. Murphy S C. Vaughan A M. and Kappe S H I. A replication-competent late liver stage-attenuated human malaria parasite. JCI Insight 5: e135589, 2020.
26. Vaughan A M. Sack B K. Dankwa D. Minkah N. Nguyen T. Cardamone H. and Kappe S H I. A Plasmodium Parasite with Complete Late Liver Stage Arrest Protects against Preerythrocytic and Erythrocytic Stage Infection in Mice. Infect Immun 86: e00088-18, 2018.
27. Vaughan A M. Kappe S H. and Dankwa D A. Doubly attenuated late liver stage malaria parasites and related compositions and methods. U.S. Pat. No. 10,905,753, issued Feb. 2, 2021.
28. Butler N S. Schmidt N W. Vaughan A M. Aly A S. Kappe S H, and Harty J T. Superior antimalarial immunity after vaccination with late liver stage-arresting genetically attenuated parasites. Cell Host Microbe 9:451-462, 2011.
29. Sack B K. Keitany G J. Vaughan A M. Miller J L. Wang R. and Kappe S H. Mechanisms of stage-transcending protection following immunization of mice with late liver stage-arresting genetically attenuated malaria parasites. PLOS Pathog 11: e1004855, 2015.
30. van Schaijk B C. Ploemen I H. Annoura T. Vos M W. Foquet L, van Gemert G J. Chevalley-Maurel S, van de Vegte-Bolmer M. Sajid M. Franetich J F. Lorthiois A. Leroux-Roels G. Meuleman P. Hermsen C C. Mazier D. Hoffman S L. Janse C J. Khan S M. and Sauerwein R W. A genetically attenuated malaria vaccine candidate based on P. falciparum b9/slarp gene-deficient sporozoites. Elife: 3, 2014.
31. Gomez-Perez G P. Legarda A. Munoz. J. Sim B K. Ballester M R. Dobano C. Moncunill G. Campo J J. Cistero P. Jimenez. A. Barrios D. Mordmuller B. Pardos J. Navarro M. Zita C J. Nhamuave C A. Garcia-Basteiro A L. Sanz. A. Aldea M. Manoj A. Gunasekera A. Billingsley P F. Aponte J J. James E R. Guinovart C. Antonijoan R M. Kremsner P G. Hoffman S L. and Alonso P L. Controlled human malaria infection by intramuscular and direct venous inoculation of cryopreserved Plasmodium falciparum sporozoites in malaria-naive volunteers: effect of injection volume and dose on infectivity rates. Malar J 14:306, 2015.
32. Mordmüller B. Supan C. Sim K L. Gómez-Perez G P. Ospina Salazar C L. Held J. Bolte S. Esen M. Tschan S. Joanny F. Lamsfus Calle C. Löhr S J Z. Lalremruata A. Gunasekera A. James E R. Billingsley P F. Richman A. Chakravarty S. Legarda A. Muñoz. J. Antonijoan R M. Ballester M R. Hoffman S L. Alonso P L. and Kremsner P G. Direct venous inoculation of Plasmodium falciparum sporozoites for controlled human malaria infection: a dose-finding trial in two centres. Malar J 14:117, 2015.
33. Eappen A G. and Hoffman S L. Infectious Plasmodium sporozoites grown in vitro. U.S. Pat. No. 9,878,026, issued Jan. 30, 2018.
34. Eappen A G. and Hoffman S L. “Infectious Plasmodium sporozoites grown in vitro” U.S. Pat. No. 10,441,646 B2, issued Oct. 15, 2019
35. Eappen A G. and Hoffman S L. Infectious plasmodium sporozoites grown in vitro. U.S. Pat. No. 11,207,395, issued Dec. 28, 2021.
36. Sim B K L. Li M. Stafford R E. and Hoffman S L. Purified plasmodium and vaccine compositions. U.S. Pat. No. 8,043,625, issued Oct. 25, 2011, 2011.
37. Sim B K L. Li M. Stafford R E. and Hoffman S L. Purified plasmodium and vaccine compositions. U.S. Pat. No. 9,241,982, issued Jan. 26, 2016.
38. Hoffman S L. and Luke T C. Apparatuses and methods for the production of haematophagous organisms and parasites suitable for vaccine production. U.S. Pat. No. 8,802,919, issued Aug. 12, 2014.
39. Chakravarty S. Hoffman S L, and Tsuji M. Pharmaceutical compositions comprising attenuated Plasmodium sporozoites and glycolipid adjuvants. U.S. Pat. No. 9,278,125, issuend Mar. 8, 2016.
40). Provinciali M. Di Stefano G. and Fabris N. Optimization of cytotoxic assay by target cell retention of the fluorescent dye carboxyfluorescein diacetate (CFDA) and comparison with conventional 51C R release assay. J Immunol Methods 155:19-24, 1992.
41. Vollenweider I. and Groscurth P. Comparison of four DNA staining fluorescence dyes for measuring cell proliferation of lymphokine-activated killer (LAK) cells. J Immunol Methods 149:133-135, 1992.
42. Loeffler D A. Kukuruga M A. Juneau P L. and Heppner G H. Analysis of distribution of tumor- and preneoplasia-infiltrating lymphocytes using simultaneous Hoechst 33342 labeling and immunophenotyping. Cytometry 13:169-174, 1992.
43. Rivoltini L. Arienti F. Orazi A. Cefalo G. Gasparini M. Gambacorti-Passerini C. Fossati-Bellani F. and Parmiani G. Phenotypic and functional analysis of lymphocytes infiltrating paediatric tumours, with a characterization of the tumour phenotype. Cancer Immunol Immunother 34:241-251, 1992.
44. Chang A E. Yoshizawa H. Sakai K. Cameron M J. Sondak V K. and Shu S. Clinical observations on adoptive immunotherapy with vaccine-primed T-lymphocytes secondarily sensitized to tumor in vitro. Cancer Res 53:1043-1050, 1993.
45. Coligan J E. Kruisbeek A M. Margulies D H. Shevach E M. and Strober W. Current Protocols in Immunology. New York: John Wiley & Sons 2000.
46. Adejare A. Remington, the science and practice of pharmacy. 2020.
47. Ishino T. Boisson B. Orito Y. Lacroix C. Bischoff E. Loussert C. Janse C. Menard R. Yuda M. and Baldacci P. LISP1 is important for the egress of Plasmodium berghei parasites from liver cells. Cell Microbiol 11:1329-1339, 2009.
48. Orito Y. Ishino T. Iwanaga S. Kaneko I. Kato T. Menard R. Chinzei Y. and Yuda M. Liver-specific protein 2: a Plasmodium protein exported to the hepatocyte cytoplasm and required for merozoite formation. Mol Microbiol 87:66-79, 2013.
49. Kumar H. Sattler J M. Singer M. Heiss K. Reinig M. Hammerschmidt-Kamper C. Heussler V. Mueller A K. and Frischknecht F. Protective efficacy and safety of liver stage attenuated malaria parasites. Sci Rep 6:26824, 2016.
50. Gupta D K. Dembele L. Voorberg-van der Wel A. Roma G. Yip A. Chuenchob V. Kangwanrangsan N. Ishino T. Vaughan A M. Kappe S H. Flannery E L. Sattabongkot J. Mikolajczak S. Bifani P. Kocken C H, and Diagana T T. The Plasmodium liver-specific protein 2 (LISP2) is an early marker of liver stage development. Elife 82019.
51. Haussig J M. Matuschewski K. and Kooij T W. Inactivation of a Plasmodium apicoplast protein attenuates formation of liver merozoites. Mol Microbiol 81:1511-1525, 2011.
52. Siau A. Silvie O. Franetich J F. Yalaoui S. Marinach C. Hannoun L, van Gemert G J. Luty A J. Bischoff E. David P H. Snounou G. Vaquero C. Froissard P. and Mazier D. Temperature shift and host cell contact up-regulate sporozoite expression of Plasmodium falciparum genes involved in hepatocyte infection. PLOS Pathog 4: e1000121, 2008.
53. Pei Y. Tarun A S. Vaughan A M. Herman R W. Soliman J M. Erickson-Wayman A. and Kappe S H. Plasmodium pyruvate dehydrogenase activity is only essential for the parasite's progression from liver infection to blood infection. Mol Microbiol 75:957-971, 2010.
54. Vaughan A M. O'Neill M T. Tarun A S. Camargo N. Phuong T M. Aly A S. Cowman A F. and Kappe S H. Type I I fatty acid synthesis is essential only for malaria parasite late liver stage development. Cell Microbiol 11:506-520, 2009.
55. Yu M. Kumar T R. Nkrumah L J. Coppi A. Retzlaff S. Li C D. Kelly B J. Moura 40) 55. P A. Lakshmanan V. Freundlich J S. Valderramos J C. Vilcheze C. Siedner M. Tsai J H. Falkard B. Sidhu A B. Purcell L A. Gratraud P. Kremer L. Waters A P. Schiehser G. Jacobus D P. Janse C J. Ager A. Jacobs W R. Jr., Sacchettini J C. Heussler V. Sinnis P. and Fidock D A. The fatty acid biosynthesis enzyme Fabl plays a key role in the development of liver-stage malarial parasites. Cell Host Microbe 4:567-578, 2008.

1. A live Plasmodium-species organism that is genetically modified to disrupt a LINUP gene that encodes a Liver Stage Nuclear Protein, thereby preventing the biological function of a protein encoded by the wildtype LINUP gene.

2. The live Plasmodium-species organism of claim 1 that arrests life cycle development in late liver stage within a mammalian intermediate host.

3. The Plasmodium-species organism of claim 2, wherein the Plasmodium-species comprises P. falciparum, P. vivax, P. malariae, P. ovale, or P. knowlesi.

4. The Plasmodium-species organism of claim 3 wherein the Plasmodium-species comprises P. falciparum.

5. The Plasmodium-species organism of claim 3, wherein the organism is at the sporozoite stage of development. 6 The Plasmodium-species organism of claim 3 wherein the functional LINUP gene prior to genetic modification is a LINUP that has at least about 40%, 50%, 60%, 70%, 80%, 90%, or 95% sequence identity to SEQ ID NO: 35.

7. The Plasmodium-species organism of claim 3, wherein a functional LINUP gene prior to genetic modification encodes a LINUP polypeptide with at least about 40%, 50%, 60%, 70%, 80%, 90%, or 95% sequence identity to the amino acid sequence set forth in PF3D7_1249700, SEQ ID NO: 1.

8. A vaccine composition comprising the Plasmodium-species organism of claim 5 and an additive, where the vaccine composition is prepared aseptically.

9. The Plasmodium-species organism of claim 2, wherein the Plasmodium organism comprises at least one transgene.

10. The Plasmodium-species organism of claim 9, wherein the transgene is under control of a promoter that results in transcription of the transgene during the sporozoite or liver stage of development.

11. The Plasmodium-species organism of claim 2, wherein the Plasmodium organism is genetically modified to disrupt one or more additional gene functions, each additional gene function necessary for Plasmodium organism life cycle transit through liver stage and to blood stage.

12. The Plasmodium-species organism of claim 11, wherein the Plasmodium organism is genetically modified to disrupt PlasMei2 gene function.

13. The Plasmodium-species organism of claim 12 wherein the Plasmodium organism is at the sporozoite stage of development.

14. The Plasmodium-species organism of claim 12, wherein a functional PlasMei2 gene prior to genetic modification comprises a nucleic acid sequence that is at least at least about 70%, 80%, 90%, or 95% identity to the nucleic acid sequence set forth in SEQ ID NO:34.

15. The Plasmodium-species organism of claim 12, wherein the amino acid sequence of a functional PlasMei2 polypeptide encoded by the PlasMei2 gene comprises is at least at least about 70%, 80%, 90%, or 95% identity to the sequence set forth in SEQ ID NO: 36.

16. A vaccine composition comprising the Plasmodium-species organism of claim 13 and an additive, where the vaccine composition is prepared aseptically.

17. A method for inducing an immune response in a human subject against one or more Plasmodium antigens, the method comprising administering to the subject one or more doses of the vaccine composition of claim 8 or claim 16.

18. The method of claim 17, wherein the immune response ameliorates or protects against infection from a subsequent wildtype Plasmodium challenge.

19. A method of conferring protective immunity in a human subject against malaria caused by a Plasmodium-species parasite, the method comprising administering to the human subject one or more doses of the Plasmodium-species organism recited in claim 8 or claim 16.

20. The Plasmodium-species organism of claim 5 that is produced in vitro.

21. The Plasmodium-species organism of claim 13 that is produced in vitro.


SEQUENCE LISTING (36 sequences)

SEQ ID NO: 1 - protein, 908 aa, Plasmodium falciparum
MKNEDSTYKEDKYSKYSKENIQKICATPYTGSNISNTVESVYFVNYRAVVVKTKHYYVTQNSYMLFGDVFKIYNLKGEIKKIHAFFPGDENNLSDTLIMLKTTIVAVTTSKGEALRLADEINMTCKICHKKRKATKFSIEGFLRKVRCDTCRVKPRSVICKNINNNKKTNNSKNNTNSNNSNNNNNNNINNNNNNNNNSNNSNNKNDSLKPDECNIQSNQNKHLTKNKIIKYENIDDENIKNEKFKNMNIKKVNTKNCNITNSKAKKTDNNIQNDMNNNHPNNNFVNNNILDNNHINNNILDNNHINNNILDNNHINNNHLNNNHLNNNHLNNNHLNNNHLNNNHFNNNHLNNNQLNNHLLDNRDKIQSLHYKHANSNSVLKYNNSNNNYSASQNQIFFSEEENRTHFLENDPNKRDEIISMIIELIKYISYMQKALIKASEGNFLIENPDINVENVSKLVHSAQQLCNKLLNQNNIDGEYGNNNLCAPTSLLLHNNEKGNTIKNDVNINNISNNNNNNNYNYNNNNYNNNNINYNNFSNNNQLKIKYTKMYQAKVNHITRTSNLNNNNNNNNNNTNNTDMFYNMNNPNDMSTTNTHMTNLSYINRNDQNKNNNMNNDTDSNIYINNKSNDNNIINNINNMNIMNNINNINNIKNINNINNIKNINNINNINSINSINNMNHMNHINGNNNINSNNNINSNNNINSNNNINSNNNINSNNNINSNNNILNNTNERNKYNIENEYSKNIQNIQNIQNVQNNQSYVIPYYYNNRHNINSSTSLDTSTNDQKNKSYIKDNKEYMYENNKKDEHEDKNQYHDHQGKQQQQQQQQQQQQQQQQQQQHHNNNNISHTKNNYYNNKQQFLFSTLSKQYNTNVNISFANINNSLSNICKNVSKNYNLKDTVLNMEK

SEQ ID NO: 2 - protein, 746 aa, Plasmodium yoelii
MKKSKAETTPSKNNKCENPVDSKDEKDSISKYPTASVPNELVDYVYFVNYRAVVVKTKHYYATQNSYILFGDVFKIYNLKGEIKKIHAFFPGDQNNLSDTLIMLKTTIIAVTTSKAEALRVADEIEMICKICHKKRKATKFSIEGFLRKVRCDTCRVKPKHFINKNNTNNNTTTTTTNNNNNTKQKNNNYKNEPNHDSIQSKKNCISCSTITENYSNSYNNTNSRLNLNTNKNIPTSNNENRHNVNSSNIQDKINITNYINDGNTITINNTINKEHSCQYNTKFNSNNNNTTNSIVKNFCFNNNIESYNTSALLCDNNINTNDSVSNNVTGSNKSINNPNDYKNKDEIVNMIIELIKYISWMQKALIKAAKGNNYSIDNSEINIDKISQIVYSAQLLCNKLLRQKSLLDISNNNNTPTISSLLPFYNNQNNNITMETNSNPVNTSGTATTSNTATASTATANDVNIKPKYSNLYAQKINTINKQNEINNPNSSNIFTNFINQNNITTSNNTNGITRNPRTCILNPHGNNNNNIEAGDNPSGNNNTSSNNGNTNCSENSNTTTNDNNNNNNINETNDKKSRNNKNINNKIINEIETTDNINNSINPNNHNDINNNINSINNINSNLDIENINNYNNMNCNVIPYHANNCQVNCLTSLQSDRNYTRLTPFIKNENNYLCLNNKNNDFTNLVTSKTNYYNDKQDIVFSTLSKQYNSNISFTNINNTVSNFYKAVAKNYNIGDTILNFEK

SEQ ID NO: 3 - protein, 816 aa, Plasmodium vivax
MKRAKDEQTPSDEDKYEKYSLYKSDKASPGGQIPPPSSAMTSQVESVYFVNYRAVVVKTKRYYVTQNSYMLFGDVFKIYNLKGEIKKIHAFFPGDENNLSDTLIMLKTTVVAVTTSKAEALRVADGIEMTCKICRKKRKATKFSIEGFLRKVRCDTCRVKPKNLINKMRRVNNVKGEQQTFLAVSSNTTNDSSCTPSAAAAMATSVNPSRTGRKSAPLCNDAAGASNALCLSNGAAPGDGPSSGAMSGESIGTASGASGGAPMATPNGATTLGGSNGMGSNSVGNGSLANNSVGSSVGSSVGSSVGSSVGSGVGSGVGNGVGSGVGSGVGNGVGSGVGNGSLANNSAGESARMHSGEANLGGNPSSGGTPHNGCLSASAAAMNDAGTGQHHHQQRICSYNSKNASSGSGAHSFLKSSCYIGAGEACTAGHSSHGSHNNHNNHNNHNNHNEPDGKNPDEIIAMIIELIKYISWMQKALMRASKGNYVIDHTEINIEKISKVVHSAQVLCNKLLRQKSLVDQTGSNSRNFTNFIPMYNTNSNNLRMKYANLYENKMNAIHSRPIETNLANSNAVNVFNNIINHNTLSLSNCAAAQRNVSATPSCSSGGSGGSSGSAGNSGGSGGSSGSAGNSGGSGGNRTGNDSASNSTSGEGAPNESGPNGTSPTNISNLTSVTNLTNAPSNGSNSNATMETDSPLNNFSTINNRNILPYYSNACQITCVPPSPCSTRNDSRMNHFIKQENNYMYRNKQTEEFPNMQSNNKNSYYTDKQDIVFSALSKQYNSNVNISFNNINNSVSNFYRTVSKNYNIRDTILNLDK

SEQ ID NO: 4 - protein, 950 aa, synthetic construct
  VARIANT 1: note = not present or Met
  VARIANT 2: note = not present or Lys
  VARIANT 3: note = not present or Arg
  VARIANT 4: note = Met Ser or Ala
  VARIANT 6: note = Asn, Ala or Asp
  VARIANT 8: note = Asp, Thr or Gln
  VARIANT 9: note = Ser or Thr
  VARIANT 10: note = Thr or Pro
  VARIANT 11: note = Tyr or Ser
  VARIANT 12: note = Lys or Asp
  VARIANT 13: note = Glu or Asn
  VARIANT 16: note = Lys or Cys
  VARIANT 17: note = Ser or Glu
  VARIANT 18: note = Lys or Asn
  VARIANT 19: note = Tyr or Pro; note = Tyr or Pro
  VARIANT 20: note = Ser of Val
  VARIANT 21: note = Lys, Asp or Leu
  VARIANT 22: note = Glu, Ser or Tyr
  VARIANT 23: note = Asn or Lys
  VARIANT 24: note = Ile, Asp or Ser
  VARIANT 25: note = Glun, Glu or Asp
  VARIANT 27: note = Ile, Asp or Ala
  VARIANT 28: note = Cys or Ser
  VARIANT 29: note = Ail, Ile or Pro
  VARIANT 30: note = Thr, Ser or Gly
  VARIANT 31: note = Pro, Lys or Gly
  VARIANT 32: note = Tyr or Gln
  VARIANT 33: note = Not present or Ile
  VARIANT 34: note = Not present or Pro
  VARIANT 35: note = Thr or Pro
  VARIANT 36: note = Gly, Thr or Pro
  VARIANT 37: note = Ser or Ala
  VARIANT 38: note = Asn or Ser
  VARIANT 39: note = Ile, Val or Ala
  VARIANT 40: note = Ser, Pro or Met
  VARIANT 41: note = Asn or Thr
  VARIANT 42: note = Thr, Glu or Ser
  VARIANT 43: note = Not present, Leu or Gln
  VARIANT 45: note = Glu or Asp
  VARIANT 46: note = Ser or Tyr
  VARIANT 61: note = His or Arg
  VARIANT 64: note = Val or Ala
  VARIANT 70: note = Met or Ile
  VARIANT 110: note = Ile or Val
  VARIANT 111: note = Ile or Val
  VARIANT 118: note = Gly or Ala
  VARIANT 123: note = Leu or Val
  VARIANT 126: note = Glu or Gly
  VARIANT 128: note = Asn or Glu
  VARIANT 128: note = Asn or Glu
  VARIANT 130: note = Ile or Thr
  VARIANT 135: note = His or Arg
  VARIANT 162: note = Arg or Lys
  VARIANT 163: note = Ser, His or Asn
  VARIANT 164: note = Val, Phe or Leu
  VARIANT 166: note = Cys or Asn
  VARIANT 168: note = Asn or Met
  VARIANT 169: note = Ile, not present or Arg
  VARIANT 170: note = Asn, not present or Arg
  VARIANT 171: note = Asn, not present or Val
  VARIANT 172: note = Asn or not present
  VARIANT 173: note = Lys, not present or Asn
  VARIANT 174: note = Lys, not present or Val
  VARIANT 175: note = Thr, not present or Lys
  VARIANT 176: note = Asn, not present or Gly
  VARIANT 177: note = Asn or Glu
  VARIANT 178: note = Ser, Thr or Gln
  VARIANT 179: note = Lys, Asn or Gln
  VARIANT 180: note = Asn or Thr
  VARIANT 181: note = Asn or Phe
  VARIANT 182: note = Thr or Leu
  VARIANT 183: note = Asn, Thr or Ala
  VARIANT 184: note = Ser, Thr or Val
  VARIANT 185: note = Asn, Thr or Ser
  VARIANT 186: note = Asn, Thr or Ser
  VARIANT 187: note = Ser, Thr or Asn
  VARIANT 188: note = Asn or Thr
  VARIANT 189: note = Asn or Thr
  VARIANT 192: note = Asn or Ser
  VARIANT 193: note = Asn, Thr or Ser
  VARIANT 194: note = Asn, Lys or Cys
  VARIANT 195: note = Ile, Gln or Thr
  VARIANT 196: note = Asn, Lys or Phe
  VARIANT 197: note = Asn or Ser
  VARIANT 198: note = Asn or Ala
  VARIANT 199: note = Asn or Ala
  VARIANT 200: note = Asn, Tyr or Ala
  VARIANT 201: note = Asn, Lys or Ala
  VARIANT 202: note = Asn or Met
  VARIANT 203: note = Asn, not present or Ala
  VARIANT 204: note = Asn, not present or Thr
  VARIANT 205: note = Ser or not present
  VARIANT 206: note = Asn, not present of Val
  VARIANT 207: note = Asn or not present
  VARIANT 208: note = Ser, not present or Pro
  VARIANT 209: note = Asn, not present or Ser
  VARIANT 210: note = Asn, not present or Arg
  VARIANT 211: note = Lys, not present or Thr
  VARIANT 212: note = Asn, not present or Gly
  VARIANT 213: note = Asp, not present or Arg
  VARIANT 214: note = Ser, not present or Lys
  VARIANT 215: note = Leu, not present or Ser
  VARIANT 216: note = Lys, Glu or Ala
  VARIANT 218: note = Asp, Asn or Leu
  VARIANT 219: note = Glu, His or Cys
  VARIANT 220: note = Cys, Asp or Asn
  VARIANT 221: note = Asn, Ser or Asp
  VARIANT 222: note = Ile or Ala
  VARIANT 223: note = Gln or Ala
  VARIANT 224: note = Ser or Gly
  VARIANT 225: note = Asn, Lys or Ala
  VARIANT 226: note = Gln, Lys or Ser
  VARIANT 228: note = Lys, Cys or Ala
  VARIANT 229: note = His, Ile or Leu
  VARIANT 230: note = Leu, Ser or Cys
  VARIANT 231: note = Thr, Cys or Leu
  VARIANT 232: note = Lys or Ser
  VARIANT 233: note = Asn or Thr
  VARIANT 234: note = Lys, Ile or Gly
  VARIANT 235: note = Ile, Thr or Ala
  VARIANT 236: note = Ile, Glu or Ala
  VARIANT 237: note = Lys, Asn or Pro
  VARIANT 238: note = Tyr or Gly
  VARIANT 239: note = Glu, Ser or Asp
  VARIANT 240: note = Asn or Gly
  VARIANT 241: note = Ile, not present or Pro
  VARIANT 242: note = Asp, not present or Ser
  VARIANT 243: note = Asp, not present or Ser
  VARIANT 244: note = Glu, not present or Gly
  VARIANT 245: note = Asn, not present or Ala
  VARIANT 246: note = Ile, not present or Met
  VARIANT 247: note = Lys, not present or Ser
  VARIANT 248: note = Asn, not present or Gly
  VARIANT 249: note = Glu or not present
  VARIANT 250: note = Lys or Ser
  VARIANT 251: note = Phe, Tyr or Ile
  VARIANT 252: note = Lys, Asn or Gly
  VARIANT 253: note = Asn or Thr
  VARIANT 254: note = Met, Thr or Ala
  VARIANT 255: note = Asn or Ser
  VARIANT 256: note = Ile, Ser or Gly
  VARIANT 257: note = Lys, Arg or Ala
  VARIANT 258: note = Lys, Leu or Ser
  VARIANT 259: note = Val, Asn or Gly
  VARIANT 260: note = Asn, Leu or Gly
  VARIANT 261: note = Thr, Asn or Ala
  VARIANT 262: note = Lys, Thr or Pro
  VARIANT 263: note = Asn or Met
  VARIANT 264: note = Cys, Lys or Ala
  VARIANT 265: note = Asn or Thr
  VARIANT 266: note = Ile or Pro
  VARIANT 267: note = Thr, Pro or Asn
  VARIANT 268: note = Asn, Thr or Gly
  VARIANT 269: note = Ser or Ala
  VARIANT 270: note = Lys, not present or Thr
  VARIANT 271: note = Ala, not present or Thr
  VARIANT 272: note = Lys, not preesnt or Leu
  VARIANT 273: note = Lys, not present or Gly
  VARIANT 274: note = Thr, not present or Gly
  VARIANT 275: note = Asp, not present or Ser
  VARIANT 276: note = Asn or not present
  VARIANT 277: note = Asn, not present or Gly
  VARIANT 278: note = Ile, not present or Met
  VARIANT 279: note = Not present or Gly
  VARIANT 280: note = Gln, not present or Ser
  VARIANT 281: note = Asn or not present
  VARIANT 282: note = Asp, not present or Ser
  VARIANT 283: note = Met, not present or Val
  VARIANT 284: note = Asn, not present or Gly
  VARIANT 286: note = Asn or Gly
  VARIANT 287: note = His, Glu or Ser
  VARIANT 288: note = Pro, Asn or Leu
  VARIANT 289: note = Asn, Arg or Ala
  VARIANT 290: note = Asn or His
  VARIANT 292: note = Phe, Val or Ser
  VARIANT 293: note = Val or Asn
  VARIANT 294: note = Asn, Ser or Gly
  VARIANT 295: note = Asn or Ser
  VARIANT 296: note = Asn or Ser
  VARIANT 297: note = Ile or Val
  VARIANT 298: note = Leu, Gln or Gly
  VARIANT 299: note = Asp or Ser
  VARIANT 300: note = Asn, Lys or Ser
  VARIANT 301: note = Asn, Ile or Val
  VARIANT 302: note = His, Asn or Gly
  VARIANT 303: note = Not present or Ser
  VARIANT 304: note = Not present or Ser
  VARIANT 305: note = Not present or Val
  VARIANT 306: note = Not present or Gly
  VARIANT 307: note = Not present or Ser
  VARIANT 308: note = Not present or Ser
  VARIANT 309: note = Not present or Val
  VARIANT 310: note = Not present or Gly
  VARIANT 311: note = Not present or Ser
  VARIANT 312: note = Not present or Gly
  VARIANT 313: note = Not present or Val
  VARIANT 314: note = Not present or Gly
  VARIANT 315: note = Not present or Ser
  VARIANT 316: note = Not present or Gly
  VARIANT 317: note = Ile or Val
  VARIANT 318: note = Asn, Thr or Gly
  VARIANT 320: note = Asn, Tyr or Gly
  VARIANT 321: note = Ile or Val
  VARIANT 322: note = Leu, Asn or Gly
  VARIANT 323: note = Asp or Ser
  VARIANT 324: note = Asn or Gly
  VARIANT 325: note = Not present or Val
  VARIANT 326: note = Not present or Gly
  VARIANT 327: note = Not present or Ser
  VARIANT 328: note = Not present or Gly
  VARIANT 329: note = Not present or Val
  VARIANT 330: note = Not present or Gly
  VARIANT 332: note = His, not present or Gly
  VARIANT 333: note = Ile, not present or Val
  VARIANT 334: note = Asn, not present or Gly
  VARIANT 335: note = Asn, not present or Ser
  VARIANT 336: note = Asn, Thr or Gly
  VARIANT 337: note = Ile or Val
  VARIANT 338: note = Leu, Thr or Gly
  VARIANT 339: note = Asp, Ile or Asn
  VARIANT 340: note = Asn or Gly
  VARIANT 341: note = Asn or Ser
  VARIANT 342: note = His, Thr or Leu
  VARIANT 343: note = Ile or Ala
  VARIANT 345: note = Asn or Lys
  VARIANT 346: note = Asn, Glu or Ser
  VARIANT 347: note = Not present or Ala
  VARIANT 348: note = Not present or Gly
  VARIANT 349: note = His or Glu
  VARIANT 350: note = Leu or Ser
  VARIANT 351: note = Asn, Cys or Ala
  VARIANT 352: note = Asn, Gln or Arg
  VARIANT 353: note = Asn, Tyr or Met
  VARIANT 354: note = His or Asn
  VARIANT 355: note = Leu, Thr or Ser
  VARIANT 356: note = Asn, Lys or Gly
  VARIANT 357: note = Asn, Phe or Glu
  VARIANT 358: note = Asn or Ala
  VARIANT 359: note = His, Ser or Asn
  VARIANT 360: note = Leu or not present
  VARIANT 361: note = Asn or Gly
  VARIANT 362: note = Asn or Gly
  VARIANT 364: note = His, Asn or Pro
  VARIANT 365: note = Leu, Thr or Ser
  VARIANT 366: note = Asn, Thr or Ser
  VARIANT 367: note = Asn or Gly
  VARIANT 368: note = Asn, Ser or Gly
  VARIANT 369: note = His, Ile or Thr
  VARIANT 370: note = Leu, Val or Pro
  VARIANT 371: note = Asn, Lys or His
  VARIANT 373: note = Asn, Phe or Gly
  VARIANT 374: note = His or Cys
  VARIANT 375: note = Phe or Leu
  VARIANT 376: note = Asn or Ser
  VARIANT 377: note = Asn or Ala
  VARIANT 378: note = Asn or Ser
  VARIANT 379: note = His, not present or Ala
  VARIANT 380: note = Leu, not present or Ala
  VARIANT 381: note = Asn, not present or Ala
  VARIANT 382: note = Asn, not present, or Met
  VARIANT 383: note = Asn or not present
  VARIANT 384: note = Gln, not present or Asp
  VARIANT 385: note = Leu, not present or Ala
  VARIANT 386: note = Asn, not present or Gly
  VARIANT 388: note = His, not present or Gly
  VARIANT 387: note = Asn, not present or Thr
  VARIANT 389: note = Leu, not present or Gln
  VARIANT 390: note = Leu, not present or His
  VARIANT 391: note = Asp, not present or His
  VARIANT 392: note = Asn, not present or His
  VARIANT 393: note = Ar, not present or Gln
  VARIANT 394: note = Asp, not present or Gln
  VARIANT 395: note = Lys, not present or Arg
  VARIANT 397: note = Gln, Glu or Cys
  VARIANT 399: note = Leu or Tyr
  VARIANT 400: note = His, not present or Asn
  VARIANT 401: note = Tyr, not present or Ser
  VARIANT 402: note = Lys or not present
  VARIANT 403: note = His, not present or Asn
  VARIANT 404: note = Ala or not present
  VARIANT 405: note = Asn or Ser
  VARIANT 406: note = Ser or Thr
  VARIANT 407: note = Asn, Ser or Gly
  VARIANT 408: note = Ser or Ala
  VARIANT 409: note = Not present or Gly
  VARIANT 410: note = Not present or Ala
  VARIANT 411: note = Not present or His
  VARIANT 412: note = Not present or Ser
  VARIANT 413: note = Not present or Phe
  VARIANT 414: note = Not present or Leu
  VARIANT 415: note = Not present or Lys
  VARIANT 416: note = Not present or Ser
  VARIANT 417: note = Not present or Ser
  VARIANT 418: note = Not present or Cys
  VARIANT 419: note = Not present or Tyr
  VARIANT 420: note = Val, Leu or Ile
  VARIANT 421: note = Leu or Gly
  VARIANT 422: note = Lys, Cys or Ala
  VARIANT 423: note = Tyr, Asp or Gly
  VARIANT 424: note = Asn or Glu
  VARIANT 425: note = Asn or Ala
  VARIANT 426: note = Ser, Ile or Cys
  VARIANT 427: note = Asn or Thr
  VARIANT 428: note = Asn, Thr or Ala
  VARIANT 429: note = Asn or Gly
  VARIANT 430: note = Tyr, Asp or His
  VARIANT 432: note = Ala, Val or Ser
  VARIANT 433: note = Ser or His
  VARIANT 434: note = Gln, Asn or Gly
  VARIANT 435: note = Asn or Ser
  VARIANT 436: note = Gln, Val or His
  VARIANT 437: note = Ile, Thr or Asn
  VARIANT 438: note = Phe, Gly or Asn
  VARIANT 439: note = Phe, Ser or His
  VARIANT 440: note = Ser or Asn
  VARIANT 441: note = Glu, Lys or Asn
  VARIANT 442: note = Glu, Ser or His
  VARIANT 443: note = Elu, Ile or Asn
  VARIANT 445: note = Arg, Asn or His
  VARIANT 446: note = Thr, Pro or Asn
  VARIANT 447: note = His, not present or Asn
  VARIANT 448: note = Phe, not present or His
  VARIANT 449: note = Leu, not present or Asn
  VARIANT 450: note = Glu or not present
  VARIANT 451: note = Asn or Pro
  VARIANT 453: note = Pro, Tyr or Gly
  VARIANT 454: note = Asn or Lys
  VARIANT 455: note = Lys or Asn
  VARIANT 456: note = Arg, Lys or Pro
  VARIANT 460: note = Ile or Val
  VARIANT 461: note = Ser, Asn or Ala
  VARIANT 472: note = Tyr or Trp
  VARIANT 478: note = Ile or Met
  VARIANT 479: note = Lys or Arg
  VARIANT 481: note = Ser or Ala
  VARIANT 482: note = Glu or Lys
  VARIANT 485: note = Not present or Asn
  VARIANT 486: note = Phe or Tyr
  VARIANT 487: note = Leu, Ser or Val
  VARIANT 489: note = Glu or Asp
  VARIANT 490: note = Asn or His
  VARIANT 491: note = Pro, Ser or Thr
  VARIANT 492: note = Asp or Glu
  VARIANT 495: note = Val or Ile
  VARIANT 496: note = Glu or Asp
  VARIANT 497: note = Asn or Lys
  VARIANT 498: note = Val or Ile
  VARIANT 500: note = Lys or Gln
  VARIANT 501: note = Leu, Ile or Val
  VARIANT 503: note = His or Tyr
  VARIANT 507: note = Gln, Leu or Val
  VARIANT 514: note = Asn or Arg
  VARIANT 516: note = Asn or Lys
  VARIANT 517: note = Asn or Ser
  VARIANT 518: note = Ile or Leu
  VARIANT 519: note = Asp, Leu or Val
  VARIANT 520: note = Gly or Asp
  VARIANT 521: note = Gly, Ile or Gln
  VARIANT 522: note = Tyr, Ser or Thr
  VARIANT 523: note = Gly or Asn
  VARIANT 524: note = Asn or Ser
  VARIANT 526: note = Asn or Ser
  VARIANT 527: note = Leu, Thr or Arg
  VARIANT 528: note = Cys or Pro
  VARIANT 529: note = Ala, Thr or Asn
  VARIANT 530: note = Pro, Ile or Phe
  VARIANT 531: note = Thr or Ser
  VARIANT 532: note = Ser or Asn
  VARIANT 533: note = Leu or Phe
  VARIANT 535: note = Leu or Pro
  VARIANT 536: note = His, Phe or Met
  VARIANT 537: note = Asn or Tyr
  VARIANT 539: note = Glu or Asn
  VARIANT 540: note = Lys or Gln
  VARIANT 541: note = Gly, Asn or not present
  VARIANT 542: note = Asn or not present
  VARIANT 543: note = Thr, Asn or not present
  VARIANT 544: note = Ile or not present
  VARIANT 545: note = Lys, Thr or not present
  VARIANT 546: note = Asn, Met or not present
  VARIANT 547: note = Asp, Glu or not present
  VARIANT 548: note = Va, Thr or not present
  VARIANT 549: note = Asn or not present
  VARIANT 550: note = Ile, Ser or not present
  VARIANT 551: note = Asn or not present
  VARIANT 552: note = Asn, Pro or not present
  VARIANT 554: note = Ser, Asn or not present
  VARIANT 555: note = Asn, Thr or not present
  VARIANT 556: note = Asn, Ser or not present
  VARIANT 557: note = Asn, Gly or not present
  VARIANT 558: note = Asn, Thr or not present
  VARIANT 559: note = Asn, Ala or not present
  VARIANT 560: note = Asn, Thr or not present
  VARIANT 561: note = Asn, Thr or not present
  VARIANT 562: note = Tyr or not present
  VARIANT 563: note = Asn or not present
  VARIANT 564: note = Tyr or not present
  VARIANT 565: note = Asn or not present
  VARIANT 566: note = Asn or not present
  VARIANT 567: note = Asn or not present
  VARIANT 568: note = Asn or not present
  VARIANT 569: note = Tyr or not present
  VARIANT 570: note = Asn, Ser or not present
  VARIANT 571: note = Asn or not present
  VARIANT 572: note = Asn, Thr or not present
  VARIANT 573: note = Asn, Ala or not present
  VARIANT 574: note = Ile, Thr or not present
  VARIANT 575: note = Asn, Ala or not present
  VARIANT 576: note = Tyr, Ser and not present
  VARIANT 577: note = Asn, Thr and not present
  VARIANT 578: note = Asn, Ala or not present
  VARIANT 579: note = Phe, Thr or not present
  VARIANT 580: note = Ser, Ala or Thr
  VARIANT 582: note = Asn, Asp pr Ser
  VARIANT 583: note = Asn or Val
  VARIANT 584: note = Gln or Asn
  VARIANT 586: note = Lys or Arg
  VARIANT 587: note = Ile, Pro or Met
  VARIANT 590: note = Thr, Ser or Ala
  VARIANT 591: note = Lys or Asn
  VARIANT 592: note = Met or Leu
  VARIANT 594: note = Gln, Ala or Glu
  VARIANT 595: note = Ala, Gln or Asn
  VARIANT 597: note = Val, Ile or Met
  VARIANT 599: note = His, Thr or Ala
  VARIANT 601: note = Thr, not present or His
  VARIANT 602: note = Arg or not present
  VARIANT 603: note = Thr or not present
  VARIANT 604: note = Ser or not present
  VARIANT 605: note = Asn or not present
  VARIANT 606: note = Leu or not present
  VARIANT 607: note = Asn, not present or Ser
  VARIANT 608: note = Asn, not present or Arg
  VARIANT 609: note = Asn or Pro
  VARIANT 610: note = Asn, Lys or Ile
  VARIANT 611: note = Asn, Gln or Glu
  VARIANT 612: note = Asn or Thr
  VARIANT 613: note = Asn or Glu
  VARIANT 614: note = Asn, Ile or Leu
  VARIANT 615: note = Asn or Ala
  VARIANT 617: note = Thr, Pro or Ser
  VARIANT 619: note = Asn, Ser or Ala
  VARIANT 620: note = Thr, Ser or Val
  VARIANT 622: note = Met, Ile or Val
  VARIANT 624: note = Tyr, Thr or Asn
  VARIANT 626: note = Met, Phe or Ile
  VARIANT 627: note = Asn or Ile
  VARIANT 629: note = Pro, Gln or His
  VARIANT 631: note = Asp, Asn or Thr
  VARIANT 632: note = Met, Ile or Leu
  VARIANT 633: note = Ser or Thr
  VARIANT 634: note = Thr or Leu
  VARIANT 635: note = Thr or Ser
  VARIANT 637: note = Thr or not present
  VARIANT 638: note = His or not present
  VARIANT 639: note = Met or not present
  VARIANT 640: note = Thr or not present
  VARIANT 641: note = Asn or not present
  VARIANT 642: note = Leu, Thr or Cys
  VARIANT 643: note = Ser, Asn or Ala
  VARIANT 644: note = Thr, Gly or Ala
  VARIANT 645: note = Ile or Ala
  VARIANT 646: note = Asn, Thr or Gln
  VARIANT 649: note = Asp, Pro or Val
  VARIANT 650: note = Gln, Arg or Ser
  VARIANT 651: note = Asn, Thr or Ala
  VARIANT 652: note = Lys, Cys or Thr
  VARIANT 653: note = Asn, Ile or Pro
  VARIANT 654: note = Asn, Leu or Ser
  VARIANT 655: note = Asn or Cys
  VARIANT 656: note = Met, Pro or Ser
  VARIANT 657: note = Asn, His or Ser
  VARIANT 658: note = Asn or Gly
  VARIANT 659: note = Asp, Asn or Gly
  VARIANT 660: note = Thr, Asn or Ser
  VARIANT 661: note = Asp, Asn or Gly
  VARIANT 662: note = Ser, Asn or Gly
  VARIANT 663: note = Asn or Ser
  VARIANT 664: note = Ile or Ser
  VARIANT 665: note = Tyr, Glu or Gly
  VARIANT 666: note = Ile, Ala or Ser
  VARIANT 667: note = Not present, Gly or Ala
  VARIANT 668: note = Asn, Asp or Gly
  VARIANT 670: note = Lys, Pro or Ser
  VARIANT 671: note = Ser or Gly
  VARIANT 672: note = Asn or Gly
  VARIANT 673: note = Asp, Asn or Ser
  VARIANT 674: note = Asn or Gly
  VARIANT 675: note = Asn or Gly
  VARIANT 676: note = Ile, Thr or Ser
  VARIANT 677: note = Ile or Ser
  VARIANT 678: note = Asn, Ser or Gly
  VARIANT 679: note = Asn or Ser
  VARIANT 680: note = Ile, Asn or Ala
  VARIANT 681: note = Asn or Gly
  VARIANT 683: note = Met, Thr or Ser
  VARIANT 684: note = Asn or Gly
  VARIANT 685: note = Ile, Cys or Gly
  VARIANT 686: note = Met or Ser
  VARIANT 687: note = Asn, Glu or Gly
  VARIANT 688: note = Asn or Gly
  VARIANT 689: note = Ile or Ser
  VARIANT 691: note = Asn, Thr or Arg
  VARIANT 692: note = Ile or Thr
  VARIANT 693: note = Asn, Thr or Gly
  VARIANT 695: note = Ile or Asp
  VARIANT 696: note = Lys, Asn or Ser
  VARIANT 697: note = Asn or Ala
  VARIANT 698: note = Ile, Asn or Ser
  VARIANT 700: note = Asn or Ser
  VARIANT 701: note = Ile or not present
  VARIANT 702: note = Asn or not present
  VARIANT 703: note = Asn or not present
  VARIANT 704: note = Ile or not present
  VARIANT 705: note = Lys or not present
  VARIANT 706: note = Asn or not present
  VARIANT 707: note = Ile or not present
  VARIANT 708: note = Asn or not present
  VARIANT 709: note = Asn or not present
  VARIANT 710: note = Ile or not present
  VARIANT 711: note = Asn or not present
  VARIANT 712: note = Asn, Glu or not present
  VARIANT 713: note = Ile, Thr or not present
  VARIANT 714: note = Asn or Thr
  VARIANT 715: note = Ser or Asp
  VARIANT 716: note = Ile, Lys or Gly
  VARIANT 717: note = Asn, Lys or Glu
  VARIANT 718: note = Ser or Gly
  VARIANT 719: note = Ile, Arg or Ala
  VARIANT 720: note = Asn or Pro
  VARIANT 722: note = Met, Lys or Glu
  VARIANT 723: note = Asn or Ser
  VARIANT 724: note = His, Ile or Gly
  VARIANT 725: note = Met, Asn or Pro
  VARIANT 727: note = His, Lys or Gly
  VARIANT 728: note = Ile or Thr
  VARIANT 729: note = Asn, Ile or Ser
  VARIANT 730: note = Gly, not present or Pro
  VARIANT 731: note = Asn, not present or Thr
  VARIANT 733: note = Asn, Glu or Ile
  VARIANT 734: note = Ile or Ser
  VARIANT 735: note = Asn or Glu
  VARIANT 736: note = Ser, Thr or Leu
  VARIANT 737: note = Asn or Thr
  VARIANT 738: note = Asn, Asp or Ser
  VARIANT 739: note = Asn or Val
  VARIANT 740: note = Ile or Thr
  VARIANT 742: note = Ser, not present or Leu
  VARIANT 743: note = Asn, not present or Thr
  VARIANT 745: note = Asn, Ser or Ala
  VARIANT 746: note = Ile or not present
  VARIANT 747: note = Asn or not present
  VARIANT 748: note = Ser or Pro
  VARIANT 749: note = Asn or Ser
  VARIANT 751: note = Asn, His or Gly
  VARIANT 752: note = Ile, Asn or Ser
  VARIANT 754: note = Ser or Ile
  VARIANT 756: note = Asn or Ala
  VARIANT 757: note = Asn or Thr
  VARIANT 758: note = Ile or Met
  VARIANT 759: note = Asn or Glu
  VARIANT 760: note = Ser or Thr
  VARIANT 761: note = Asn, Ile or Asp
  VARIANT 762: note = Asn or Ser
  VARIANT 763: note = Asn or Pro
  VARIANT 766: note = Ser or not present
  VARIANT 767: note = Asn or not present
  VARIANT 768: note = Asn or not present
  VARIANT 769: note = Asn or not present
  VARIANT 770: note = Ile or not present
  VARIANT 771: note = Leu or not present
  VARIANT 772: note = Asn or not present
  VARIANT 773: note = Asn or not present
  VARIANT 774: note = Thr or not present
  VARIANT 775: note = Asn or not present
  VARIANT 776: note = Glu or not present
  VARIANT 777: note = Arg or not present
  VARIANT 778: note = Asn or not present
  VARIANT 779: note = Lys or not present
  VARIANT 780: note = Tyr or not present
  VARIANT 781: note = Asn or not present
  VARIANT 782: note = Ile or not present
  VARIANT 783: note = Glu or not present
  VARIANT 784: note = Asn or not present
  VARIANT 785: note = Glu or not present
  VARIANT 786: note = Tyr or not present
  VARIANT 787: note = Ser or not present
  VARIANT 788: note = Lys or not present
  VARIANT 789: note = Asn or not present
  VARIANT 790: note = Ile or not present
  VARIANT 791: note = Gln, Leu or not present
  VARIANT 792: note = Asn, Asp or not present
  VARIANT 793: note = Ile or not present
  VARIANT 794: note = Gln, Glu or not present
  VARIANT 796: note = Ile or Phe
  VARIANT 797: note = Gln, Asn or Ser
  VARIANT 798: note = Asn or Thr
  VARIANT 799: note = Val, Tyr or Ile
  VARIANT 800: note = Gln or Asn
  VARIANT 802: note = Asn, Met or Arg
  VARIANT 803: note = Gln or Asn
  VARIANT 804: note = Ser, Cys or not present
  VARIANT 805: note = Tyr, Asn or not present
  VARIANT 806: note = Val or Ile
  VARIANT 810: note = Tyr or His
  VARIANT 811: note = Tyr, Ala or Ser
  VARIANT 813: note = Asn or Ala
  VARIANT 814: note = Arg or Cys
  VARIANT 815: note = His or Gln
  VARIANT 816: note = Asn, not present or Ile
  VARIANT 817: note = Ile, Val or Thr
  VARIANT 818: note = Asn or Cys
  VARIANT 819: note = Ser, Cys or Val
  VARIANT 820: note = Ser, Leu or Pro
  VARIANT 821: note = Thr or Pro
  VARIANT 823: note = Leu or Pro
  VARIANT 824: note = Asp, Gln or Cys
  VARIANT 825: note = Thr or Ser
  VARIANT 826: note = Ser, Asp or Thr
  VARIANT 827: note = Thr or Arg
  VARIANT 829: note = Asp or Tyr
  VARIANT 830: note = Gln, Thr or Ser
  VARIANT 831: note = Lys or Arg
  VARIANT 832: note = Asn, Leu or Met
  VARIANT 833: note = Lys, Thr or Asn
  VARIANT 834: note = Ser, Phe or His
  VARIANT 835: note = Tyr or Phe
  VARIANT 838: note = Asp, Asn or Gln
  VARIANT 839: note = Asn or Glu
  VARIANT 840: note = Lys or Asn
  VARIANT 841: note = Glu or Asn
  VARIANT 843: note = Met or Leu
  VARIANT 844: note = Tyr or Cys
  VARIANT 845: note = Glu, Leu or Arg
  VARIANT 847: note = Asn or Lys
  VARIANT 848: note = Lys or Gln
  VARIANT 849: note = Lys, Asn or Thr
  VARIANT 850: note = Asp, Asn or Glu
  VARIANT 851: note = Glu or Asp
  VARIANT 852: note = His or Phe
  VARIANT 853: note = Glu, not present or Pro
  VARIANT 854: note = Asp or not present
  VARIANT 855: note = Lys or not present
  VARIANT 856: note = Asn or not present
  VARIANT 857: note = Gln or not present
  VARIANT 858: note = Tyr or not present
  VARIANT 859: note = His or not present
  VARIANT 860: note = Asp or not present
  VARIANT 861: note = His or not present
  VARIANT 862: note = Gln or not present
  VARIANT 863: note = Gly or not present
  VARIANT 864: note = Lys or not present
  VARIANT 865: note = Gln or not present
  VARIANT 866: note = Gln or not present
  VARIANT 867: note = Gln or not present
  VARIANT 868: note = Gln or not present
  VARIANT 869: note = Gln or not present
  VARIANT 870: note = Gln or not present
  VARIANT 871: note = Gln or not present
  VARIANT 872: note = Gln or not present
  VARIANT 873: note = Gln or not present
  VARIANT 874: note = Gln or not present
  VARIANT 875: note = Gln or not present
  VARIANT 876: note = Gln or not present
  VARIANT 877: note = Gln or not present
  VARIANT 878: note = Gln or not present
  VARIANT 879: note = Gln or not present
  VARIANT 880: note = Gln or not present
  VARIANT 881: note = Gln or not present
  VARIANT 882: note = Gln or not present
  VARIANT 883: note = Gln or not present
  VARIANT 884: note = His or not present
  VARIANT 885: note = His or not present
  VARIANT 886: note = Asn or not present
  VARIANT 887: note = Asn or not present
  VARIANT 888: note = Asn or Thr
  VARIANT 889: note = Asn or Met
  VARIANT 890: note = Ile, Leu or Gln
  VARIANT 891: note = Ser or Val
  VARIANT 892: note = His, Thr or Asn
  VARIANT 893: note = Thr, Ser or Asn
  VARIANT 895: note = Asn or Thr
  VARIANT 896: note = Asn or Ser
  VARIANT 899: note = Asn or Thr
  VARIANT 903: note = Gln or Asp
  VARIANT 904: note = Phe or Ile
  VARIANT 905: note = Leu or Val
  VARIANT 908: note = Thr or Ala
  VARIANT 915: note = Thr or Ser
  VARIANT 917: note = Val or not present
  VARIANT 918: note = Asn or not present
  VARIANT 922: note = Ala, Thr or Asn
  VARIANT 927: note = Ser or Thr
  VARIANT 928: note = Leu or Val
  VARIANT 931: note = Ile or Phe
  VARIANT 932: note = Cys or Tyr
  VARIANT 933: note = Lys or Arg
  VARIANT 934: note = Asn, Ala or Thr
  VARIANT 936: note = Ser or Ala
  VARIANT 941: note = Leu or Ile
  VARIANT 942: note = Lys, Gly or Arg
  VARIANT 945: note = Val or Ile
  VARIANT 948: note = Met, Phe or Leu
  VARIANT 949: note = Glu or Asp
  VARIANT 553: note = Not present, Ile or Val
XXXXKXEXXXXXXBKXXXXXXXXXXKXXXXXXXXXXXXXXXXXVXXVYFVNYRAVVVKTKXYYXTQNSYXLFGDVFKIYNLKGEIKKIHAFFPGDZNNLSDTLIMLKTTXXAVTTSKXEALRXADXIXMXCKICXKKRKATKFSIEGFLRKVRCDTCRVKPXXXIXKXXXXXXXXXXXXXXXXXXXXXXNBXXXXXXXXXXXXXXXXXXXXXXXXXPXXXXXXXXXNXXXXXXXXXXXXXXXXXXXXXXXXXXXXXXXXXXXXXXXXXXXXXXXXXXXXXXXXXNXXXXXNXXXXXXXXXXXXXXXXXXXXXXXXXXXNXXXXXXXXXXXNXXXXXXXXXXXXNXXXXXXXXXXXXXXXXXXNXXXXXXXXNXXXXXXXXXXXXXXXXXXXXXXXIXSXXXXXXXXXXXXXXXXXXXXXXXXXXXXXXXXSXXXXXXXXXXXXNXXXXXXXDXXXXDEIXXMIIELIKYISXMQKALXXAXXGNXXXIXXXXINXXXXSXXVXSAQXLCNKLLXQXXJXXXXXXNXXXXXXXXJXXXNXXXXXXXXXXXXXXXXXXXXXXXXXXXXXXXXXXXXXXXXXXNXXXJXXKYXXXYXXKXNHIXXXXXXXXXXXXXXXNXNXXBXFXNXXNXNXXXXXNXXXXXXXXXXRNXXXXXXXXXXXXXXXXXXXXNXXXXXXXXXXXXNXXXXXXXNXXXNXXXXNXXXXXXXXXXXXXXXXXXXXXNXXXXNXXXXXNXXXXXXXXNXXNXXXXXNXXBXNXXXXXXXXJNXXXXXXXXXXXXXXXXXXXXXXXXXXXXXNXXXXXNXXXXXJPYXXNXXXXXXXXXSXXXXXNXXXXXXXIKXXXXYXXXNXXXXXXXXXXXXXXXXXXXXXXXXXXXXXXXXXXXXXXXXXXXXXXXKXXYYXBKQXXXFSXLSKQYNXNXXISFXNINNXXSNXXXXVXKNYNXXDTXLNXXK

SEQ ID NO: 5 - DNA, 36 bp, synthetic construct
  source 1..36: mol_type = other DNA
ataagcttgtttgtatcttggattttgaaatggttg

SEQ ID NO: 6 - DNA, 60 bp, synthetic construct
  source 1..60: mol_type = other DNA
aatagcattgaccaagctagctataggcgcgccttttggatcaaacgtggggtatatatg

SEQ ID NO: 7 - DNA, 60 bp, synthetic construct
  source 1..60: mol_type = other DNA
tccaaaaggcgcgcctatagctagcttggtcaatgctattatatatttttattacttccc

SEQ ID NO: 8 - DNA, 40 bp, synthetic construct
  source 1..40: mol_type = other DNA
tagcggccgctaaccattacatattcgataatgtaaacag

SEQ ID NO: 9 - DNA, 24 bp, synthetic construct
  source 1..24: mol_type = other DNA
tattcgatggagttgtctctgctt

SEQ ID NO: 10 - DNA, 24 bp, synthetic construct
  source 1..24: mol_type = other DNA
aaacaagcagagacaactccatcg

SEQ ID NO: 11 - DNA, 27 bp, synthetic construct
  source 1..27: mol_type = other DNA
tttatagctcattattctggcatacac

SEQ ID NO: 12 - DNA, 27 bp, synthetic construct
  source 1..27: mol_type = other DNA
gcaataaaataagcagaatgcgtatgc

SEQ ID NO: 13 - DNA, 47 bp, synthetic construct
  source 1..47: mol_type = other DNA
gaatatgtaatggttagggcccacaataatactccaaccatctcttc

SEQ ID NO: 14 - DNA, 35 bp, synthetic construct
  source 1..35: mol_type = other DNA
atactagttttctcaaagtttaaaatagtatctcc

SEQ ID NO: 15 - DNA, 39 bp, synthetic construct
  source 1..39: mol_type = other DNA
atccgcggtcaatgctattatatatttttattacttccc

SEQ ID NO: 16 - DNA, 52 bp, synthetic construct
  source 1..52: mol_type = other DNA
gttggagtattattgtgggccctaaccattacatattcgataatgtaaacag

SEQ ID NO: 17 - DNA, 27 bp, synthetic construct
  source 1..27: mol_type = other DNA
aaaagctttaataaaagcagccaaagg

SEQ ID NO: 18 - DNA, 27 bp, synthetic construct
  source 1..27: mol_type = other DNA
ttctccttctatttcaaattcatgtcc

SEQ ID NO: 19 - DNA, 25 bp, synthetic construct
  source 1..25: mol_type = other DNA
atgagaagaatgattaaggatcccg

SEQ ID NO: 20 - DNA, 27 bp, synthetic construct
  source 1..27: mol_type = other DNA
gcaataaaataagcagaatgcgtatgc

SEQ ID NO: 21 - DNA, 24 bp, synthetic construct
  source 1..24: mol_type = other DNA
tattgttgttgttgtttgccttgg

SEQ ID NO: 22 - DNA, 24 bp, synthetic construct
  source 1..24: mol_type = other DNA
aaacccaaggcaaacaacaacaac

SEQ ID NO: 23 - DNA, 24 bp, synthetic construct
  source 1..24: mol_type = other DNA
tattatatgtgctactccatatac

SEQ ID NO: 24 - DNA, 24 bp, synthetic construct
  source 1..24: mol_type = other DNA
aaacgtatatggagtagcacatat

SEQ ID NO: 25 - DNA, 37 bp, synthetic construct
  source 1..37: mol_type = other DNA
atactagtttttattttttatatgaaccatttaagac

SEQ ID NO: 26 - DNA, 60 bp, synthetic construct
  source 1..60: mol_type = other DNA
tacttttccccaacccgggtataggcgcgcctctttcaacaaaaaataatatatgtgtac

SEQ ID NO: 27 - DNA, 60 bp, synthetic construct
  source 1..60: mol_type = other DNA
ttgttgaaagaggcgcgcctatacccgggttggggaaaagtaaaattcgcgataaaaatc

SEQ ID NO: 28 - DNA, 38 bp, synthetic construct
  source 1..38: mol_type = other DNA
tagcggccgcaatatatatgtgattttattaagtacgg

SEQ ID NO: 29 - DNA, 26 bp, synthetic construct
  source 1..26: mol_type = other DNA
taaatgagggataaaacctcaaaaag

SEQ ID NO: 30 - DNA, 27 bp, synthetic construct
  source 1..27: mol_type = other DNA
aatatgtttcaaacggttaaatgtaag

SEQ ID NO: 31 - DNA, 28 bp, synthetic construct
  source 1..28: mol_type = other DNA
cttaacaaccatcttcttgacaataggg

SEQ ID NO: 32 - DNA, 27 bp, synthetic construct
  source 1..27: mol_type = other DNA
gttatgtgattaactttcgcttgatac

SEQ ID NO: 33 - DNA, 1446 bp, Plasmodium yoelii
  source 1..1446: mol_type = genomic DNA
atgaatatgaatttgaaaaatataaacttagaagacgaaaaaataaataaaagagtgagtaataaaagaatagtgcatataaaaaatacatatatatctccatatgttctttataataaaaaggggaatagtaataatcaattggataaattaaataaaatatttactagtgaaaataattgtagattttcaaaagaatattataattatgttaaagatggtgatatatgtaatagtggagaaaaatatcctgaatgttgttccccaaatgttaagcaaaacaatagcccaaattatgaccaatgtaataatgaaaaaaaaatattaaaatatggcttgttgaatgaacaaaatatgaaatttttaaaaaaaaaaaaagaagaagattttgaattaaatacaatatctactatgatacatatttattctgattatgaaagtgataaagacaatgaaacgataacaaatgaatataatgatttaatagattgtataaaaaaaataaatatttctaataataaatgtgttattaataatgataaaaaaatagaggatactttattaagtccttatgaacagtatagaaaatgtagttatcagacaattgggcataaagaaaattatagtaacaaaaataatgaaaaaaatagtgacaaaaataatgaaaaaaatagtgacaaaaataatgaaaaaaatagtgacaacaatagcgataataataatgataacaatagcgacaataataataacagaaatagcgataataataatagtgatgataatggagaaaaatgtgtgcaaagattaaataattttatatctattccttcagtatcaaacaaaagttgcatagaacatgtacatgttcagaatgatcatatggatactgtgtattatgataattttatttgtaataaagaaaatcgtgataataatgaaaatgatactatatataaatgtgaagaatctataccacttggaacaatattaaatattcataatttagatagtaataataataatggtctaacaacagttatgttaagaaatataccaaataaatatacacaaaatatgttaatggatgttatgaatgagcattttaaaggtttatatgattttttttatttaccaattgattttcgtaacaaatgtaatgttggttatgcatttattaattttatacatcctcattatgctgaactatttattaaattttttaataattataaattaaatgcatttaaaagtaataaaatatgcactgttacatggggaagggtacaaggattaaaagctaatattgaacattacagaaattctgcaattatgactatatctgtgcctcaatataagcctatactttttcaaaatggaatatctgtttcatggcctgaatcggatggtcccttaccagctataaaacttcgatcacataaatattag

SEQ ID NO: 34 - DNA, 1284 bp, Plasmodium falciparum
  source 1..1284: mol_type = genomic DNA
atgcataagatagatgaaaagataaatatgaataatgtaaaaggcactagaattattcatgtaaaaaatacatatatatctccatacatactacacaatataaacaataatgaaggcccattaaaaaatatagacaaaatattaaaagaagaaaaagataatataaatttaaagatcaataatgttaataataaggaaactaataatgataagcacacattaaaatattcaacattatgtaatgatctaaatataatgcatacacaaaataaagaagaagaaattgaattaaatacaatttcaacggttatacacatgaattctgatgaagatagtgataaagataatgataccataataaatgaaaacaacgatttgttaattgataatataaagaaatatcatgccgttaaagataatcataataatattatttataatgataaatataatacaattaataataattcagtcattaatgatgtatgtaattctattcattttaataataattcatatataactaactttaatttaaatcataatttttctgtgtgtaatcgtactttaaataacacctgcacaagtcaggtaaatttgagaaataatatgaacagcaataaaaaaactaatgataacaataaaaatctgaacaatgaaattaataagaaaatcaataatgataatattataaacgaatttgataatattaataataagaaaaataattatattgattgttccgtttacaaatgtgaggatgaaataccccttggtaccatattaaatatccaagatttggatatacataatagaaataatatgaacaattgtaataataatattaataataagagtaagattcttacaacagttatgcttagaaatattccaaacaaatatacacaaaatatgttgatggatgttatgaatgaacattttaaaggtttatatgattttttttatttaccaattgattttagaaataaatgtaatgttggatatgcttttattaatttcatacatccatattatgctgaattgtttatcaaattttttaataattataaactcaatgcatttaaaagtaacaaagtctgttctgttacttggggaagagtacaaggattaaaggcaaatattgagcattatagaaattcagcgattatgaccataccaataccacaatataaacctatactttttcaaaatggtataactgtttcgtggcctgaatcagatgggcctttaccatccatcaaattaagatctcaaaaattttaa

SEQ ID NO: 35 - DNA, 2727 bp, Plasmodium falciparum
  source 1..2727: mol_type = other DNA
atgaaaaatgaagattctacatataaagaagataagtattcaaaatattcaaaagagaatatccaaaaaatatgtgctactccatataccggatctaatataagtaacacagtagaatctgtttattttgttaattatagagctgtcgtagtaaaaacaaaacattattatgttactcaaaactcttatatgctttttggtgatgtctttaaaatatataatttaaaaggagagataaaaaaaatacatgcattttttccaggagatgaaaataatttatcggatacattaataatgttaaaaacaacaatagttgctgttactacatctaaaggagaagctttgcgtttggctgatgaaattaatatgacatgtaaaatatgtcataagaaaagaaaagcaaccaaattttctatagaaggatttctaagaaaagttagatgtgacacctgtagggttaaaccacgaagtgtaatttgtaaaaatataaataacaataaaaaaacaaacaattcaaaaaataacacaaatagtaataatagtaataataataataataataatataaataataataataataataataataatagtaataatagtaataataaaaatgattcattaaaaccagatgaatgtaatatacagagtaatcaaaataaacatcttacaaaaaataaaattattaaatatgaaaatatagatgacgaaaatataaaaaatgaaaaatttaaaaatatgaatatcaaaaaagttaatacaaaaaattgtaatatcacaaatagtaaggcaaaaaaaactgataataatatacaaaatgatatgaataataatcatcctaacaacaattttgttaataataacattcttgacaataatcatattaataataacattcttgacaataatcatattaataataacattcttgacaataatcatattaataataatcatcttaataataatcatcttaataataatcatcttaataataatcatcttaataataatcatcttaataataaccattttaataataatcatcttaataataatcaacttaacaaccatcttcttgacaatagggataaaatacaatctcttcattacaaacatgcaaattctaactcagtccttaaatataataatagtaataataattactcagctagccaaaatcaaatttttttttcagaggaagaaaatagaactcattttctagagaatgatcccaataagagggatgaaataatttcgatgataattgaacttataaaatatatctcatacatgcaaaaggcattaataaaagcatctgaaggaaattttttgattgaaaatccagacatcaatgtagagaatgtatcaaaattggttcattcagctcaacaattatgtaacaaattacttaaccaaaataatatagacggagaatatgggaataataatttgtgtgcacctacttcgttgcttcttcacaataatgaaaagggaaatacaataaaaaatgatgttaatataaataatataagtaataataataataataataattataattataataataataattataataataataatattaattataataactttagtaataataatcaactaaaaattaaatacacaaaaatgtatcaagcgaaagttaatcacataaccagaacaagcaatttaaataataataataataataataataataatacaaataatacggatatgttttataatatgaacaatccaaatgatatgagcactacaaatactcacatgactaacctttcttatattaatagaaatgaccaaaataaaaataataatatgaataatgatacggatagcaatatatatataaataataaaagcaatgataataatattattaataatataaataatatgaacataatgaataatataaacaatataaataatataaaaaatataaataatataaataatataaaaaatataaataatataaataatattaatagtattaatagtattaataatatgaatcatatgaatcatattaatggtaacaataatattaatagtaacaataatattaatagtaacaataatattaatagtaacaataatattaatagtaacaataatattaatagtaacaataatattaatagtaacaataatatacttaataatacaaatgaaagaaataaatataatattgagaacgaatattcaaaaaatattcaaaatattcaaaatatacaaaatgtacaaaataatcaatcttatgtaataccatattattataataatcgtcataatattaactcttcgacatctttggatacttcaacaaatgatcaaaaaaataaatcatatataaaggataataaagaatatatgtatgaaaataataagaaggatgaacacgaagataaaaaccaatatcatgaccaccaaggcaaacaacaacaacaacaacaacaacaacaacaacaacaacaacaacaacaacaacaacaacatcataataataataatataagtcatacaaaaaataattattacaataataaacagcagttcttattttctactttatcaaaacaatataatactaatgtcaatatttcatttgctaatataaacaattctttatcaaatatttgtaagaatgtctctaaaaattataatctcaaagatacagttttaaatatggaaaagtaa

SEQ ID NO: 36 - protein, 427 aa, Plasmodium falciparum
MHKIDEKINMNNVKGTRIIHVKNTYISPYILHNINNNEGPLKNIDKILKEEKDNINLKINNVNNKETNNDKHTLKYSTLCNDLNIMHTQNKEEEIELNTISTVIHMNSDEDSDKDNDTIINENNDLLIDNIKKYHAVKDNHNNIIYNDKYNTINNNSVINDVCNSIHFNNNSYITNFNLNHNFSVCNRTLNNTCTSQVNLRNNMNSNKKTNDNNKNLNNEINKKINNDNIINEFDNINNKKNNYIDCSVYKCEDEIPLGTILNIQDLDIHNRNNMNNCNNNINNKSKILTTVMLRNIPNKYTQNMLMDVMNEHFKGLYDFFYLPIDFRNKCNVGYAFINFIHPYYAELFIKFFNNYKLNAFKSNKVCSVTWGRVQGLKANIEHYRNSAIMTIPIPQYKPILFQNGITVSWPESDGPLPSIKLRSQKF